SEQUENCE LISTING (24 sequences)

SEQ ID NO: 1 - protein, 22 aa, Artificial Sequence
  Synthetic Construct
  MISC_FEATURE (1)..(5): Xaa can be any amino acid
  MISC_FEATURE (8)..(8): Xaa can be any amino acid
  MISC_FEATURE (14)..(14): Xaa can be any amino acid
  MISC_FEATURE (16)..(16): Xaa can be any amino acid
XXXXXGVXAKAGVXNXFKSESY

SEQ ID NO: 2 - protein, 23 aa, Artificial Sequence
  Synthetic Construct
  MISC_FEATURE (1)..(6): Xaa can be any amino acid and can be either present or absent
  MISC_FEATURE (9)..(9): Xaa can be any amino acid
  MISC_FEATURE (15)..(15): Xaa can be any amino acid
  MISC_FEATURE (17)..(17): Xaa can be any amino acid
XXXXXXGVXAKAGVXNXFKSESY

SEQ ID NO: 3 - protein, 24 aa, Artificial Sequence
  Synthetic Construct
  MISC_FEATURE (3)..(3): Xaa can be any amino acid
  MISC_FEATURE (7)..(7): Xaa can be any amino acid
  MISC_FEATURE (13)..(13): Xaa can be any amino acid
  MISC_FEATURE (22)..(23): Xaa can be either Gln, Pro, Tyr, Ile or Leu
YKXLRRXAPRWDXPLRDPALRXXL

SEQ ID NO: 4 - protein, 23 aa, Artificial Sequence
  Synthetic Construct
  MISC_FEATURE (3)..(3): Xaa can be any amino acid
  MISC_FEATURE (7)..(12): Xaa can be any amino acid and can be either present or absent
  MISC_FEATURE (21)..(22): Xaa can be either Gln, Pro, Tyr, Ile or Leu
YKXLRRXXXXXXPLRDPALRXXL

SEQ ID NO: 5 - protein, 22 aa, Artificial Sequence
  Synthetic Construct
IKLSGGVQAKAGVINMDKSESM

SEQ ID NO: 6 - protein, 22 aa, Artificial Sequence
  Synthetic Construct
IKLSGGVQAKAGVINMFKSESY

SEQ ID NO: 7 - protein, 23 aa, Artificial Sequence
  Synthetic Construct
IKLSGGVQAKAGVINMFKSESYK

SEQ ID NO: 8 - protein, 17 aa, Artificial Sequence
  Synthetic Construct
GVQAKAGVINMFKSESY

SEQ ID NO: 9 - protein, 17 aa, Artificial Sequence
  Synthetic Construct
GVRAKAGVRNMFKSESY

SEQ ID NO: 10 - protein, 17 aa, Artificial Sequence
  Synthetic Construct
  MISC_FEATURE (11)..(11): Xaa is Nle
GVRAKAGVRNXFKSESY

SEQ ID NO: 11 - protein, 24 aa, Artificial Sequence
  Synthetic Construct
YKSLRRKAPRWDAPLRDPALRQLL

SEQ ID NO: 12 - protein, 24 aa, Artificial Sequence
  Synthetic Construct
YKSLRRKAPRWDAYLRDPALRQLL

SEQ ID NO: 13 - protein, 24 aa, Artificial Sequence
  Synthetic Construct
YKSLRRKAPRWDAYLRDPALRPLL

SEQ ID NO: 14 - protein, 17 aa, Artificial Sequence
  Synthetic Construct
  MOD_RES (1)..(1): ACETYLATION
GVRAKAGVRNMFKSESY

SEQ ID NO: 15 - protein, 17 aa, Artificial Sequence
  Synthetic Construct
  MOD_RES (1)..(1): ACETYLATION
  MISC_FEATURE (11)..(11): Xaa is Nle
GVRAKAGVRNXFKSESY

SEQ ID NO: 16 - protein, 24 aa, Artificial Sequence
  Synthetic Construct
  MOD_RES (1)..(1): ACETYLATION
YKSLRRKAPRWDAPLRDPALRQLL

SEQ ID NO: 17 - protein, 24 aa, Artificial Sequence
  Synthetic Construct
  MOD_RES (1)..(1): ACETYLATION
YKSLRRKAPRWDAYLRDPALRQLL

SEQ ID NO: 18 - protein, 24 aa, Artificial Sequence
  Synthetic Construct
  MOD_RES (1)..(1): ACETYLATION
YKSLRRKAPRWDAYLRDPALRPLL

SEQ ID NO: 19 - protein, 23 aa, Homo sapiens
KCLRREAPRWDAPLRDPALRQLL

SEQ ID NO: 20 - protein, 23 aa, Rattus norvegicus
KCLRKKTPRWDILLRDPAPRPLL

SEQ ID NO: 21 - protein, 23 aa, Mus musculus
KCLRKKIPRWDMFLRDPVPRPLL

SEQ ID NO: 22 - protein, 13 aa, Homo sapiens
  MISC_FEATURE (1)..(1): Xaa can be any amino acid
XLYENKPRRPYIL

SEQ ID NO: 23 - protein, 18 aa, Artificial Sequence
  Synthetic Construct
  MISC_FEATURE (11)..(11): Xaa is Nle
GVRAKAGVRNXFKSESYC

SEQ ID NO: 24 - protein, 18 aa, Artificial Sequence
  Synthetic Construct
  MOD_RES (1)..(1): ACETYLATION
  MISC_FEATURE (11)..(11): Xaa is Nle
GVRAKAGVRNXFKSESYC

(19) United States
(12) Patent Application Publication
(10) Pub. No.: US 2023/0036041 A1
(43) Pub. Date: Feb. 2, 2023

(54) SORTILIN BINDING CONJUGATE COMPOUNDS, COMPOSITIONS AND USES THEREOF FOR TREATING CANCER

(71) Applicant: THERATECHNOLOGIES INC., Montréal (CA)

(72) Inventors: Christian MARSOLAIS, Mont Royal (CA); Michel DEMEULE, Beaconsfield (CA); Jean-Christophe CURRIE, Repentigny (CA); Alain LAROCQUE, Saint-Laurent (CA)

(73) Assignee: THERATECHNOLOGIES INC., Montréal, QC (CA)

(21) Appl. No.: 17/781,653

(22) PCT Filed: Dec. 7, 2020
(86) PCT No.: PCT/CA2020/051686

§ 371 Date: Jun. 1, 2022

Related U.S. Application Data
(60) Provisional application No. 62/945,111, filed on Dec. 6, 2019.

(51) Int. Cl. A61K 47/64; A61K 31/337; A61K 47/26; A61K 9/00; A61K 9/08; A61P 35/00; C07K 14/705; C07K 14/78

(52) U.S. Cl. CPC A61K 47/64; A61K 31/337; A61K 47/26; A61K 9/0019; A61K 9/08; A61P 35/00; C07K 14/70571; C07K 14/78; C07K 2319/33

(57) ABSTRACT

The present disclosure relates to compositions comprising a solubilizing agent and a peptide compound and/or a conjugate compound, processes, methods and uses thereof for treatment of cancer or aggressive cancer. For example, the conjugate compounds can comprise the formula of A-(B)n, wherein A is a peptide compound; and B is at least one therapeutic agent, and the peptide compounds can comprise compounds of formula X₁X₂X₃X₄X₅GVX₆AKAGVX₇NX₈FKSESY (I) (SEQ ID NO: 1) (X₉)ₙGVX₁₀AKAGVX₁₁NX₁₂FKSESY (II) (SEQ ID NO: 2) YKX₁₃LRRX₁₄APRWDX₁₅PLRDPALRX₁₆X₁₇L (III) (SEQ ID NO: 3) YKX₁₈LRR(X₁₉)ₙPLRDPALRX₂₀X₂₁L (IV) (SEQ ID NO: 4) IKLSGGVQAKAGVINMDKSESM (V) (SEQ ID NO: 5) IKLSGGVQAKAGVINMFKSESY (VI) (SEQ ID NO: 6) IKLSGGVQAKAGVINMFKSESYK (VII) (SEQ ID NO: 7) GVQAKAGVINMFKSES Y (VIII) (SEQ ID NO: 8) GVRAKAGVRNMFKSESY (IX) (SEQ ID NO: 9) GVRAKAGVRN(Nle)FKSESY (X) (SEQ ID NO: 10) YKSLRRKAPRWDAPLRDPALRQLL (XI) (SEQ ID NO: 11) YKSLRRKAPRWDAYLRDPALRQLL (XII) (SEQ ID NO: 12) YKSLRRKAPRWDAYLRDPALRPLL (XIII) (SEQ ID NO: 13) wherein X₁ to X₂₁ and n can have various different values and wherein at least one protecting group and/or at least one labelling agent is optionally connected to said peptide compound at an N- and/or C-terminal end.

### CROSS REFERENCE TO RELATED APPLICATIONS

The present application claims the benefit of priority from U.S. provisional application No. 62/945,111 filed on Dec. 6, 2019. This document is hereby incorporated by reference in its entirety.

### FIELD OF THE DISCLOSURE

The present disclosure relates to peptide compounds, peptide conjugates, peptidic compositions and related methods and uses thereof.

### BACKGROUND OF THE DISCLOSURE

According to a recent World Health Organization report, 8.2 million patients died from cancer in 2012 (1). Cancer is therefore a continuously growing health problem in both developing and developed countries. It has also been estimated that the number of annual cancer cases will increase within the next two decades (1). The common general treatments for cancer are surgery, endocrine therapy, chemotherapy, and radiotherapy (2). A recent hope has however been put in the generation of “targeted therapeutics” that home-in on specific molecular defects in cancer cells, promising more effective and less toxic therapies than imprecise chemotherapeutic agents (3).

Currently, when anticancer drugs are administered through classical formulations, it is estimated that ˜95% of the therapeutic agent is taken up by cells within healthy tissues, whereas only ˜2-5% effectively reaches tumors (4). The challenge in any future successful personalized therapeutic approach is therefore to increase selectivity of targeting therapy in part through active transport of anticancer drugs into cancer cell compartments (5-6).

Given its role in ligand internalization and cellular trafficking, Sortilin can be considered as one of the cells' own shuttle system (11). Recent studies demonstrated that Sortilin has a dual role both in endocytosis and in receptor trafficking allowing the sorting of ligands from the cell surface to specific subcellular compartments and the trafficking of pro-neurotrophins such as the neuropeptide neurotensin (NT), proNGF and proBDNF (8, 11-16). Sortilin expression is elevated in several human cancers including breast, prostate, colon, pancreas, skin, and pituitary (17-20). Sortilin has also been reported to be overexpressed in ovarian cancers as compared to healthy ovarian tissue (21, 22).

### SUMMARY OF THE DISCLOSURE

Accordingly, a first aspect is a composition comprising a solubilizing agent and a peptide compound or a pharmaceutically acceptable salt, solvate or prodrug thereof, said peptide compound having at least 60% sequence identity to a compound chosen from compounds of formula (I), formula (II), formula (III), formula (IV), formula (V), formula (VI), formula (VII), formula (VIII), formula (IX), formula (X), formula (XI) and formula (XII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid;
and wherein at least one protecting group and/or at least one labelling agent is optionally connected to said peptide at an N- and/or C-terminal end,
optionally the peptide compound is cyclic.

Another aspect is a composition comprising a solubilizing agent and a peptide compound or a pharmaceutically acceptable salt, solvate or prodrug thereof, said peptide compound having at least 80% sequence identity to a compound chosen from compounds of formula (I), formula (II), formula (III), formula (IV), formula (V), formula (VI), formula (VII), formula (VIII), formula (IX), formula (X), formula (XI) and formula (XII):

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | (XIII) |
| (SEQ ID NO: 13) | |
| | YKSLRRKAPRWDAYLRDPALRPLL |

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L; n is 0, 1, 2, 3, 4 or 5;
when X₉ is present more than once, each of said X₉ is independently chosen from any amino acid;
when X₁₉ is present more than once, each of said X₉ is independently chosen from any amino acid;
and wherein at least one protecting group and/or at least one labelling agent is optionally connected to said peptide at an N- and/or C-terminal end,
optionally the peptide compound is cyclic.

In an aspect, there is provided a composition comprising a solubilizing agent and a peptide compound or derivative thereof that targets Sortilin receptor.

In an aspect, there is provided a composition comprising a solubilizing agent and a peptide compound or derivative thereof for use in targeting Sortilin receptor.

In a further aspect disclosed herein is a composition comprising a solubilizing agent and a conjugate compound or a pharmaceutically acceptable salt, solvate or prodrug thereof, said conjugate compound having the formula of A-(B)ₙ,

wherein
n is 1, 2, 3 or 4;
A is a peptide compound as defined in the present disclosure, wherein said peptide is optionally protected by a protecting group; and
B is at least one therapeutic agent, wherein B is connected to A.

In a further aspect disclosed herein is a composition comprising a solubilizing agent and a conjugate compound or a pharmaceutically acceptable salt, solvate or prodrug thereof, said conjugate compound having the formula of A-(B)ₙ,

wherein
n is 1, 2, 3 or 4;
A is a peptide compound as defined in the present disclosure, wherein said peptide compound is optionally protected by a protecting group; and
B is at least one therapeutic agent, wherein B is connected to A, optionally at a free amine of said peptide compound, at an N-terminal position of said peptide compound, at a free —SH of said peptide compound, or at a free carboxyl of said peptide compound.

A further aspect disclosed herein is a composition comprising a solubilizing agent and a conjugate compound or a pharmaceutically acceptable salt, solvate or prodrug thereof, said conjugate compound having the formula of A-(B)ₙ,

wherein
n is 1, 2, 3 or 4;
A is a peptide compound as defined in the present disclosure, wherein said peptide is optionally protected by a protecting group; and
B is at least one therapeutic agent, wherein B is connected to A at a free amine of a lysine residue of said peptide compound, optionally via a linker, or at an N-terminal position of said peptide compound, optionally via a linker.

Another aspect disclosed herein is a composition comprising a solubilizing agent and a conjugate compound or a pharmaceutically acceptable salt, solvate or prodrug thereof, said conjugate compound represented by formula (XXIII):

Acetyl-GVRAK(docetaxel)AGVRN(Nle)FK(docetaxel)SESY Formula (XXIII)

that comprises the peptide compound having SEQ ID NO: 15 wherein each lysine residue has a docetaxel molecule connected thereto.

Another aspect disclosed herein is a composition comprising a solubilizing agent and a conjugate compound or a pharmaceutically acceptable salt, solvate or prodrug thereof, said conjugate compound represented by formula (XXVIII):

Acetyl-GVRAK(doxorubicin)AGVRN(Nle)FK(doxorubicin)SESY Formula (XXVIII)

that comprises the peptide compound having SEQ ID NO: 15 wherein each lysine residue has a doxorubicin molecule connected thereto.

Another aspect disclosed herein is a composition comprising a solubilizing agent and a conjugate compound or a pharmaceutically acceptable salt, solvate or prodrug thereof, said conjugate compound represented by formula (LII):

Acetyl-GVRAKAGVRN(Nle)FKSESYC(aldoxorubicin) Formula (LII)

that comprises the peptide compound having SEQ ID NO: 24 wherein cysteine residue has an aldoxorubicin molecule connected thereto, or
that comprises the peptide compound having SEQ ID NO: 15 wherein a cysteine residue is added to C-terminal of said peptide compound, and wherein the cysteine residue has an aldoxorubicin molecule connected thereto.

Another aspect disclosed herein is a composition comprising a solubilizing agent and a conjugate compound or a pharmaceutically acceptable salt, solvate or prodrug thereof, said conjugate compound chosen from compounds of formula (XVI) and formula (XVII):

Acetyl-GVRAK(curcumin)AGVRN(Nle)FK(curcumin)SESY Formula (XVI)

that comprises the peptide compound having SEQ ID NO: 15 wherein each lysine residue has a curcumin molecule connected thereto; and

Acetyl-YK(curcumin)SLRRK(curcumin)APRWDAPLRDPALRQLL Formula (XVII)

that comprises the peptide compound having SEQ ID NO: 16 wherein each lysine residue has a curcumin molecule connected thereto.

In another aspect, there is provided a method for increasing half life and/or stability of i) a peptide compound or a pharmaceutically acceptable salt, solvate or prodrug thereof, said peptide having at least 60% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII) or ii) a conjugate compound having the formula of A-(B)ₙ,

wherein
n is 1, 2, 3 or 4;
A is said peptide compound; and
B is at least one therapeutic agent, wherein B is connected to A at a free amine of a lysine residue of said peptide compound, optionally via a linker, or at an N-terminal position of said peptide compound, optionally via a linker,
comprising mixing the peptide compound or the conjugate compound with a solubilizing agent so as to increase the half life by at least 2 fold, at least 2.5 fold, at least 3 fold, at least 3.5 fold, at least 4 fold, at least 4.5 fold, at least 5 fold, at least 5.5 fold, or at least 6 fold.

In another aspect, there is provided a method for increasing half life and/or stability of a peptide or a pharmaceutically acceptable salt, solvate or prodrug thereof, said peptide having at least 60% sequence identity to a compound chosen from compounds of formula (I), formula (II), formula (III), formula (IV), formula (V), formula (VI), formula (VI), formula (VII), formula (IX), formula (X), formula (XI), formula (XII) and formula (XIII), said method comprising conjugating the peptide compound with at least one molecule.

In another aspect, there is provided a method of treating cancer or aggressive cancer, comprising administering to a subject in need thereof a therapeutically effective amount of at least one composition as defined herein.

In an aspect, there is provided a use of a composition as defined herein for treating a cancer.

In an aspect, there is provided a use of a composition as defined herein for targeting Sortilin receptor.

In an aspect, there is provided a use of a composition as defined herein for treatment of cancer or aggressive cancer.

In an aspect, there is provided a use of a composition as defined herein for treatment of cancer or aggressive cancer in cancerous tissues or cells expressing Sortilin.

In an aspect, there is provided a use of a composition as defined herein in the manufacture of a medicament for treating a cancer.

In an aspect, there is provided a use of a composition as defined herein in the manufacture of a medicament for targeting Sortilin receptor.

In an aspect, there is provided a use of a composition as defined herein in the manufacture of a medicament for treatment of cancer or aggressive cancer.

In an aspect, there is provided a use of a composition as defined herein in the manufacture of a medicament for treatment of cancer or aggressive cancer in cancerous tissues or cells expressing Sortilin.

In another aspect, there is provided a use of a composition as defined herein in the manufacture of a medicament for targeting Sortilin receptor.

In a further aspect, there is provided a liposome, graphene, nanotube or nanoparticle comprising a composition as defined herein.

In a further aspect, there is provided a liposome, graphene, nanotube or nanoparticle comprising a composition as defined herein for use in targeting Sortilin receptor.

### BRIEF DESCRIPTION OF THE FIGURES

Further features and advantages of the disclosure will become more readily apparent from the following description of specific embodiments as illustrated by way of examples in the appended schemes and figures wherein:

FIG. 1 is a tissue immunohistochemistry microarray showing high expression of sortilin in human breast cancers (infiltrating ductal carcinoma and lymph node metastatic carcinoma).

FIG. 2 is a bar graph showing level of sortilin expression in infiltrating ductal carcinoma, lymph node metastatic carcinoma, TNBC vs normal tissue.

FIG. 3 is a graph showing survival rate in TNBC patients with high vs low sortilin expression.

FIG. 4 is a graph showing survival rate in TNBC with lymph node metastases with high vs low sortilin expression.

FIG. 5 is a western blot image showing that Sortilin is highly expressed in different human TNBC cancer cell lines.

FIG. 6 is a bar graph showing inhibition of uptake of the peptide TH19P01 upon sortilin siRNA.

FIG. 7 is a bar graph showing apoptosis of MDA-MB-231 cells in docetaxel vs TH1902 treated cells, as a function of concentration and time.

FIG. 8 is a bar graph showing reversal of apoptosis of TH1902 treated MDA-MB-231 cells by sortilin ligands TH19P01, neurotensin and progranulin.

FIG. 9 is a series of images showing immunostaining of α-tubulin in MDA-MB-231 cells treated with docetaxel or TH1902 vs control.

FIG. 10 is a graph showing cell migration being inhibited by TH1902 in a sortilin dependent-manner.

FIG. 11 is a bar graph showing neutrophil count as a function of number of treatments with docetaxel or TH1902.

FIG. 12 is a graph showing the concentration of TH1902 and released docetaxel in plasma of mice injected i.v. with TH1902 as a function of time.

FIG. 13 is a graph showing tumor volume in mice treated with vehicle, high docetaxel dose or TH1902 (at equivalent docetaxel dose) as a function of time.

FIG. 14 is a graph showing tumor volume in mice treated with vehicle, low docetaxel dose or TH1902 (at equivalent docetaxel dose) as a function of time.

FIG. 15 is a graph showing stability of DoceKA (i.e. TH1902 conjugate) when formulated vs when dissolved in DMSO.

FIG. 16 is a graph showing MDA-MB-231 tumor volume in mice treated with vehicle, docetaxel or various TH1902 formulations, as a function of time.

FIG. 17 is a graph showing tumor volume in vehicle, docetaxtel or TH1902 (Formulation 2) treated mice as a function of time.

FIG. 18 is a graph showing weight of mice treated with vehicle, docetaxtel or TH1902 (Formulation 2), as a function of time.

FIG. 19 is a graph showing MDA-MB231 tumor volumes in mice treated with vehicle or various TH1902 formulations, as a function of time.

FIG. 20 is a bar graph showing tumor progression in mice treated with vehicle or various TH1902 formulations (at 17.5 mg/kg/week).

FIG. 21 is a graph showing heating profile during dissolution of TH1902 API of R&D Stability lab batch (Example 5A).

FIG. 22 is a graph showing heating profile during dissolution of TH1902 API with the internal procedure (Example 5B).

FIG. 23 is a representative UPLC analysis of the Stock Solution of TH1902 at 10 mg/ml following dissolution with the internal procedure (Example 5B).

FIG. 24 shows results in endometrial cancer xenograft model (AN3-CA) of mice treated with vehicle, low and high docetaxel doses or TH1902 at low and high doses: A) is a graph showing tumor volume as a function of time, B) is a bar graph showing tumor progression at study endpoint and C) is a graph showing mouse body weight as a function of time.

FIG. 25 shows results in colorectal cancer xenograft model (HT-29) of mice treated with vehicle, docetaxel or TH1902: A) is a graph showing tumor volume as a function of time at low dose, B) is a graph showing tumor volume as a function of time at high dose, C) is a graph showing mouse body weight as a function of time at low dose, D) is a graph showing mouse body weight as a function of time at high dose, E) is a bar graph showing tumor progression at study endpoint at low dose, F) is a bar graph showing tumor progression at study endpoint at high dose.

FIG. 26 shows results in pancreatic cancer xenograft model (PANC-1) of mice treated with vehicle, low and high docetaxel doses or TH1902 at low and high doses: A) and B) are graphs showing tumor volume as a function of time and C) is a bar graph showing tumor progression at study endpoint.

FIG. 27 shows results in melanoma cancer xenograft model (SK-Mel-28) of mice treated with vehicle, low and high docetaxel doses or TH1902 at low and high doses: A) is a graph showing tumor volume as a function of time, B) is a bar graph showing tumor progression at study endpoint, and C) is a graph showing mouse body weight as a function of time.

FIG. 28 shows results in syngeneic melanoma tumor model (B16F10) of mice treated with vehicle, high docetaxel dose or TH1902 at high dose A) is a graph showing tumor volume as a function of time, B) is a bar graph showing tumor progression at study endpoint, C) is a graph showing mouse body weight as a function of time, and D) is an image showing ex vivo tumors at study endpoint.

FIG. 29 shows dose-response results in syngeneic melanoma tumor model (B16F10) of mice treated with vehicle, docetaxel or TH1902 at three equivalent increasing doses: A) is a graph showing tumor volume as a function of time, B) is a bar graph showing tumor progression at study endpoint, C) is a graph showing mouse body weight as a function of time.

### DETAILED DESCRIPTION OF THE DISCLOSURE

The term “peptide compounds” as used herein refers, for example, to peptides derived from bacterial proteins or from ligands of receptors that target receptors expressed on cancer cells including multidrug resistant cancer cells. For example, the peptide compounds can be derived from bacterial proteins involved in cell penetration or from sortilin ligands, for example progranulin and neurotensin. For example, the peptide compounds can be cyclic. In certain embodiments, peptide compounds are connected (for example via a covalent bond, an atom or a linker) to at least one therapeutic agent (such as an anticancer agent or a phytochemical), thereby forming a conjugate compound that can be used, for example, for treatment of a cancer or aggressive cancer. In certain other embodiments, peptide compounds can be used at the surface of liposomes. For example, the peptide compounds can be used for coating liposomes, graphene, nanotubes or nanoparticles that can be loaded with at least one therapeutic agent (such as an anticancer agent or phytochemical, or genes or siRNA).

The term “KBP Family 1 peptide compounds” refers to peptide compounds derived from bacterial cell penetrant proteins. For example, KBP Family 1 peptide compounds can be derived from a protein having an amino acid sequence of IKLSGGVQAKAGVINMDKSESM (SEQ ID NO: 5). Non limiting examples of KBP Family 1 peptide compounds are shown below:

Amino acid sequences

| KBP- | IKLSGGVQAKAGVINMDKSESM - Formula (V) |
|---|---|
| 101 | (represented by SEQ ID NO: 5) |
| KBP- | Succinyl-IKLSGGVQAKAGVINMFKSESY - Formula |
| 102 | (XXXVI) (comprises SEQ ID NO: 6 wherein a |
| | succinyl group is attached at the N-terminal |
| | end) |
| KBP- | IKLSGGVQAKAGVINMFKSESYK(Biotin) - Formula |
| 103 | (XXXVII) (comprises SEQ ID NO: 7 wherein a |
| | biotin molecule is connected thereto at the |
| | C-terminal end) |
| KBP- | GVQAKAGVINMFKSESY - Formula (VIII) |
| 104 | (represented by SEQ ID NO: 8) |
| KBP- | Acetyl-GVRAKAGVRNMFKSESY - Formula (XXXVIII) |
| 105 | (represented by SEQ ID NO: 14) |
| KBP- | Acetyl-GVRAKAGVRN(Nle)FKSESY - Formula |
| 106 | (XXXIX) (represented by SEQ ID NO: 15) |

As used herein, the peptide compound KBP-101 is represented by the amino acid sequence of IKLSGGVQAKAGVINMDKSESM (SEQ ID NO: 5).

As used herein, the peptide compound KBP-102 is represented by the amino acid sequence of Succinyl-IKLSGGVQAKAGVINMFKSESY that comprises the peptide sequence of SEQ ID NO: 6 wherein a succinyl group is attached thereto at the N-terminal end.

As used herein, the peptide compound KBP-103 is represented by the amino acid sequence of IKLSGGVQAKAGVINMFKSESYK(Biotin) that comprises the peptide sequence of SEQ ID NO: 7 wherein a biotin molecule is connected thereto at the C-terminal end.

As used herein, the peptide compound KBP-104 is represented by the amino acid sequence of GVQAKAGVINMFKSESY (SEQ ID NO: 8).

As used herein, the peptide compound KBP-105 is represented by the amino acid sequence of Acetyl-GVRAKAGVRNMFKSESY (SEQ ID NO: 14).

As used herein, the peptide compound KBP-106 is represented by the amino acid sequence of Acetyl-GVRAKAGVRN(Nle)FKSESY (SEQ ID NO: 15).

As used herein, “TH19P01” or “TH19P01 peptide” is synonymous with the peptide compound KBP-106 represented by the sequence of SEQ ID NO: 15.

The term “KBP Family 2 peptide compounds” refers to peptides derived from sortilin ligands, progranulin and neurotensin. For example, peptides can be derived from human, rat or mouse progranulin. For example, KBP Family 2 peptide compounds can be derived from human progranulin, for example having the amino acid sequence KCLRREAPRWDAPLRDPALRQLL (SEQ ID NO: 19), from rat progranulin, for example having the amino acid sequence KCLRKKTPRWDILLRDPAPRPLL (SEQ ID NO: 20), from mouse progranulin, for example having the amino acid sequence KCLRKKIPRWDMFLRDPVPRPLL (SEQ ID NO: 21), or from neurotensin, for example having an amino acid sequence XLYENKPRRPYIL (SEQ ID NO: 22). Non limiting examples of KBP Family 2 peptide compounds are shown below:

Amino acid sequences

| | |
|---|---|
| KBP-201 | Acetyl-YKSLRRKAPRWDAPLRDPALRQLL - |
| | Formula (XXXX) (represented by |
| | SEQ ID NO: 16) |
| KBP-202 | Acetyl-YKSLRRKAPRWDAYLRDPALRQLL - |
| | Formula (XXXXI) (represented by |
| | SEQ ID NO: 17) |
| KBP-203 | Acetyl-YKSLRRKAPRWDAYLRDPALRPLL - |
| | Formula (XXXXII) (represented by |
| | SEQ ID NO: 18) |

As used herein, the peptide compound KBP-201 is represented by the amino acid sequence of Acetyl-YKSLRRKAPRWDAPLRDPALRQLL (SEQ ID NO: 16).

As used herein, the peptide compound KBP-202 is represented by the amino acid sequence of Acetyl-YKSLRRKAPRWDAYLRDPALRQLL (SEQ ID NO: 17).

As used herein, the peptide compound KBP-203 is represented by the amino acid sequence of Acetyl-YKSLRRKAPRWDAYLRDPALRPLL (SEQ ID NO: 18).

The term “Sortilin” or “Sortilin receptor” as used herein refers to a neuronal type-1 membrane glycoprotein, encoded by the SORT1 gene, belonging to the Vacuolar Protein Sorting 10 protein (Vps10) family of receptors. Sortilin (also known as the neurotensin receptor 3, Accession number NP_002950, herein incorporated by reference) is expressed abundantly in the central and peripheral nervous systems and is also expressed in other types of tissues. For example, the expression of sortilin is upregulated in a number of cancers including for example ovarian, breast, colon and prostate cancer. The encoded preproprotein is proteolytically processed by furin to generate the mature receptor with a molecular weight of 100-110 kDa. A truncated and soluble form of Sortilin (95 kDa) has also been described, corresponding to its large luminal domain (i.e. extracellular domain or ectodomain), which has been previously detected in the supernatant medium from sortilin-overexpressing cells (48). Amino acid residues of sortilin referenced herein correspond to positions in the full-length form (i.e. Accession number NP_002950). The extracellular domain of Sortilin is at amino acid residues 78-755 of the full-length form. The peptide compounds and conjugate compounds herein described can have a high binding affinity to sortilin and thus can specifically target cancer cells expressing or overexpressing sortilin.

The term “compound” as used in the present document refers to compounds of formulas (I), (II), (III), (IV), (V), (VI), (VII), (VIII), (IX), (X), (XI), (XII), (XIII), (XIV), (XV), (XVI), (XVII), (XIX), (XXIII), (XXVI), (XXVIII), (LI), (LII) or to pharmaceutically acceptable salts, solvates, hydrates and/or prodrugs of these compounds, isomers of these latter compounds, or racemic mixtures of these latter compounds, and/or to composition(s) made with such compound(s) as previously indicated in the present disclosure. The expression “compound” also refers to mixtures of the various compounds herein disclosed.

Compounds of the present disclosure include prodrugs. In general, such prodrugs will be functional derivatives of these compounds which are readily convertible in vivo into the compound from which it is notionally derived. Prodrugs of the compounds of the present disclosure may be conventional esters formed with available hydroxy, or amino group. For example, an available OH or nitrogen in a compound of the present disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the present disclosure are those in which one or more of the hydroxy groups in the compounds are masked as groups which can be converted to hydroxy groups in vivo. Conventional procedures for the selection and preparation of suitable prodrugs are described, for example, in “Design of Prodrugs” ed. H. Bundgaard, Elsevier, 1985.

Compounds of the present disclosure include radiolabeled forms, for example, compounds labeled by incorporation within the structure ²H, ³H, ¹⁴C, ¹⁵N, or a radioactive halogen such as ¹²⁵I. A radiolabeled compound of the compounds of the present disclosure may be prepared using standard methods known in the art.

The term “analog” as used herein includes parts, extensions, substitutions, variants, modifications or chemical equivalents and derivatives thereof of the amino acid of the present disclosure that perform substantially the same function as the peptide or antigen of the disclosure in substantially the same way. For example, analogs of peptides and antigens of the disclosure include, without limitation, conservative amino acid substitutions. Analogs of the peptides and antigens of the disclosure also include additions and deletions to the peptides and antigens of the disclosure.

A “conservative amino acid substitution”, as used herein, is one in which one amino acid residue is replaced with another amino acid residue without abolishing the peptide's or antigen's desired properties.

The expression “derivative thereof” as used herein when referring to a compound means a derivative of the compound that has a similar reactivity and that could be used as an alternative to the compound in order to obtain the same desired result.

The term “cancer” as used herein means a primary or a secondary cancer and includes a non-metastatic cancer and/or a metastatic cancer. Reference to cancer includes reference to cancer tissues or cells. For example, the cancer is ovarian cancer, brain cancer, breast cancer (e.g. triple negative breast cancer), melanoma, colorectal cancer, glioblastoma, liver cancer, lung cancer, prostate cancer, cervical cancer, head cancer, gastric cancer, kidney cancer, endometrial cancer, testis cancer, urothelial cancer, acute lymphoblastic leukemia, acute myeloid leukemia, chronic lymphocytic leukemia, Hodgkin lymphoma, neuroblastoma, non-Hodgkin lymphoma, soft tissue cancer, bone sarcoma, thyroid cancer, transitional cell bladder cancer, Wilm's tumour, glioma, pancreatic cancer or spleen cancer. The term “cancer” as used herein also comprises any cancer involving expression of Sortilin.

The term “aggressive cancer” as used herein refers to cancer having cancer cells that are rapidly dividing and growing. Aggressive cancer may be invasive or metastatic or is more likely to be invasive or metastatic and to spread to lymph nodes and/or other body organs. Reference to aggressive cancer includes reference to aggressive cancer tissues or cells. Aggressive cancer can be any of cancer type described herein.

The expression “therapeutic agent” as used herein means an agent capable of producing a therapeutic effect by inhibiting, suppressing or decreasing a cancer (e.g., as determined by clinical symptoms or the amount of cancerous cells) in a subject, in a cancerous tissue, or in cells, compared to a control. Examples of therapeutic agents include for example anticancer agents and phytochemicals.

The term “anticancer agent” as used herein means an agent capable of causing toxicity in cancer cells. For example, taxanes, which are derived from the bark of the Pacific yew tree Taxus brevifolia, can be used as anticancer agents. Taxanes include for example docetaxel, paclitaxel and cabazitaxel. Other anticancer agents include for example anthracycline compounds which work by intercalating DNA. For example, anthracyclines include doxorubicin and aldoxorubicin.

The term “docetaxel” or “doce” as used herein means an anticancer agent having the structure:

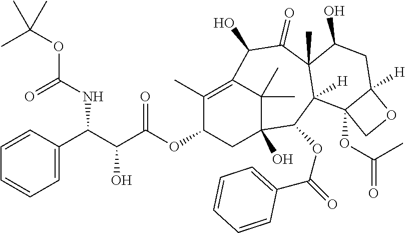

or pharmaceutically acceptable salts, solvates or prodrugs thereof as well as mixtures thereof. For example, docetaxel can be conjugated to a peptide compound of the present disclosure via the oxygen atom attached to the carbon atom at position 2 of its side chain. Docetaxel can be connected to the peptide compound directly or via a linker.

The term “doxorubicin”, “dox” or “doxo” as used herein means an anticancer agent having the structure:

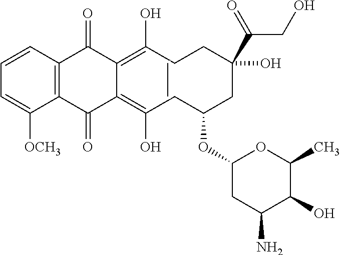

or pharmaceutically acceptable salts, solvates or prodrugs thereof as well as mixtures thereof. For example, doxorubicin can be conjugated to a peptide compound of the present disclosure via the oxygen atom attached to the carbon atom at position 14. Doxorubicin can be connected to the peptide compound directly or via a linker.

The term “cabazitaxel” or “cab” as used herein means an anticancer agent having the structure:

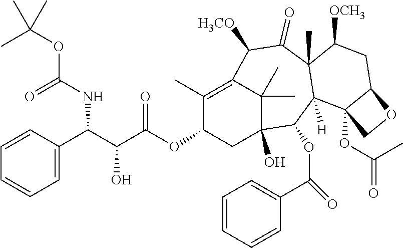 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

or pharmaceutically acceptable salts, solvates or prodrugs thereof as well as mixtures thereof. For example, cabazitaxel can be conjugated to a peptide compound of the present disclosure via the oxygen atom attached to the carbon atom at position 2 of its side chain. Cabazitaxel can be connected to the peptide compound directly or via a linker.

The term “aldoxorubicin” or “aldo” as used herein means an anticancer agent having the structure:

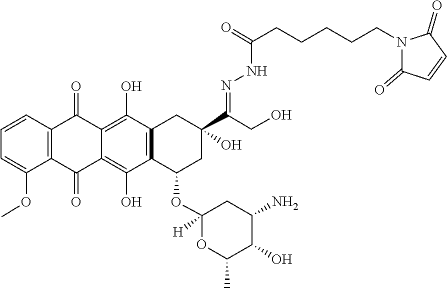 [SMILES: COc1cccc2c1C(=O)c1c(O)c3c(c(O)c1C2=O)C[C@@](O)(/C(CO)=N/NC(=O)CCCCCN1C(=O)C=CC1=O)C[C@@H]3O[C@H]1C[C@H](N)[C@H](O)[C@H](C)O1]

or pharmaceutically acceptable salts, solvates or prodrugs thereof as well as mixtures thereof. For example, aldoxorubicin can be conjugated to a peptide compound of the present disclosure via the (6-maleimidocaproyl) hydrazone attached to the carbon in position 13 of its side chain. Aldoxorubicin can be connected to the peptide compound directly or via its linker.

The term “phytochemical” as used herein means chemical compounds that occur naturally in plants and that can be used for treating a cancer. Examples of phytochemicals include for example Curcumin. Curcumin (diferuloylmethane) is a yellow pigment present in the spice turmeric (Curcuma longa) that has been associated with anti-inflammatory. Other phytochemicals with anti-inflammatory properties include for example omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

The term “curcumin” or “cur” as used herein means a phytochemical having the structure:

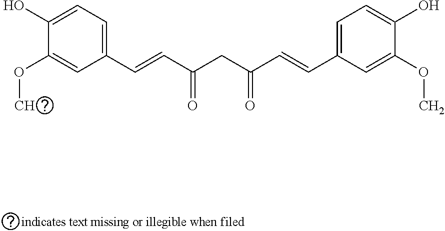 [SMILES: COc1cc(/C=C/C(=O)CC(=O)/C=C/c2ccc(O)c(OC)c2)ccc1O]

or pharmaceutically acceptable salts, solvates or prodrugs thereof as well as mixtures thereof. For example, curcumin can be conjugated to a peptide compound of the present disclosure via an oxygen atom of its phenol groups. Curcumin can be connected to the peptide compound directly or via a linker.

The expression “conjugate compounds”, “peptide-drug conjugates”, or “peptide conjugates” as used herein refers to compounds comprising a peptide compound herein disclosed connected to at least one therapeutic agent, optionally via a linker. Conjugate compound can comprise, for example, 1, 2, 3 or 4 molecules of a therapeutic agent connected thereto. These 1-4 molecules of therapeutic agent can be the same or different i.e. up to four different therapeutic agents could be connected to the peptide compounds. The therapeutic agent(s) are connected to the peptide compound via at least one covalent bond, at least one atom or at least one linker. Conjugate compounds can be used for treating a cancer. Examples of conjugate compounds include, without limitation, the conjugate compounds shown below:

| | |
|---|---|
| KBC-106 | Acetyl-GVRAK(curcumin)AGVRN(Nle) |
| (2:1) | FK(curcumin) SESY - Formula (XVI) |
| | that comprises the peptide compound |
| | having SEQ ID NO: 15 wherein each |
| | lysine residue has a curcumin |
| | molecule connected thereto |
| KBC-201 | Acetyl-YK(curcumin)SLRRK(curcumin) |
| (2:1) | APRWDAPLRDPALRQLL - Formula (XVII) |
| | that comprises the peptide compound |
| | having SEQ ID NO: 16 wherein each |
| | lysine residue has a curcumin |
| | molecule connected thereto |

The term “conjugating” as used herein, refers, for example, to the preparation of a conjugate as defined above. Such an action comprises connecting a peptide compound together with at least one therapeutic agent, optionally via a linker.

For example, the following are general chemical formulas of some peptide-conjugate compounds herein disclosed.

Curcumin-peptide conjugate compound:

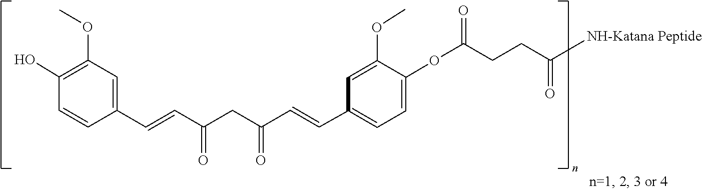

For example, the following are the chemical structures of some conjugate compounds herein disclosed.

### Docetaxel-Peptide Conjugate (DoceKA) (TH1902):

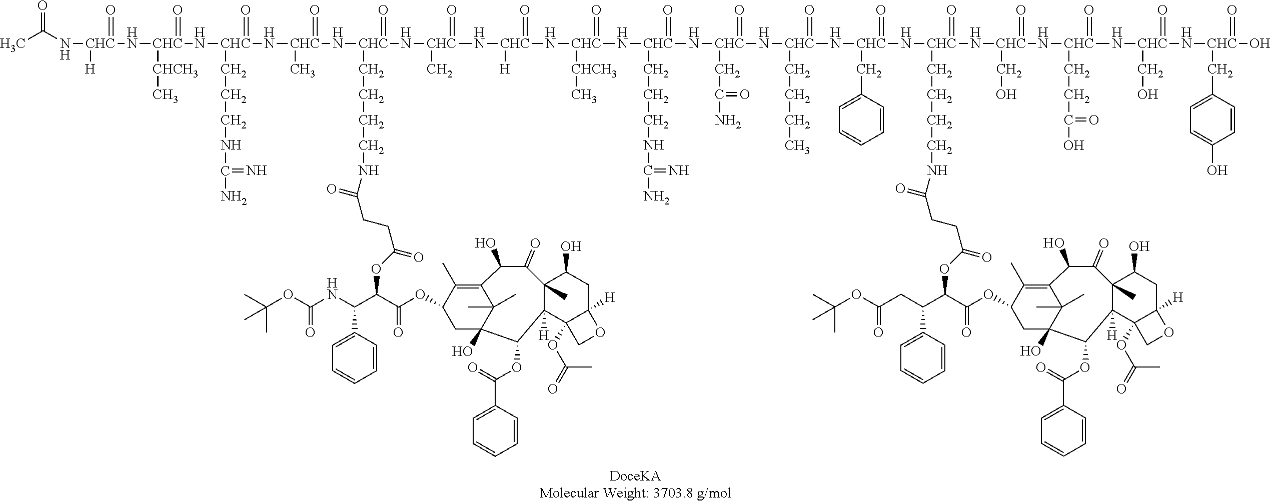

### Doxorubicin-Peptide Conjugate (DoxKA):

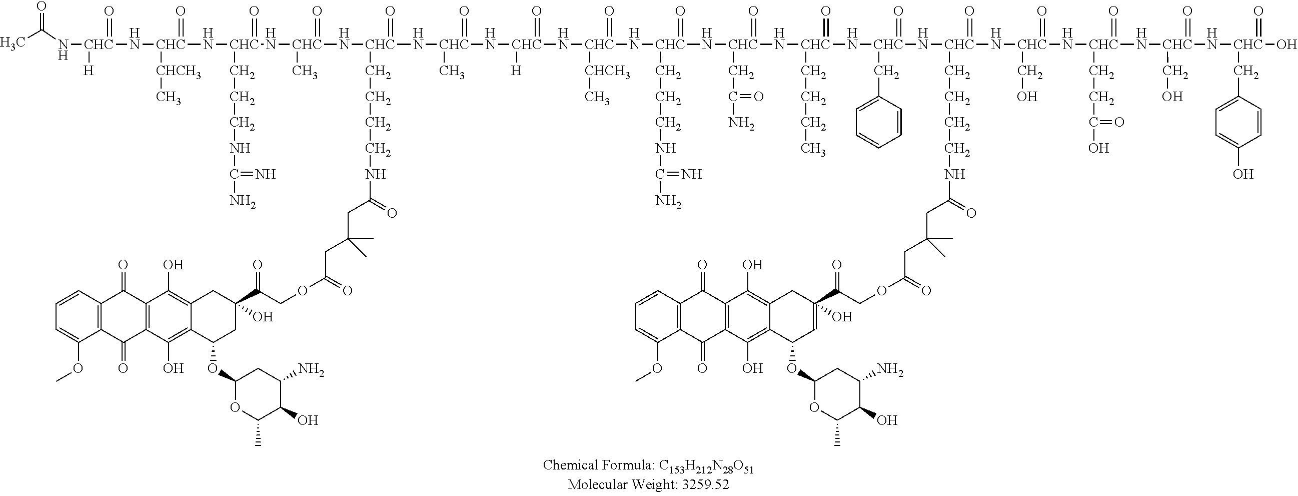

### IBC-106:

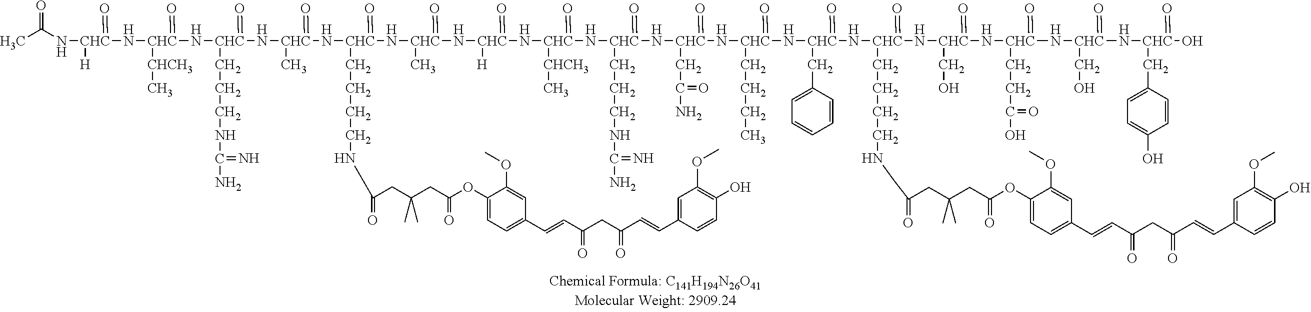

### Curcumin-Peptide Conjugates:

### KBC-201:

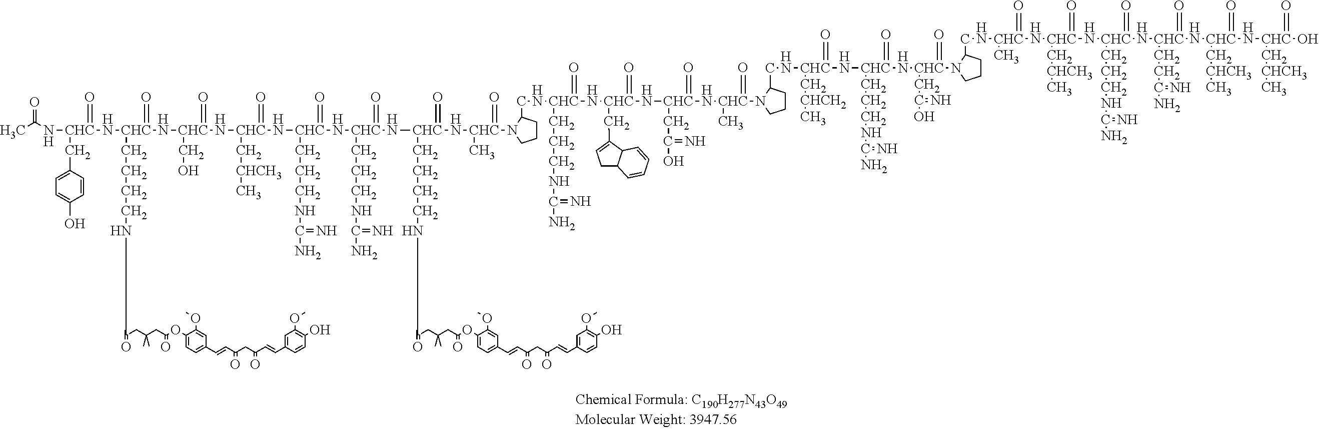

### KBP-106-Cys-Aidorubicin:

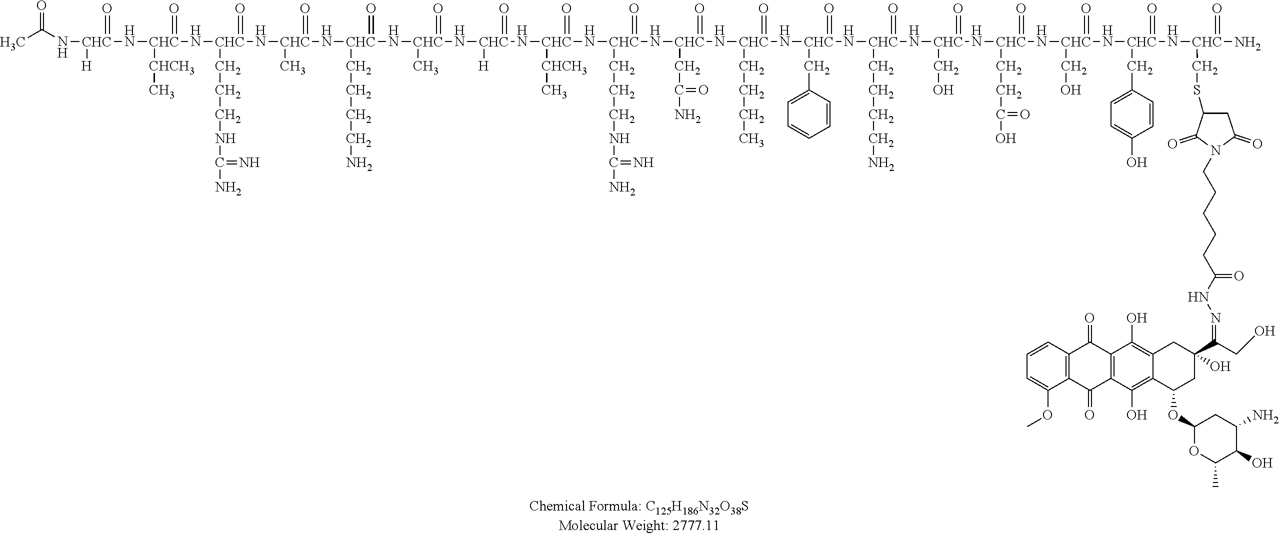

The term “linker” as used herein means a chemical structure connecting a peptide compound herein disclosed to at least one therapeutic agent. The linker can be connected to the peptide compound at different functional groups on the peptide compound. For example, the linker can be connected to the peptide compound at the primary amines (amines (—NH2): this group exists at the N-terminus of each polypeptide chain (called the alpha-amine) and in the side chain of lysine (Lys, K) residues (called the epsilon-amine). For example, the linker can be connected to the peptide compound at the carboxyls (—COOH): this group exists at the C-terminus of each polypeptide chain and in the side chains of aspartic acid (Asp, D) and glutamic acid (Glu, E). For example, the linker can be connected to the peptide compound at the Sulfhydryls (—SH): This group exists in the side chain of cysteine (Cys, C). Often, as part of a protein's secondary or tertiary structure, cysteines are joined together between their side chains via disulfide bonds (—S—S—). These must be reduced to sulfhydryls to make them available for crosslinking by most types of reactive groups. For example, the linker can be connected to the peptide compound at the Carbonyls (—CHO): Ketone or aldehyde groups can be created in glycoproteins by oxidizing the polysaccharide post-translational modifications (glycosylation) with sodium meta-periodate. For example, the linker can be a cleavable linker. For example, the linker can be a non-cleavable linker.

The following table summarizes the reactivity class and the chemical group of some of the principal linkers for standard chemical conjugation:

| Reactivity class | Chemical group |
|---|---|
| Carboxyl-to-amine reactive groups | Carbodiimide (e.g., EDC) |
| Amine-reactive groups | NHS ester |
| | Imidoester |
| | Pentafluorophenyl ester |
| | Hydroxymethyl phosphine |
| Sulfhydryl-reactive groups | Maleimide |
| | Haloacetyl (Bromo- or Iodo-) |
| | Pyridyldisulfide |
| | Thiosulfonate |
| | Vinylsulfone |
| Aldehyde-reactive groups | Hydrazide |
| i.e., oxidized sugars (carbonyls) | Alkoxyamine |
| Photoreactive groups | Diazirine |
| | Aryl Azide |

For example, homobifunctional and heterobifunctional crosslinkers can be used. For example, Disuccinimidyl suberate (DSS) is a homobifunctional crosslinker that has identical amine-reactive NHS-ester groups at either end of a short spacer arm. For example, Sulfosuccinimidyl 4-(N-maleimidomethyl)cyclohexane-1-carboxylate (Sulfo-SMCC) is a heterobifunctional crosslinker that has an amine-reactive sulfo-NHS-ester group at one end and a sulfhydryl reactive maleimide group at the opposite end of a cyclohexane spacer arm. This allows for sequential, two-step conjugation procedures. Among the commercially available homobifunctional cross-linkers are: BSOCOES (Bis(2-[Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); and BASED (Bis(β-[4-azidosalicylamido]-ethyl)disulfide iodinatable).

The peptide compounds may be conjugated through a variety of linkers, e.g., sulfhydryl groups, amino groups (amines), or any appropriate reactive group. The linker can be a covalent bond. The linker group may comprise a flexible arm, e.g., 2, 3, 4, 5, 6, 7, 8, 9, 10, 11, 12, 13, 14, or 15 carbon atoms.

Exemplary linkers include, without limitation, pyridinedisulfide, thiosulfonate, vinylsulfonate, isocyanate, imidoester, diazine, hydrazine, thiol, carboxylic acid, multi-peptide linkers, and acetylene. Alternatively other linkers that can be used include BS³ [Bis(sulfosuccinimidyl)suberate] (which is a homobifunctional N-hydroxysuccinimide ester that targets accessible primary amines), NHS/EDC (N-hydroxysuccinimide and 1-ethyl-3-(3-dimethylaminopropyl)carbodiimide (NHS/EDC allows for the conjugation of primary amine groups with carboxyl groups), sulfo-EMCS ([N-ε-maleimidocaproic acid]hydrazide (sulfo-EMCS are heterobifunctional reactive groups that are reactive toward sulfhydryl and amino groups), hydrazide (most proteins contain exposed carbohydrates and hydrazide is a useful reagent for linking carboxyl groups to primary amines).

To form covalent bonds, one can use as a chemically reactive group a wide variety of active carboxyl groups (e.g., esters) where the hydroxyl moiety is physiologically acceptable at the levels required to modify the peptide compound. Particular agents include for example N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA), maleimido hexanoic acid (MHA), and maleimido undecanoic acid (MUA).

Primary amines are the principal targets for NHS esters; NHS esters react with primary amines to form covalent amide bonds. Accessible α-amine groups present on the N-termini of proteins and the ε-amine of lysine react with NHS esters. Thus, conjugated compounds herein disclosed can include a linker having a NHS ester conjugated to an N-terminal amino of a peptide compound, or to an ε-amine of lysine. An amide bond is formed when the NHS ester reacts with primary amines releasing N-hydroxysuccinimide. Succinimide containing reactive groups may be referred to more simply as succinimidyl groups. In some embodiments, the functional group on the peptide compound will be a thiol group and the chemically reactive group will be a maleimido-containing group such as gamma-maleimide-butylamide (GMBA or MPA). Such maleimide-containing groups may be referred to herein as maleido groups.

Amine-to-amine linkers include NHS esters, imidoesters, and others, examples of which are listed below.

Exemplary NHS esters:
DSG (disuccinimidyl glutarate)
DSS (disuccinimidyl suberate)
BS³ (bis[sulfosuccinimidyl] suberate)
TSAT (tris-succinimidyl aminotriacetate)
Variants of bis-succinimide ester-activated
compounds including a polyethylene
glycol spacer such as BS(PEG)ₙ where
n is 1-20 (e.g., BS(PEG)₅ and BS(PEG)₉)
DSP (Dithiobis[succinimidyl propionate])
DTSSP (3,3’-dithiobis[sulfosuccinimidylpropionate])
DST (disuccinimidyl tartarate)
BSOCOES (bis[2-(succinimidooxycarbonyloxy)ethyl]sulfone)
EGS (ethylene glycol bis[succinimidylsuccinate])
sulfo-EGS (ethylene glycol bis[sulfosuccinimidylsuccinate])
Exemplary imidoesters:
DMA (dimethyl adipimidate•2 HCl)
DMP (dimethyl pimelimidate•2 HCl)
DMS (dimethyl suberimidate•2 HCl)
DTBP (dimethyl 3,3‘-dithiobispropionimidate•2 HCl)
Other exemplary amine-to-amine linkers:
DFDNB (1,5-difluoro-2,4-dinitrobenzene)
THPP (β-[tris(hydroxymethyl) phosphino]
propionic acid (betaine))

The linker may also be a sulfhydryl-to-sulfhydryl linker, such as the maleimides and pyridyldithiols listed below.

Exemplary maleimides:
BMOE (bis-maleimidoethane)
BMB (1,4-bismaleimidobutane)
BMH (bismaleimidohexane)
TMEA (tris[2-maleimidoethyl]amine)
BM(PEG)2 1,8-bis-maleimidodiethyleneglycol)
BM(PEG)ₙ, where n is 1 to 20 (e.g., 2 or 3)
BMDB (1,4 bismaleimidyl-2,3-dihydroxybutane)
DTME (dithio-bismaleimidoethane)
Exemplary pyridyldithiol:
DPDPB (1,4-di-[3’-(2’-pyridyldithio)-
propionamido]butane)
Another sulfhydryl linker:
HBVS (1,6-hexane-bis-vinylsulfone)

The linker may be an amine-to-sulfhydryl linker, which includes NHS ester/maleimide compounds. Examples of these compounds are provided below.

Amine-to-sulfhydryl linkers:
AMAS (N-(α-maleimidoacetoxy)succinimide ester)
BMPS (N-[β-maleimidopropyloxy]succinimide ester)
GMBS (N-[γ-maleimidobutyryloxy]succinimide ester)
sulfo-GMBS (N-[γ-maleimidobutyryloxy]sulfosuccinimide ester)
MBS (m-maleimidobenzoyl-N-hydroxysuccinimide ester)
sulfo-MBS (m-maleimidobenzoyl-N-hydroxysulfosuccinimide ester)
SMCC (succinimidyl 4-[N-maleimidomethyl]cyclohexane-1-carboxylate)
sulfo-SMCC (Sulfosuccinimidyl 4-[N-maleimidomethyl]
cyclohexane-1-carboxylate)
EMCS ([N-ε-maleimidocaproyloxy]succinimide ester)
Sulfo-EMCS ([N-ε-maleimidocaproyloxy]sulfosuccinimide ester)
SMPB (succinimidyl 4-[p-maleimidophenyl]butyrate)
sulfo-SMPB (sulfosuccinimidyl 4-[p-maleimidophenyl]butyrate)
SMPH (succinimidyl-6-[β-maleimidopropionamido]hexanoate)
LC-SMCC (succinimidyl-4-[N-maleimidomethyl]cyclohexane-
1-carboxy-[6-amidocaproate])
sulfo-KMUS (N-[κ-maleimidoundecanoyloxy]sulfosuccinimide ester)
SM(PEG)ₙ (succinimidyl-([N-maleimidopropionamido-polyethyleneglycol)
ester), where n is 1 to 30 (e.g., 2, 4, 6, 8, 12, or 24)
SPDP (N-succinimidyl 3-(2-pyridyldithio)-propionate)
LC-SPDP (succinimidyl 6-(3-[2-pyridyldithio]-propionamido)hexanoate)
sulfo-LC-SPDP (sulfosuccinimidyl 6-(3’-[2-pyridyldithio]-
propionamido)hexanoate)
SMPT (4-succinimidyloxycarbonyl-α-methyl-α-[2-pyridyldithio]toluene)
Sulfo-LC-SMPT (4-sulfosuccinimidyl-6-[α-methyl-α-
(2-pyridyldithio)toluamido]hexanoate)
SIA (N-succinimidyl iodoacetate)
SBAP (succinimidyl 3-[bromoacetamido]propionate)
SIAB (N-succinimidyl[4-iodoacetyl]aminobenzoate)
sulfo-SIAB (N-sulfosuccinimidyl[4-iodoacetyl]aminobenzoate)

The linker can react with an amino group and a non-selective entity. Such linkers include NHS ester/aryl azide and NHS ester/diazirine linkers, examples of which are listed below.

NHS ester/aryl azide linkers:
NHS-ASA (N-hydroxysuccinimidyl-4-azidosalicylic acid)
ANB-NOS (N-5-azido-2-nitrobenzoyloxysuccinimide)
sulfo-HSAB (N-hydroxysulfosuccinimidyl-4-azidobenzoate)
sulfo-NHS-LC-ASA (sulfosuccinimidyl[4-azidosalicylamido]hexanoate)
SANPAH (N-succinimidyl-6-(4’-azido-2’-nitrophenylamino)hexanoate)
sulfo-SANPAH (N-sulfosuccinimidyl-6-
(4’-azido-2’-nitrophenylamino)hexanoate)
sulfo-SFAD (sulfosuccinimidyl-(perfluoroazidobenzamido)-
ethyl-1,3’-dithioproprionate)
sulfo-SAND (sulfosuccinimidyl-2-(m-azido-
o-nitrobenzamido)-ethyl-1,3’-proprionate)
sulfo-SAED (sulfosuccinimidyl 2-[7-amino-4-methylcoumarin-
3-acetamido]ethyl-1,3’dithiopropionate)
NHS ester/diazirine linkers:
SDA (succinimidyl 4,4’-azipentanoate)
LC-SDA (succinimidyl 6-(4,4’-azipentanamido)hexanoate)
SDAD (succinimidyl 2-([4,4’-azipentanamido]
ethyl)-1,3’-dithioproprionate)
sulfo-SDA (sulfosuccinimidyl 4,4’-azipentanoate)
sulfo-LC-SDA (sulfosuccinimidyl 6-
(4,4'-azipentanamido)hexanoate)
sulfo-SDAD (sulfosuccinimidyl 2-
([4,4’-azipentanamido]ethyl)-1,3'-dithioproprionate)

Exemplary amine-to-carboxyl linkers include carbodiimide compounds (e.g., DCC (N,N-dicyclohexylcarbodimide) and EDC (1-ethyl-3-[3-dimethylaminopropyl]carbodiimide)). Exemplary sulfhydryl-to-nonselective linkers include pyridyldithiol/aryl azide compounds (e.g., APDP ((N-[4-(p-azidosalicylamido)butyl]-3′-(2′-pyridyldithio)propionamide)). Exemplary sulfhydryl-to-carbohydrate linkers include maleimide/hydrazide compounds (e.g., BMPH (N-[β-maleimidopropionic acid]hydrazide), EMCH ([N-ε-maleimidocaproic acid]hydrazide), MPBH 4-(4-N-maleimidophenyl)butyric acid hydrazide), and KMUH (N-[κ-maleimidoundecanoic acid]hydrazide)) and pyridyldithiol/hydrazide compounds (e.g., PDPH (3-(2-pyridyldithio)propionyl hydrazide)). Exemplary carbohydrate-to-nonselective linkers include hydrazide/aryl azide compounds (e.g., ABH (p-azidobenzoyl hydrazide)). Exemplary hydroxyl-to-sulfhydryl linkers include isocyanate/maleimide compounds (e.g., (N-[p-maleimidophenyl]isocyanate)). Exemplary amine-to-DNA linkers include NHS ester/psoralen compounds (e.g., SPB (succinimidyl-[4-(psoralen-8-yloxy)]-butyrate)).

To generate a branch point of varying complexity in a conjugate peptide compound, the linker can be capable of linking 3-7 entities.

Exemplary tri-functional linkers:

| | | |
|---|---|---|
| TMEA; Tris-(2-maleimidoethyl)amine) | THPP 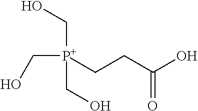 | LC-TSAT (tris-succinimidyl (6- aminocaproyl)aminotriacetate), tris- succinimidyl-1,3,5-benzenetricarboxylate MDSI (maleimido-3,5-disuccinimidyl isophthalate) |
| 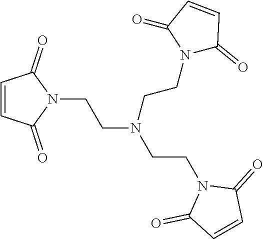 [SMILES: O=C1C=CC(=O)N1CCN(CCN1C(=O)C=CC1=O)CCN1C(=O)C=CC1=O] | | |
| TSAT; Tris-succinimidyl aminotriacetate 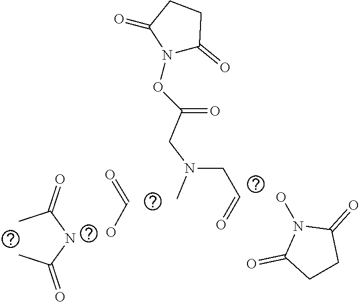 | | SDMB (succinimidyl-3,5- dimaleimidophenyl benzoate Mal-4 (tetrakis-(3-maleimidopropyl) pentaerythritol, NHS-4 (tetrakis-(N- succinimidylcarboxypropyl)pentaerythritol)) |

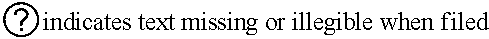

TMEA and TSAT reach through their maleimide groups with sulfhydryl groups. The hydroxyl groups and carboxy group of THPP can react with primary or secondary amines. Other useful linkers conform to the formula Y═C═N-Q-A-C(O)—Z, where Q is a homoaromatic or heteroaromatic ring system; A is a single bond or an unsubstituted or substituted divalent C₁₋₃₀ bridging group, Y is O or S; and Z is Cl, Br, I, N₃, N-succinimidyloxy, imidazolyl, 1-benzotriazolyloxy, OAr where Ar is an electron-deficient activating aryl group, or OC(O)R where R is -A-Q-N═C═Y or C₄-20 tertiary-alkyl (see U.S. Pat. No. 4,680,338).

Other useful linkers have the formula

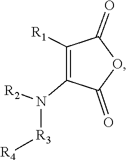

where R₁ is H, C₁₋₆ alkyl, C₂₋₆ alkenyl, C₆₋₁₂ aryl or aralkyl or these coupled with a divalent organic —O—, —S—, or

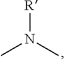

where R′ is C₁₋₆ alkyl, linking moiety; R₂ is H, C₁₋₁₂ alkyl, C₆₋₁₂ aryl, or C₆₋₁₂ aralkyl, R₃ is

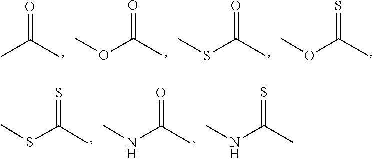

or another chemical structure that is able to delocalize the lone pair electrons of the adjacent nitrogen and R₄ is a pendant reactive group capable of linking R₃ to a peptide compound or to an agent (see for example U.S. Pat. No. 5,306,809).

The linker may include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly or Cys). Where the linker is a short peptide, it can be a glycine-rich peptide (which tend to be flexible) such as a peptide having the sequence [Gly-Gly-Gly-Gly-Ser]ₙ where n is an integer from 1 to 6, inclusive (see U.S. Pat. No. 7,271,149) or a serine-rich peptide linker (see U.S. Pat. No. 5,525,491). Serine rich peptide linkers include those of the formula [X—X—X—X-Gly]_{y} where up to two of the X are Thr, the remaining X are Ser, and y is an integer from 1 to 5, inclusive (e.g., Ser-Ser-Ser-Ser-Gly, where y is greater than 1). Other linkers include rigid linkers (e.g., PAPAP and (PT)ₙP, where n is 2, 3, 4, 5, 6, or 7) and α-helical linkers (e.g., A(EAAAK)ₙA, where n is 1, 2, 3, 4, or 5).

The linker can be an aliphatic linker (e.g., with an amide bond to the polypeptide and an ester bond to the therapeutic agent). Where an aliphatic linker is used, it may vary with regard to length (e.g. C₁-C₂₀) and the chemical moieties it includes (e.g., an amino group or carbamate).

Examples of suitable amino acid linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a NE-acylated lysine residue.

The linker can also be a branched polypeptide. Exemplary branched peptide linkers are described in U.S. Pat. No. 6,759,509, herein incorporated by reference.

The linker can provide a cleavable linkage (e.g., a thioester linkage) or a non-cleavable linkage (e.g., a maleimide linkage). For example, a cytotoxic protein can be bound to a linker that reacts with modified free amines, which are present at lysine residues within the polypeptide and at the amino-terminus of the polypeptide. Thus, linkers useful in the present conjugate compounds can comprise a group that is reactive with a primary amine on the polypeptide or modified polypeptide to which the therapeutic agent moiety is conjugated. More specifically, the linker can be selected from the group consisting of monofluoro cyclooctyne (MFCO), bicyclo[6.1.0]nonyne (BCN), N-succinimidyl-S-acetylthioacetate (SATA), N-succinimidyl-S-acetylthiopropionate (SATP), maleimido and dibenzocyclooctyne ester (a DBCO ester). Useful cyclooctynes, within a given linker, include OCT, ALO, MOFO, DIFO, DIBO, BARAC, DIBAC, and DIMAC.

The linker may comprise a flexible arm, such as for example, a short arm (<2 carbon chain), a medium-size arm (from 2-5 carbon chain), or a long arm (3-6 carbon chain).

Click chemistry can also be used for conjugation on a peptide (DBCO, TCO, tetrazine, azide and alkyne linkers). These families of linkers can be reactive toward amine, carboxyl and sulfhydryl groups. In addition, these linkers can also be biotinylated, pegylated, modified with a fluorescent imaging dye, or phosphoramidited for incorporation onto an oligonucleotide sequence.

The term “intermediate” as used herein refers to a therapeutic agent that has been reacted with a linker thereby forming an intermediate or an activated form of the therapeutic agent. The intermediate can be reacted with a peptide compound herein disclosed thereby forming a conjugate compound herein disclosed that can be used for treatment of a cancer or aggressive cancer.

The expression “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “non-standard,” “unnatural” or “unusual” refers to the wide selection of non-natural, rare or synthetic amino acids such as those described by Hunt, S. in Chemistry and Biochemistry of the Amino Acids, Barrett, G. C., ed., Chapman and Hall: New York, 1985. Some examples of non-standard amino acids include non-alpha amino acids, D-amino acids.

Abbreviations used for amino acids and designation of peptides follow the rules of the IUPAC-IUB Commission of Biochemical Nomenclature in J. Biol. Chem. 1972, 247, 977-983. This document has been updated: Biochem. J., 1984, 219, 345-373; Eur. J. Biochem., 1984, 138, 9-37; 1985, 152, 1; Int. J. Pept. Prot. Res., 1984, 24, following p 84; J. Biol. Chem., 1985, 260, 14-42; Pure Appl. Chem. 1984, 56, 595-624; Amino Acids and Peptides, 1985, 16, 387-410; and in Biochemical Nomenclature and Related Documents, 2^{nd} edition, Portland Press, 1992, pp 39-67. Extensions to the rules were published in the JCBN/NC-IUB Newsletter 1985, 1986, 1989; see Biochemical Nomenclature and Related Documents, 2^{nd} edition, Portland Press, 1992, pp 68-69.

The term “antagonist” refers to a compound that reduces at least some of the effect of the endogenous ligand of a protein, receptor, enzyme, interaction, or the like.

The term “inhibitor” refers to a compound that reduces the normal activity of a protein, receptor, enzyme, interaction, or the like.

The term “library” refers to a collection of compounds that can be used for example for drug discovery purposes. For example, the library compounds can be peptide compounds, or peptide-conjugates herein disclosed.

The term “mixture” as used herein, means a composition comprising two or more peptide-compounds. In an embodiment a mixture is a mixture of two or more distinct peptide-compounds. In a further embodiment, when a peptide-compound is referred to as a “mixture”, this means that it can comprise two or more “forms” of the peptide-compounds, such as, salts, solvates, prodrugs or, where applicable, stereoisomers of the peptide-compound in any ratio. A person of skill in the art would understand that a peptide-compound in a mixture can also exist as a mixture of forms. For example, a peptide-compound may exist as a hydrate of a salt or as a hydrate of a salt of a prodrug of the peptide-compound. All forms of the peptide-compounds herein are within the scope of the present application.

The term “modulator” refers to a peptide-compound that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist.” Exemplary biological processes or mechanisms affected by a modulator include, but are not limited to, enzyme binding, receptor binding and hormone release or secretion. Exemplary chemical processes or mechanisms affected by a modulator include, but are not limited to, catalysis and hydrolysis.

The term “peptide” refers to a chemical compound comprising at least two amino acids covalently bonded together using amide bonds.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The expression “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in Protective Groups in Organic Synthesis, T. W. Greene and P. G. Wuts, eds., John Wiley & Sons, New York, 4^{th} edition, 2006, 1082 pp, ISBN 9780471697541. Examples of amino protecting groups include, but are not limited to, phthalimido, trichloroacetyl, benzyloxycarbonyl, tert butoxycarbonyl, and adamantyl-oxycarbonyl. In some embodiments, amino protecting groups are carbamate amino protecting groups, which are defined as an amino protecting group that when bound to an amino group forms a carbamate. In other embodiments, amino carbamate protecting groups are allyloxycarbonyl (Alloc), benzyloxycarbonyl (Cbz), 9 fluorenylmethoxycarbonyl (Fmoc), tert-butoxycarbonyl (Boc) and α,α dimethyl-3,5 dimethoxybenzyloxycarbonyl (Ddz). For a recent discussion of newer nitrogen protecting groups see: Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to, methyl ester, tert-butyl ester, benzyl ester, trimethylsilylethyl ester, and 2,2,2-trichloroethyl ester.

The expression “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of ref. 52, modified as in ref. 53. Such an algorithm is incorporated into the NBLAST and XBLAST programs of ref. 49. BLAST nucleotide searches can be performed with the NBLAST nucleotide program parameters set, e.g., for score=100, wordlength=12 to obtain nucleotide sequences homologous to a nucleic acid molecule of the present application. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in ref. 47. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The expression “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The expression “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry (Solid-Phase Synthesis: A Practical Guide, F. Albericio, ed., CRC Press, 2000, 848 pp, ISBN: 978-0824703592; Organic Synthesis on Solid Phase, 2^{nd} edition, Florencio Zaragoza Dörwald, Wiley-VCH, 2002, 530 pp, ISBN: 3-527-30603-X; Solid-Phase Organic Synthesis: Concepts, Strategies, and Applications, P. H. Toy, Y. Lam, eds., Wiley, 2012, 568 pp, ISBN: 978-0470599143).

The term “solid support,” “solid phase” or “resin” refers to a mechanically and chemically stable polymeric matrix utilized to conduct solid phase chemistry. This is denoted by “Resin,” “P-” or the following symbol: [symbol] .

Examples of appropriate polymer materials include, but are not limited to, polystyrene, polyethylene, polyethylene glycol (PEG, including, but not limited to, ChemMatrix® (Matrix Innovation, Quebec, Quebec, Canada; J. Comb. Chem. 2006, 8, 213-220)), polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., ed.; SPCC Ltd.: Birmingham, UK; p 205), polyacrylate (CLEAR™), polyacrylamide, polyurethane, PEGA [polyethyleneglycol poly(N,N dimethyl-acrylamide) co-polymer, Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, preferably 0.5-2%). This solid support can include as non-limiting examples aminomethyl polystyrene, hydroxymethyl polystyrene, benzhydrylamine polystyrene (BHA), methylbenzhydrylamine (MBHA) polystyrene, and other polymeric backbones containing free chemical functional groups, most typically, NH₂ or —OH, for further derivatization or reaction. The term is also meant to include “Ultraresins” with a high proportion (“loading”) of these functional groups such as those prepared from polyethyleneimines and cross-linking molecules (J. Comb. Chem. 2004, 6, 340-349). At the conclusion of the synthesis, resins are typically discarded, although they have been shown to be able to be recycled (Tetrahedron Lett. 1975, 16, 3055).

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The expression “pharmaceutically acceptable” means compatible with the treatment of subjects such as animals or humans.

The expression “pharmaceutically acceptable salt” means an acid addition salt or basic addition salt which is suitable for or compatible with the treatment of subjects such as animals or humans.

The expression “pharmaceutically acceptable acid addition salt” as used herein means any non-toxic organic or inorganic salt of any compound of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, formic, acetic, malic, tartaric, citric, ascorbic, maleic, benzoic, phenylacetic, cinnamic and salicylic acids, as well as sulfonic acids such as p-toluenesulfonic and methanesulfonic acids. Either the mono or di-acid salts can be formed, and such salts may exist in either a hydrated, solvated or substantially anhydrous form. In general, the acid addition salts of the compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the present disclosure, for laboratory use, or for subsequent conversion to a pharmaceutically acceptable acid addition salt.

The expression “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where CO₂H is a functional group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds, or conjugate compounds of the disclosure, for laboratory use, or for subsequent conversion to a pharmaceutically acceptable acid addition salt.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound, or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals such as a mouse, a rat, a dog and a human.

The terms “suitable” and “appropriate” mean that the selection of the particular group or conditions would depend on the specific synthetic manipulation to be performed and the identity of the molecule but the selection would be well within the skill of a person trained in the art. All process steps described herein are to be conducted under conditions suitable to provide the product shown. A person skilled in the art would understand that all reaction conditions, including, for example, reaction solvent, reaction time, reaction temperature, reaction pressure, reactant ratio and whether the reaction should be performed under an anhydrous or inert atmosphere, can be varied to optimize the yield of the desired product and it is within their skill to do so.

The expression a “therapeutically effective amount”, “effective amount” or a “sufficient amount” of a compound or composition of the present disclosure is a quantity sufficient to, when administered to the subject, including a mammal, for example a human, effect beneficial or desired results, including clinical results, and, as such, a “therapeutically effective amount” or an “effective amount” depends upon the context in which it is being applied. For example, in the context of treating cancer, it is an amount of the compound, peptide compound-conjugate, or composition sufficient to achieve such treatment of the cancer as compared to the response obtained without administration of the compound, peptide compound-conjugate, or composition. The amount of a given compound, peptide compound-conjugate, or composition of the present disclosure that will correspond to an effective amount will vary depending upon various factors, such as the given drug, peptide compound-conjugate, the pharmaceutical formulation, the route of administration, the type of disease or disorder, the identity of the subject or host being treated, and the like, but can nevertheless be routinely determined by one skilled in the art. Also, as used herein, a “therapeutically effective amount” or “effective amount” of a compound, peptide compound-conjugate, or composition of the present disclosure is an amount which inhibits, suppresses or reduces a cancer (e.g., as determined by clinical symptoms or the amount of cancerous cells) in a subject as compared to a control.

As used herein, and as well understood in the art, “treatment” or “treating” is an approach for obtaining beneficial or desired results, including clinical results. Beneficial or desired clinical results can include, but are not limited to, decrease in tumour progression, decrease in tumour size, decrease in tumour growth rate, decrease in tumor invasion and metastatic potential, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” or “treating” can also mean prolonging survival as compared to expected survival if not receiving treatment.

The term “tolerability” or “tolerated” as used herein means a degree to which a therapeutic agent may be endured or accepted by a subject treated with the therapeutic agent. For example, tolerability may be assessed by measuring different parameters such as (i) maintenance or absence of weight loss, (ii) duration of treatment withstood and (iii) decrease or absence of side effects such as for example neutropenia. For example, it is well established that a therapeutic agent is tolerated by a subject when there is no weight loss observed during treatment using such a therapeutic agent. For example, the conjugates of the present disclosure (comprising at least one therapeutic agent) can increase the tolerability of a given therapeutic agent since the conjugate is being more selective to receptors than the therapeutic agent taken alone. Unconjugated toxins may be too toxic to be administered or used alone in a subject. Therefore, highly potent toxins can be used in drug conjugates to increase tolerability. In some embodiments, the therapeutic agent is a toxin selected from the group consisting of maytansinoids, auristatins, calicheamicins, amatoxin, and amanitin.

The term “administered” or “administering” as used herein means administration of a therapeutically effective amount of a compound, peptide compound-conjugate, or composition of the application to a cell either in vitro (e.g. a cell culture) or in vivo (e.g. in a subject).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In compositions comprising an “additional” or “second” component, the second component as used herein is chemically different from the other components or first component. A “third” component is different from the other, first, and second components, and further enumerated or “additional” components are similarly different.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

A platform allowing the transport of therapeutic agents into cancer cells for new therapies directed against primary and secondary tumours was previously developed. This approach utilizes peptide compounds derived from bacterial proteins or from ligands of receptors expressed in cancer cells (e.g. sortilins/syndecans).

Disclosed herein are compositions comprising a solubilizing agent and peptide compounds as well as compositions comprising a solubilizing agent and conjugate compounds comprising at least one therapeutic agent connected to a peptide compound for use treating a cancer.

Accordingly, a first aspect is a peptide compound having at least 60% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | (XIII) |
| (SEQ ID NO: 13) | |
| | YKSLRRKAPRWDAYLRDPALRPLL |

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L; n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end.

Another aspect is a composition comprising a solubilizing agent and a peptide compound having at least at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | (XIII) |
| (SEQ ID NO: 13) | |
| | YKSLRRKAPRWDAYLRDPALRPLL |

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end.

Yet another aspect is a composition comprising a solubilizing agent and a peptide compound having at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | (XIII) |
| (SEQ ID NO: 13) | |
| | YKSLRRKAPRWDAYLRDPALRPLL |

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end.

In some embodiments, the peptide compound targets Sortilin receptor. In some embodiments, the peptide compound is for use in targeting Sortilin receptor.

For example, the peptide compound is a peptide compound that comprises:

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | or |
| | (XIII) |
| (SEQ ID NO: 13) | |
| | YKSLRRKAPRWDAYLRDPALRPLL. |

For example, the peptide compound is a peptide compound that consists essentially of:

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWVDX₁₅PLRDPALRX₁₆X₁₇ |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | or |
| | (XIII) |
| (SEQ ID NO: 13) | |
| | YKSLRRKAPRWDAYLRDPALRPLL. |

For example, the peptide compound is a peptide compound that consists of:

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWDX₁₅PLRDPALRX₁₆X₁₇L |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | or |
| | (XIII) |
| (SEQ ID NO: 13) | |
| | YKSLRRKAPRWDAYLRDPALRPLL. |

For example, the peptide compound comprises a compound chosen from compounds of formula (I), formula (II), formula (III), formula (IV), formula (V), formula (VI), formula (VII), formula (VIII), formula (IX), formula (X), formula (XI), formula (XII) and formula (XIII):

| | (I) |
|---|---|
| (SEQ ID NO: 1) | |
| | X₁X₂X₃X₄X₅GVX₆AKAGVX₇NX₈FKSESY |
| | (II) |
| (SEQ ID NO: 2) | |
| | (X₉)ₙGVX₁₀AKAGVX₁₁NX₁₂FKSESY |
| | (III) |
| (SEQ ID NO: 3) | |
| | YKX₁₃LRRX₁₄APRWVDX₁₅PLRDPALRX₁₆X₁₇ |
| | (IV) |
| (SEQ ID NO: 4) | |
| | YKX₁₈LRR(X₁₉)ₙPLRDPALRX₂₀X₂₁L |
| | (V) |
| (SEQ ID NO: 5) | |
| | IKLSGGVQAKAGVINMDKSESM |
| | (VI) |
| (SEQ ID NO: 6) | |
| | IKLSGGVQAKAGVINMFKSESY |
| | (VII) |
| (SEQ ID NO: 7) | |
| | IKLSGGVQAKAGVINMFKSESYK |
| | (VIII) |
| (SEQ ID NO: 8) | |
| | GVQAKAGVINMFKSESY |
| | (IX) |
| (SEQ ID NO: 9) | |
| | GVRAKAGVRNMFKSESY |
| | (X) |
| (SEQ ID NO: 10) | |
| | GVRAKAGVRN(Nle)FKSESY |
| | (XI) |
| (SEQ ID NO: 11) | |
| | YKSLRRKAPRWDAPLRDPALRQLL |
| | (XII) |
| (SEQ ID NO: 12) | |
| | YKSLRRKAPRWDAYLRDPALRQLL |
| | (XIII) |
| (SEQ ID NO: 13) | |
| | YKSLRRKAPRWDAYLRDPALRPLL |

wherein
X₁, X₂, X₃, X₄, X₅, X₆, X₇, X₈, X₉, X₁₀, X₁₁, X₁₂, X₁₃, X₁₄, X₁₅, X₁₈ and X₁s are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound chosen from peptide compounds of formula (I), formula (II), formula (III), formula (IV), formula (V), formula (VI), formula (VII), formula (VIII), formula (IX), formula (X), formula (XI), formula (XII) and formula (XIII).

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (I) or SEQ ID NO: 1.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (II) or SEQ ID NO: 2.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (III) or SEQ ID NO: 3.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IV) or SEQ ID NO: 4.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (V) or SEQ ID NO: 5.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VI) or SEQ ID NO: 6.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VII) or SEQ ID NO: 7.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VIII) or SEQ ID NO: 8.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IX) or SEQ ID NO: 9.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (X) or SEQ ID NO: 10.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XI) or SEQ ID NO: 11.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XII) or SEQ ID NO: 12.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XIII) or SEQ ID NO: 13.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (LI) or SEQ ID NO: 23.

In one embodiment, n is 0. In one embodiment, n is 1. In one embodiment, n is 2. In one embodiment, n is 3. In one embodiment, n is 4. In one embodiment, n is 5.

In an embodiment, the peptide compound is represented by formula (I) or formula (II). In one embodiment, the peptide compound is represented by formula (I) or SEQ ID NO: 1. In one embodiment, the peptide compound is represented by formula (II) or SEQ ID NO: 2. In one embodiment, the peptide compound is represented by formula (III) or formula (IV). In one embodiment, the peptide compound is represented by formula (III). In one embodiment, the peptide compound is represented by formula (IV). In an embodiment, the peptide compound is represented by formula (V), formula (VI), formula (VII), formula (VIII), formula (IX) or formula (X). In one embodiment, the peptide compound is represented by formula (V). In one embodiment, the peptide compound is represented by formula (VI). In one embodiment, the peptide compound is represented by formula (VII). In one embodiment, the peptide compound is represented by formula (VIII). In one embodiment, the peptide compound is represented by formula (IX). In one embodiment, the peptide compound is represented by formula (X). In one embodiment, the peptide compound is represented by formula (XI), formula (XII) or formula (XIII). In one embodiment, the peptide compound is represented by formula (XI). In one embodiment, the peptide compound is represented by formula (XII). In one embodiment, the peptide compound is represented by formula (XIII). In one embodiment, the peptide compound is represented by formula (LI).

In one embodiment, the peptide compound is represented by the amino acid sequence of SEQ ID NO: 1. In one embodiment, the peptide compound is represented by the amino acid sequence of SEQ ID NO: 2. In one embodiment, the peptide compound is represented by the amino acid sequence of SEQ ID NO: 3. In one embodiment, the peptide compound is represented by the amino acid sequence of SEQ ID NO: 4. In one embodiment, the peptide compound is represented by the amino acid sequence of SEQ ID NO: 5. In one embodiment, the peptide compound is represented by the amino acid sequence of SEQ ID NO: 6. In one embodiment, the peptide compound is represented by the amino acid sequence of SEQ ID NO: 7. In one embodiment, the peptide compound is represented by the amino acid sequence of SEQ ID NO: 8. In one embodiment, the peptide compound is represented by the amino acid sequence of SEQ ID NO: 9. In one embodiment, the peptide compound is represented by the amino acid sequence of SEQ ID NO: 10. In one embodiment, the peptide compound is represented by the amino acid sequence of SEQ ID NO: 11. In one embodiment, the peptide compound is represented by the amino acid sequence of SEQ ID NO: 12. In one embodiment, the peptide compound is represented by the amino acid sequence of SEQ ID NO: 13. In one embodiment, the peptide compound is represented by the amino acid sequence of SEQ ID NO: 23.

In one embodiment, at least one protecting group is connected to said peptide at an N- and/or C-terminal end.

In one embodiment, a succinyl group is connected to the peptide compound. For example, the peptide compound has the sequence of Succinyl-IKLSGGVQAKAGVINMFKSESY, corresponding to SEQ ID NO: 6 and having a succinyl group attached thereto at the N-terminal end.

In one embodiment, an acetyl group is connected to the peptide compound. For example, the peptide compound has the sequence of Acetyl-GVRAKAGVRNMFKSESY (SEQ ID NO: 14). For example, the peptide compound has the sequence of Acetyl-GVRAKAGVRN(Nle)FKSESY (SEQ ID NO: 15). For example, the peptide compound has the sequence of Acetyl-YKSLRRKAPRWDAPLRDPALRQLL (SEQ ID NO: 16). For example, the peptide compound has the sequence of Acetyl-YKSLRRKAPRWDAYLRDPALRQLL (SEQ ID NO: 17). For example, the peptide compound has the sequence of Acetyl-YKSLRRKAPRWDAYLRDPALRPLL (SEQ ID NO: 18).

In one embodiment, at least one labelling agent is connected to said peptide at an N- and/or C-terminal end.

The person skilled in the art will understand that commonly used labelling agents can be used. For example, the labelling agent is a vitamin. For example, the labelling agent is biotin. For example, the labelling agent is used as a fluorescent probe and/or as an imaging agent.

In one embodiment, the peptide compound is biotinylated. For example, the peptide compound has the sequence of IKLSGGVQAKAGVINMFKSESYK(Biotin), corresponding to SEQ ID NO: 7 and having a biotin molecule attached thereto at the C-terminal end.

For example, the peptide compound is represented by Formula (XXXVI):

Succinyl-IKLSGGVQAKAGVINMFKSESY (XXXVI)

that comprises the peptide compound having SEQ ID NO: 6 wherein a succinyl group is attached at the N-terminal end.

In one embodiment, X₁₆ is independently chosen from Q, P, Y, I and L.

For example, X₁₆ is Q. For example, X₁₆ is P. For example, X₁₆ is Y. For example, X₁₆ is I.

In one embodiment, X₁₇ is independently chosen from Q, P, Y, I and L.

For example, X₁₇ is Q. For example, X₁₇ is P. For example, X₁₇ is Y. For example, X₁₇ is I.

In one embodiment, X₂₀ is independently chosen from Q, P, Y, I and L.

For example, X₂₀ is Q. For example, X₂₀ is P. For example, X₂₀ is Y. For example, X₂₀ is I.

In one embodiment, X₂₁ is independently chosen from Q, P, Y, I and L.

For example, X₂₁ is Q. For example, X₂₁ is P. For example, X₂₁ is Y. For example, X₂₁ is I.

In one embodiment, the peptide compound is chosen from:

| (SEQ ID NO: 1) | |
|---|---|
| | X₁X₂X₃X₄X₅GVX₆AKAGVX₇NX₈FKSESY; |
| (SEQ ID NO: 2) | |
| | (X₉)ₙGVX₁₀AKAGVX₁₁NX₁₂FKSESY; |
| (SEQ ID NO: 3) | |
| | YKX₁₃LRRX₁₄APRWDX₁₅PLRDPALRX₁₆X₁₇L; |
| (SEQ ID NO: 4) | |
| | YKX₁₈LRR(X₁₉)ₙPLRDPALRX₂₀X₂₁L; |
| (SEQ ID NO: 5) | |
| | IKLSGGVQAKAGVINMDKSESM; |
| | Succinyl-IKLSGGVQAKAGVINMFKSESY |
| | (that comprises SEQ ID NO: 6 |
| | wherein a succinyl group is |
| | attached thereto at the |
| | N-terminal end); |
| | IKLSGGVQAKAGVINMFKSESYK(Biotin) |
| | (that comprises SEQ ID NO: 7 |
| | wherein a biotin molecule is |
| | attached thereto at the |
| | C-terminal end); |
| (SEQ ID NO: 8) | |
| | GVQAKAGVINMFKSESY; |
| (SEQ ID NO: 14) | |
| | Acetyl-GVRAKAGVRNMFKSESY; |
| (SEQ ID NO: 15) | |
| | Acetyl-GVRAKAGVRN(Nle)FKSESY; |
| (SEQ ID NO: 16) | |
| | Acetyl-YKSLRRKAPRWDAPLRDPALRQLL; |
| (SEQ ID NO: 17) | |
| | Acetyl-YKSLRRKAPRWDAYLRDPALRQLL; |
| (SEQ ID NO: 18) | |
| | Acetyl-YKSLRRKAPRWDAYLRDPALRPLL; |
| (SEQ ID NO: 23) | |
| | GVRAKAGVRN(Nle)FKSESYC; |
| | and |
| (SEQ ID NO: 24) | |
| | Acetyl-GVRAKAGVRN(Nle)FKSESYC. |

In one embodiment, the peptide compounds can be modified at the C- and/or N-terminal by the addition of one or more amino acid residue in order to obtain or increase preferential binding sites at the peptide terminal end. For example, the amino acid can be cysteine. For example, the amino acid can be lysine. For example, the amino acid can be cysteine added at the C-terminal of a peptide. In one embodiment, the peptide compound is modified by the addition of cysteine at the C-terminal. In a specific embodiment, the peptide compound has the sequence of Acetyl-GVRAKAGVRN(Nle)FKSESY corresponding to SEQ ID NO: 15 is modified by the addition of cysteine at the C-terminal.

The peptide compounds described herein can be connected, linked, mixed or conjugated to small molecules, peptides, proteins, oligonucleotides, diagnostic agents, imaging or radionuclide agents, large molecules such as monoclonal antibodies, therapeutic agents such phytochemicals or to drug delivery systems including nanoparticles, liposomes, nanotubes, graphene particles loaded with a therapeutic agent, imaging agent, gene, siRNA. The resulting conjugate compounds can be used as mono- or combined therapies for example for treating a cancer.

Accordingly, another aspect disclosed herein is a conjugate compound having the formula of A-(B)ₙ,

wherein
n is 1, 2, 3 or 4;
A is a peptide compound as defined herein, wherein said peptide is optionally protected by a protecting group; and
B is at least one therapeutic agent, wherein B is connected to A,
optionally the peptide compound is cyclic.

Yet another aspect disclosed herein is a conjugate compound having the formula of A-(B)ₙ,

wherein
n is 1, 2, 3 or 4;
A is a peptide compound as defined in the present disclosure, wherein said peptide compound is optionally protected by a protecting group; and
B is at least one therapeutic agent, wherein B is connected to A, optionally at a free amine of said peptide compound, at an N-terminal position of said peptide compound, at a free —SH of said peptide compound, or at a free carboxyl of said peptide compound,
optionally the peptide compound is cyclic.

In some embodiments, the conjugate peptide described herein targets Sortilin receptor. In some embodiments, the conjugate peptide described herein is for use in targeting Sortilin receptor.

Yet another aspect disclosed herein is a conjugate compound having the formula of A-(B)ₙ,

wherein
n is 1, 2, 3 or 4;
A is a peptide compound as defined herein; and
B is at least one therapeutic agent, wherein B is connected to A at a free amine of a lysine residue of said peptide compound, optionally via a linker, or at an N-terminal position of said peptide compound, optionally via a linker,
optionally the peptide compound is cyclic,
for use in treating cancer or aggressive cancer.

In an embodiment, B is connected to A via a linker, optionally a cleavable linker.

For example, the at least one therapeutic agent is a phytochemical chosen from curcumin, omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

In an embodiment, the therapeutic agent is a phytochemical or an anticancer agent.

In an embodiment, the phytochemical is curcumin.

In an embodiment, the conjugate compound is chosen from:

GVRAK(curcumin)AGVRN(Nle)FK(curcumin)SESY Formula (XIV)

that comprises the peptide compound having SEQ ID NO: 10 wherein each lysine residue has a curcumin molecule connected thereto; and

YK(curcumin)SLRRK(curcumin)APRWDAPLRDPALRQLL Formula (XV)

that comprises the peptide compound having SEQ ID NO: 11 wherein each lysine residue has a curcumin molecule connected thereto.

For example, the conjugate compound is represented by formula (XIV).

For example, the conjugate compound is represented by formula (XV).

In an embodiment, the conjugate compound is chosen from:

Acetyl-GVRAK(curcumin)AGVRN(Nle)FK(curcumin)SESY Formula (XVI)

that comprises the peptide compound having SEQ ID NO: 15 wherein each lysine residue has a curcumin molecule connected thereto, and

Acetyl-YK(curcumin)SLRRK(curcumin)APRWDAPLRDPALRQLL Formula (XVII)

that comprises the peptide compound having SEQ ID NO: 16 wherein each lysine residue has a curcumin molecule connected thereto.

For example, the conjugate compound is represented by formula (XVI).

For example, the conjugate compound is represented by formula (XVII).

In an embodiment, the therapeutic agent is an anticancer agent.

In an embodiment, the anticancer agent is docetaxel.

In an embodiment, the conjugate compound is represented by formula (XIX):

GVRAK(docetaxel)AGVRN(Nle)FK(docetaxel)SESY Formula (XIX)

that comprises the peptide compound having SEQ ID NO: 10 wherein each lysine residue has a docetaxel molecule connected thereto.

In another embodiment, the conjugate compound is represented by formula (XXIII):

Acetyl-GVRAK(docetaxel)AGVRN(Nle)FK(docetaxel)SESY Formula (XXIII)

that comprises the peptide compound having SEQ ID NO: 15 wherein each lysine residue has a docetaxel molecule connected thereto.

In an embodiment, the anticancer agent is doxorubicin.

In an embodiment, the conjugate compound is represented by formula (XXVI):

GVRAK(doxorubicin)AGVRN(Nle)FK(doxorubicin)SESY Formula (XXVI)

that comprises the peptide compound having SEQ ID NO: 10 wherein each lysine residue has a doxorubicin molecule connected thereto.

In another embodiment, the conjugate compound is represented by formula (XXVIII):

Acetyl-GVRAK(doxorubicin)AGVRN(Nle)FK(doxorubicin)SESY Formula (XXVIII)

that comprises the peptide compound having SEQ ID NO: 15 wherein each lysine residue has a doxorubicin molecule connected thereto.

In an embodiment, the anticancer agent is cabazitaxel.

In an embodiment, the anticancer agent is aldoxorubicin.

In an embodiment, the conjugate compound is represented by formula (LI):

GVRAKAGVRN(Nle)FKSESYC(aldoxorubicin) Formula (LI)

that comprises the peptide compound having SEQ ID NO: 23 wherein cysteine residue has an aldoxorubicin molecule connected thereto, or
that comprises the peptide compound having SEQ ID NO: 10 wherein a cysteine residue is added to C-terminal of said peptide compound, and wherein the cysteine residue has an aldoxorubicin molecule connected thereto.

In an embodiment, the conjugate compound is represented by formula (LII):

Acetyl-GVRAKAGVRN(Nle)FKSESYC(aldoxorubicin) Formula (LII)

that comprises the peptide compound having SEQ ID NO: 24 wherein cysteine residue has an aldoxorubicin molecule connected thereto, or
that comprises the peptide compound having SEQ ID NO: 15 wherein a cysteine residue is added to C-terminal of said peptide compound, and wherein the cysteine residue has an aldoxorubicin molecule connected thereto.

In an embodiment, B, the at least one therapeutic agent, is connected to A, the peptide compound, at said free amine of said lysine residue of said peptide compound, via a linker.

In an embodiment, B, the at least one therapeutic agent, is connected to A, the peptide compound, at said N-terminal position of said peptide compound, via a linker.

In an embodiment, the linker is chosen from succinic acid and dimethyl glutaric acid linker.

For example, the linker is a cleavable linker. For example, the linker is a non-cleavable linker.

For example, the conjugate compound can comprise a cleavable linker connected the at least one therapeutic agent to the peptide compound. For example, the at least one therapeutic agent can be released from the peptide compound by the action of esterases on the ester bond.

For example, a therapeutic agent can be conjugated to the peptide compound on free amines available on the peptide, at the lysine or amino-terminal, by forming a bond such as a peptide bond.

In an embodiment, wherein B is connected to A′ via a linker, optionally a cleavable linker or a non-cleavable linker. In an embodiment, the at least one therapeutic agent is an anticancer agent. In an embodiment, the anticancer agent docetaxel, doxorubicin, cabazitaxel, aldoxorubicin, maytansinoids, auristatins, calicheamicins, amatoxins, amanitin and oligopeptidomimetics (e.g. tubulysins). In an embodiment, the at least one therapeutic agent is a phytochemical, optionally curcumin. In an embodiment, the anticancer agent is docetaxel. In an embodiment, the anticancer agent is doxorubicin. In an embodiment, the anticancer agent is cabazitaxel. In an embodiment, the anticancer agent is aldoxorubicin.

In an embodiment, the conjugate compound comprises 1 molecule of the therapeutic agent connected to the peptide compound. In an embodiment, the conjugate compound comprises at least 1 molecule of the therapeutic agent connected to the peptide compound. In an embodiment, the conjugate compound comprises up to 8 molecules of the therapeutic agent connected to the peptide compound

In an embodiment, the conjugate compound or comprises 2 molecules of the therapeutic agent connected to the peptide compound. In an embodiment, the conjugate compound comprises 3 molecules of the therapeutic agent connected to the peptide compound. In an embodiment, the conjugate compound comprises 4 molecules of the therapeutic agent connected to the peptide compound. In an embodiment, the conjugate compound comprises 1-8 molecules of the therapeutic agent connected to the peptide compound

In an aspect, the compound described herein targets Sortilin receptor. In an aspect, the compound described herein is for use in targeting Sortilin receptor.

In an aspect, the compound described herein is for treatment of cancer or aggressive cancer.

For example, the cells expressing Sortilin are immune cells, optionally macrophages, CD4+, CD8+, B220+, bone marrow-derived cells basophils, eosinophils and cytotoxic T lymphocytes, Natural Killer (NK) cells, T helper type 1 (Th1) cells.

For example, the cells expressing Sortilin are cancer cells, optionally ovarian cancer cells, endometrial cancer cells, breast cancer cells (e.g. triple negative breast cancer cells, optionally HCC1599, HCC1937, HCC1143, MDA-MB468, HCC38, HCC70, HCC1806, HCC1187, DU4475, BT-549, Hs578T, MDA-MB231, MDA-MB436, MDA-MB157, MDA-MB453, BT-20, or HCC1395 cells), prostate cancer cells, colorectal cancer cells, lung cancer cells, pancreas cancer cells, skin cancer cells, brain (gliomas) cancer cells, urothelial cancer cells, carcinoid cancer cells, renal cancer cells, testis cancer cells, pituitary cancer cells and blood cancer cells such as bone marrow cancer cells, diffuse large B cell lymphoma cancer cells, myeloma cancer cells or chronic B cell leukemia cancer cells.

For example, the cells expressing Sortilin are cancer cells, optionally ovarian cancer cells, endometrial cancer cells, breast cancer cells (e.g. triple negative breast cancer cells), prostate cancer cells, colorectal cancer cells, lung cancer cells, pancreas cancer cells, skin cancer cells, brain (gliomas) cancer cells, urothelial cancer cells, carcinoid cancer cells, renal cancer cells, testis cancer cells, pituitary cancer cells and blood cancer cells such as bone marrow cancer cells, diffuse large B cell lymphoma cancer cells, myeloma cancer cells or chronic B cell leukemia cancer cells.

For example, the triple negative breast cancer cells are HCC1599, HCC1937, HCC1143, MDA-MB468, HCC38, HCC70, HCC1806, HCC1187, DU4475, BT-549, Hs578T, MDA-MB231, MDA-MB436, MDA-MB157, MDA-MB453, BT-20, or HCC1395 cells.

Conjugate compound conjugate herein disclosed can also be used to transport therapeutic agents into the cell as they are not a substrate of efflux pumps such as the P-glycoprotein membrane transporter pump which pumps out other therapeutic agents from multi resistant drug cells.

In an aspect, there is provided a method for obtaining the peptide compound, comprising i) providing a library of binder peptides; and ii) selecting sortilin-binding peptides from the library by means of affinity selection using a target;

wherein the target is immobilized on a solid support;

wherein the target is comprised of amino acid sequence as shown in any one of SEQ ID NOs: 25-50, an analog thereof, or a fragment thereof; and

wherein the target is in interaction with the sortilin-binding peptide.

In a further aspect, there is provided a process for preparing conjugate compound conjugate herein disclosed, the process comprising:

reacting a linker together with said therapeutic agent so as to obtain an intermediate;
optionally purifying said intermediate;
reacting said intermediate together with peptide compound so as to obtain said conjugate compound; and
optionally purifying said conjugate compound conjugate;
wherein the therapeutic agent is connected to the peptide compound at a free amine of a lysine residue or an N-terminal; and wherein the peptide compound comprises 1, 2, 3 or 4 therapeutic agent molecules connected thereto.

For example, the peptide compound comprises 1 therapeutic agent molecule connected thereto. For example, the peptide compound comprises 2 therapeutic agent molecules connected thereto. For example, the peptide compound comprises 3 therapeutic agent molecules connected thereto. For example, the peptide compound comprises 4 therapeutic agent molecules connected thereto.

For example, the linker is succinic acid. For example, the linker is a dimethyl glutaric acid linker.

In an embodiment, the peptide compound is protected at said N-terminal prior to reacting with said intermediate.

For example, a protecting group such as FMOC can be added as a protecting group to a free amine on the therapeutic agent prior to incorporation with a linker. After its synthesis, the conjugate compound can undergo deprotection from the protecting group. For example, the conjugate compound comprising the protecting agent FMOC can be deprotected using piperidin. The person skilled in the art would readily understand that other known chemical reagents may be used for deprotection of conjugate compounds.

For example, the N-terminal of the therapeutic agent, the peptide compound can be capped by its acetylation, thereby providing a non-reversible protecting group at the N-terminal.

In an embodiment, the intermediate is activated prior to reacting with said peptide compound.

For example, the intermediate is activated prior to reacting with said compound with a coupling agent, optionally chosen from N,N,N′,N′-Tetramethyl-O-(benzotriazol-1-yl)uronium tetrafluoroborate (TBTU), (2-(1H-benzotriazol-1-yl)-1,1,3,3-tetramethyluronium hexafluorophosphate) (HBTU), and (1-[Bis(dimethylamino)methylene]-1H-1,2,3-triazolo[4,5-b]pyridinium 3-oxid hexafluorophosphate) (HATU).

For example, the intermediate comprising a therapeutic agent connected to a linker can be activated with TBTU, a peptide coupling reagent, prior to conjugation with the peptide compound.

In one embodiment, the conjugate compound is purified following its synthesis.

Peptide compounds disclosed herein are useful in the context of fusion proteins. For example, a fusion protein can be engineered by fusing a peptide compound herein disclosed, for example a peptide compound, to one or more proteins, or parts thereof such as functional domains. Fusion proteins can be engineered for example by recombinant DNA technology and expressed using a protein expression system such as a bacterial or mammalian protein expression system. In some embodiments, peptide linkers are added between proteins. In other embodiment, the fusion proteins do not comprise linkers connecting the proteins.

Commonly used protein expression systems include those derived from bacteria, yeast, baculovirus/insect, plants and mammalian cells and more recently filamentous fungi such as the Myceliophthora thermophile.

An aspect herein disclosed is a composition comprising a solubilizing agent and a liposome, graphene, nanotube or nanoparticle comprising at least one peptide compound disclosed herein for use in treating a cancer.

An aspect herein disclosed is a composition comprising a solubilizing agent and liposome, graphene, nanotube or nanoparticle comprising at least one peptide compound disclosed herein that targets Sortilin receptor.

An aspect herein disclosed is a composition comprising a solubilizing agent and liposome, graphene, nanotube or nanoparticle comprising at least one peptide compound disclosed herein for use in targeting Sortilin receptor.

Another aspect is a composition comprising a solubilizing agent and liposome, graphene, nanotube or nanoparticle coated with at least one compound disclosed herein for use in treating a cancer.

Another aspect is a composition comprising a solubilizing agent and a liposome, graphene, nanotube or nanoparticle coated with at least one compound disclosed herein that targets Sortilin receptor.

Another aspect is a composition comprising a solubilizing agent and a liposome, graphene, nanotube or nanoparticle coated with at least one compound disclosed herein for use in targeting Sortilin receptor.

Another aspect is a composition comprising a solubilizing agent and a liposome, graphene, nanotube or nanoparticle loaded with at least one therapeutic agent, gene or siRNA; and the liposome or nanoparticle is coated with at least one compound herein defined, for use in treating a cancer.

Different embodiments of liposomes, nanotubes, graphene or nanoparticles can be envisaged by the person skilled in the art. For example the liposome or nanoparticle can comprise at least one compound herein disclosed coated on the surface of the liposome or nanoparticle and a therapeutic agent, for example an anticancer agent, within the liposome or nanoparticle. For example, the liposome or nanoparticle can comprise at least one compound herein disclosed coated on the surface of the liposome or nanoparticle and a therapeutic agent, for example an anticancer agent, within the liposome or nanoparticle. For example, the liposome or nanoparticle can comprise at least one peptide compound herein disclosed coated on the surface of the liposome or nanoparticle and a therapeutic agent, for example an anticancer agent, within the liposome or nanoparticle. For example, the liposome or nanoparticle can comprise at least one conjugate compound herein disclosed coated on the surface of the liposome or nanoparticle and a therapeutic agent, for example an anticancer agent, within the liposome or nanoparticle. For example, the liposome or nanoparticle can comprise at least one compound herein disclosed coated on the surface of the liposome or nanoparticle and a therapeutic agent, for example an anticancer agent, within the liposome or nanoparticle. In addition, in some embodiments, the compound herein described can be associated, linked, or connected to one or more other compounds to form a multimer such as a dimer, a trimer or a tetramer, as well as branched peptides, optionally the peptide compound is cyclic. Such compound can be connected together, for example via a covalent bond, an atom or a linker. For example, the multimer comprises more than one compound. Methods for making multimeric (e.g. dimeric, trimeric) forms of peptide compounds are described in U.S. Pat. No. 9,161,988 which is incorporated herein by reference in its entirety.

Other aspects of the present disclosure generally include methods of treating cancer comprising administering a therapeutically effective amount of at least one composition or compound disclosed to a subject in need thereof and/or contacting cells expressing Sortilin with at least one composition or compound herein disclosed. Other aspects include uses of the compositions, the peptide compounds, conjugate compounds described herein for treating cancer as well as in the manufacture of a medicament for treating a cancer.

In some aspects, the solubilizing agent is present in an amount from about 5% to about 15% by weight per total volume of the composition. In other aspects, the solubilizing agent is present in an amount from about 8% to about 12% by weight per total volume of the composition. In other aspects, the solubilizing agent is present in an amount from about 9% to about 11% by weight per total volume of the composition. In other aspects, the solubilizing agent is present in an amount of about 10% by weight per total volume of the composition.

In some aspects, the conjugate compound is present in an amount from about 0.1% to about 5% by w/w % based on the total weight of the composition. In some aspects, the conjugate compound is present in an amount from about 0.5% to about 2.5% by w/w % based on the total weight of the composition. In some aspects, the conjugate compound is present in an amount from about 0.5% to about 1.5% by w/w % based on the total weight of the composition. In some aspects, the conjugate compound is present in an amount from about 0.8% to about 1.2% by w/w % based on the total weight of the composition. In some aspects, the conjugate compound is present in an amount from about 0.9% to about 1.1% by w/w % based on the total weight of the composition.

In some aspects, the composition further comprises a solution suitable for injection that is present at about 1% to about 10%, by weight per total volume of the composition. In some aspects, the composition further comprises a solution suitable for injection that is present at about 2% to about 8%, by weight per total volume of the composition. In some aspects, the composition further comprises a solution suitable for injection that is present at about 3% to about 7%, by weight per total volume of the composition. In some aspects, the composition further comprises a solution suitable for injection that is present at about 4% to about 6%, by weight per total volume of the composition. In some aspects, the composition further comprises a solution suitable for injection that is present at about 5%, by weight per total volume of the composition.

In some aspects, the solubilizing agent is chosen from polysorbate (Tween™) polyethylene glycol (15)-hydroxystearate (Solutol™), dimethyl sulfoxide (DMSO), water-soluble organic solvents (polyethylene glycol 300, polyethylene glycol 400, ethanol, propylene glycol, glycerin, N-methyl-2-pyrrolidone, dimethylacetamide, and dimethylsulfoxide), non-ionic surfactants (Cremophor™ EL, Cremophor™ RH 40, Cremophor™ RH 60, d-α-tocopherol polyethylene glycol 1000 succinate, polysorbate 20, polysorbate 80, Solutol™ HS 15, sorbitan monooleate, poloxamer 407, Labrafil™ M-1944CS, Labrafil™ M-2125CS, Labrasol™, Gellucire™ 44/14, Softigen™ 767, and mono- and di-fatty acid esters of PEG 300, 400, or 1750), water-insoluble lipids (castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-α-tocopherol, oleic acid, medium-chain mono- and diglycerides), cyclodextrins (α-cyclodextrin, β-cyclodextrin, hydroxypropyl-β-cyclodextrin, and sulfobutylether-β-cyclodextrin), phospholipids (hydrogenated soy phosphatidylcholine, distearoylphosphatidylglycerol, L-α-dimyristoylphosphatidylcholine, L-α-dimyristoylphosphatidylglycerol).

In some aspects, the polysorbate is polysorbate 20, polysorbate 40, polysorbate 60 or polysorbate 80.

In some aspects, the composition of the present application further comprises a buffer chosen from an acetate buffer, borate buffer, citrate buffer, glycine buffe, HEPES buffer, phosphate buffer, Tris buffer, AES, Ammonia, AMP, AMPD, AMPSO, BES, Bicarbonate Bicine, BIS-Tris BIS-Tris-Propane Boric acid, Cacodylate, CAPS, CAPSO Carbonate, CHES, Citrate, DIPSO, Formate, Glycine Glycylglycine HEPES, HEPPS, EPPS HEPPSO Imidazole Malate, Maleate, MES, MOPS, MOPSO Phosphate, PIPES, POPSO, Phosphate, Pyridine Succinate, TAPS, TAPSO, Taurine, TEA, TES, Tricine, Tris and mixtures thereof.

In some aspects, the composition of the present application further comprises a dextrose solution (e.g. D5W), sodium lactate solution (Lactated Ringer's solution) saline, water, ethanol, acetic acid, formic acid, sodium hydroxide and mixtures thereof.

In some aspects, the composition is an aqueous solution having a pH of about 3 to about 5. In some aspects, the composition is an aqueous solution having a pH of about 3.5 to about 4.5. In some aspects, the composition is an aqueous solution having a pH of about 3.75 to about 4.25. In some aspects, the composition is an aqueous solution having a pH of about 3.8 to about 4.1.

In some aspects, the composition comprises a polysorbate, a dextrose solution, formic acid, sodium hydroxide and optionally water.

In some aspects, the polysorbate is present in an amount from about 5% to about 15% by weight per total volume of the composition. In other aspects, the polysorbate is present in an amount from about 8% to about 12% by weight per total volume of the composition. In other aspects, the polysorbate is present in an amount from about 9% to about 11% by weight per total volume of the composition. In other aspects, the polysorbate is present in an amount of about 10% by weight per total volume of the composition.

In some aspects, the dextrose solution has a concentration of from about 2% to about 8% and is present in an amount from about 2% to about 8% by weight per total volume of the composition. In other aspects, the dextrose solution has a concentration of from about 4% to about 6% and is present in an amount from about 4% to about 6% by weight per total volume of the composition. In some aspects, the dextrose solution has a concentration of about 5% and is present in an amount of about 5% by weight per total volume of the composition.

In some aspects, the formic acid is present in an amount from about 0.02% to about 0.06% by volume per total volume of the composition. In some aspects, the formic acid is present in an amount from about 0.03% to about 0.05% by volume per total volume of the composition. In some aspects, the formic acid is present in an amount of about 0.04% by volume per total volume of the composition.

In some aspects, the sodium hydroxide is in a solution having a concentration from about 0.05N to about 1.5N and is present in an amount such that the composition has a pH from about 4 to about 4.6. In some aspects, the sodium hydroxide is in a solution having a concentration from about 0.1N to about 1N. In some aspects, the sodium hydroxide is in a solution having a concentration of about 0.1N. In some aspects, the sodium hydroxide is in a solution having a concentration of about 1N. In some aspects, the sodium hydroxide solution is present in an amount such that the composition has a pH from about 4.1 to about 4.5. In some aspects, the sodium hydroxide solution is present in an amount such that the composition has a pH of about 4.3.

In some aspects, the composition comprises polysorbate 80 present in an amount of about 10% weight per total volume of the composition, a dextrose solution in a concentration of about 5% and present in an amount of about 5% by weight per total volume of the composition, formic acid present in an amount of about 0.04% by volume per total volume of the composition, sodium hydroxide in a solution having a concentration from about 0.1N to about 1N and present in an amount such that the composition has a pH from about 4.1 to about 4.5, and optionally water or a diluent.

In an aspect, there is provided a method of treating cancer or aggressive cancer comprising administering to a subject in need thereof a therapeutically effective amount of at least one composition or compound as defined herein.

In another aspect, there is provided a method of treating cancer or aggressive cancer in a subject having cancerous tissues or cells expressing Sortilin, comprising contacting said cancerous tissues or cells with at least one composition or compound as defined herein.

In another aspect, there is provided a method of treating cancer or aggressive cancer in a subject having cancerous tissues or cells expressing Sortilin, comprising contacting said cancerous tissues or cells with at least one composition or compound as defined herein.

In another aspect, there is provided a method of treating cancer or aggressive cancer in a subject having cancerous tissues or cells expressing Sortilin, comprising contacting said cancerous tissues or cells with at least one composition or compound as defined herein.

In another aspect, there is provided a method for minimizing, reducing or decreasing regrowth of tumors, comprising administering to a subject in need thereof a therapeutically effective amount of the composition as defined herein.

In some aspects, the composition is administered at a dose from about 1 to about 100 mg/kg/week. In some aspects, the composition is administered at a dose from about 2 to about 40 mg/kg/week. In other aspects, the composition is administered at a dose from about 5 to about 10 mg/kg/week. In other aspects, the composition is administered at a dose from about 5 to about 25 mg/kg/week. In other aspects, the composition is administered at a dose from about 10 to about 20 mg/kg/week. In other aspects, the composition is administered at a dose from about 10 to about 75 mg/kg/week. In other aspects, the composition is administered at a dose from about 35 to about 50 mg/kg/week.

In some aspects, the composition is administered at a dose from about 3 to about 300 mg/kg/three weeks. In other aspects, the composition is administered at a dose from about 6 to about 240 mg/kg/three weeks. In other aspects, the composition is administered at a dose from about 15 to about 30 mg/kg/three weeks. In other aspects, the composition is administered at a dose from about 15 to about 75 mg/kg/three weeks. In other aspects, the composition is administered at a dose from about 30 to about 60 mg/kg/three weeks. In other aspects, the composition is administered at a dose from about 30 to about 225 mg/kg/three weeks. In other aspects, the composition is administered at a dose from about 105 to about 150 mg/kg/three weeks.

In some aspects, the dose are exprimed in terms of active ingredient in the composition. For example, for exemplary formulations comprising a conjugate compound of docetaxel (TH1902), about 44% of the total weight of the conjugate compound corresponds to one molecule of docetaxel. In other words, 1eq. of docetaxel in weight would correspond to about 2.33 times the weight of the conjugate TH1902, i.e. 1 g of docetaxel=about 2.33 g of TH1902.

In some aspects, the composition is administered at a dose from about 3 to about 300 mg/mm²/week. In other aspects, the composition is administered at a dose from about 5 to about 210 mg/mm²/week. In other aspects, the composition is administered at a dose from about 75 to about 150 mg/mm²/week. In other aspects, the composition is administered at a dose from about 10 to about 300 mg/mm²/week. In other aspects, the composition is administered at a dose from about 30 to about 150 mg/mm²/week.

In some aspects, the composition is administered at a dose from about 10 to about 1000 mg/mm²/three weeks. In other aspects, the composition is administered at a dose from about 15 to about 500 mg/mm²/three weeks. In other aspects, the composition is administered at a dose from about 10 to about 250 mg/mm²/three weeks. In other aspects, the composition is administered at a dose from about 10 to about 500 mg/mm²/three weeks. In other aspects, the composition is administered at a dose from about 50 to about 450 mg/mm²/three weeks.

In some aspects, said composition prevents tumor growth or progression for a period of at least 10 days post-treatment. In some aspects, said composition prevents tumor growth or progression for a period of at least 20 days post-treatment. In other aspects, said composition prevents tumor growth or progression for a period of at least 30 days post-treatment. In other aspects, said composition prevents tumor growth or progression for a period of at least 40 days post-treatment.

In some aspects, said composition prevents tumor growth or progression for a period of about 10 to about 50 days post-treatment. In other aspects, said composition prevents tumor growth or progression for a period of about 10 to about 25 days post-treatment. In other aspects, said composition prevents tumor growth or progression for a period of about 10 to about 20 days post-treatment. In other aspects, said composition prevents tumor growth or progression for a period of about 10 to about 15 days post-treatment.

In some aspects, said composition is effective for reducing tumor size for a period of at least 10 days post-treatment. In other aspects, said composition is effective for reducing tumor size for a period of at least 20 days post-treatment. In other aspects, said composition is effective for reducing tumor size for a period of at least 30 days post-treatment. In other aspects, said composition is effective for reducing tumor size for a period of at least 40 days post-treatment.

In some aspects, said composition is effective for reducing tumor size for a period of about 10 to about 50 days post-treatment. In other aspects, said composition is effective for reducing tumor size for a period of about 10 to about 25 days post-treatment. In other aspects, said composition is effective for reducing tumor size for a period of about 10 to about 20 days post-treatment. In other aspects, said composition is effective for reducing tumor size for a period of about 10 to about 15 days post-treatment.

In some aspects, the subject is a mammal. In some aspects, the subject is an animal. In some aspects, the subject is a human.

In another aspect, there is provided a method of increasing stability and/or bioavailability of a therapeutic agent, comprising:

obtaining the composition or conjugate compound disclosed herein, wherein said composition or conjugate compound comprises said therapeutic agent, and
administering a therapeutically effective amount of said composition or conjugate compound to a subject in need thereof.

In another aspect, there is provided a method of increasing stability and/or bioavailability of a therapeutic agent, comprising:

conjugating said therapeutic agent with the peptide compound as defined herein to obtain a conjugate compound, and
administering a therapeutically effective amount of said conjugate compound to a subject in need thereof.

In another aspect, there is provided a method for increasing half life and/or stability of i) a peptide compound having at least 60% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII) or ii) a conjugate compound having the formula of A-(B)ₙ,

wherein
n is 1, 2, 3 or 4;
A is said peptide compound; and
B is at least one therapeutic agent, wherein B is connected to A at a free amine of a lysine residue of said peptide compound, optionally via a linker, or at an N-terminal position of said peptide compound, optionally via a linker,
comprising mixing the peptide compound or the conjugate compound with a solubilizing agent so as to increase the half life by at least 2 fold, at least 2.5 fold, at least 3 fold, at least 3.5 fold, at least 4 fold, at least 4.5 fold, at least 5 fold, at least 5.5 fold, or at least 6 fold.

In another aspect, there is provided a method for increasing half life and/or stability of a peptide having at least 60% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII), said method comprising conjugating the peptide compound with at least one molecule.

For example, the at least one molecule is at least one therapeutic agent. For example, the at least one therapeutic agent is an anticancer agent. For example, the anticancer agent is docetaxel.

For example, the at least one molecule is chosen from small molecules, peptides, proteins, oligonucleotides, diagnostic agents, imaging or radionuclide agents, large molecules such as monoclonal antibodies, drug delivery systems including nanoparticles, liposomes, nanotubes, graphene particles loaded with a therapeutic agent, imaging agent, gene, siRNA.

For example, the conjugated peptide has a half life increased by at least 1.5 fold, least 2 fold, at least 2.5 fold, at least 3 fold, at least 3.5 fold, at least 4 fold, at least 4.5 fold, at least 5 fold, at least 5.5 fold, at least 6 fold, at least 8 fold, at least 10 fold, at least 12 fold, at least 15 fold or at least 20 fold compared to the same peptide unconjugated peptide.

The conjugate compound herein disclosed may also provide greater tolerability compared to unconjugated therapeutic agents. For example, in the International application published as WO 2017/088058 and entitled PEPTIDE COMPOUNDS AND CONJUGATE COMPOUNDS FOR THE TREATMENT OF CANCER THROUGH RECEPTOR-MEDIATED CHEMOTHERAPY, filed Nov. 24, 2016 (herein incorporated by reference in its entirety), it has been shown in that peptide-drug conjugates are better tolerated compared to unconjugated therapeutic agents at an equivalent dose due to specific receptor targeting. In particular, in vivo studies showed that treatment with a conjugate compound had little effect on the body weight of tested mice thus demonstrating tolerability of the conjugate compound.

For example, there is provided herein a method of increasing tolerability of a therapeutic agent, comprising:

conjugating the therapeutic agent with a peptide compound herein disclosed to obtain a conjugate compound or an, and
administering a therapeutically effective amount of the conjugate compound to a subject in need thereof.

For example, there is provided herein a method of increasing tolerability of a therapeutic agent, comprising:

obtaining a conjugate compound herein disclosed, wherein the conjugate compound comprises the therapeutic agent, and
administering a therapeutically effective amount of the conjugate compound to a subject in need thereof.

For example, there is provided a use of a compound or composition herein disclosed, for increasing tolerability of a therapeutic agent.

In another aspect, there is provided a use of a compound or composition as defined herein for treating a cancer.

In another aspect, there is provided a use of a compound or composition as defined herein for targeting Sortilin receptor.

In another aspect, there is provided a use of a compound or composition as defined herein for treatment of cancer or aggressive cancer.

In another aspect, there is provided a use of a compound or composition as defined herein for treatment of cancer or aggressive cancer involving sortilin expression.

In another aspect, there is provided a use of a compound or composition as defined herein for treatment of cancer or aggressive cancer in cancerous tissues or cells expressing Sortilin.

In another aspect, there is provided a use of a compound or composition as defined herein for treatment of cancer or aggressive cancer in CD133 positive cells.

In another aspect, there is provided a use of a compound or composition as defined herein for increasing stability and/or bioavailability of at least one therapeutic agent.

In another aspect, there is provided a use of a compound or composition as defined herein for increasing stability and/or bioavailability of at least one peptide compound.

In another aspect, there is provided a use of a compound or composition as defined herein for minimizing, reducing or decreasing regrowth of tumors.

In another aspect, there is provided a use of a compound or composition as defined herein in the manufacture of a medicament for treating a cancer.

In another aspect, there is provided a use of a compound or composition as defined herein in the manufacture of a medicament for treatment of cancer or aggressive cancer.

In another aspect, there is provided a use of a compound or composition as defined herein in the manufacture of a medicament for treatment of cancer or aggressive cancer involving Sortilin expression.

In another aspect, there is provided a use of a compound or composition as defined herein in the manufacture of a medicament for treatment of cancer or aggressive cancer in cancerous tissues or cells expressing Sortilin.

In another aspect, there is provided a use of a compound or composition as defined herein in the manufacture of a medicament for treatment of cancer or aggressive cancer in CD133 positive cells.

In another aspect, there is provided a use of a compound or composition as defined herein in the manufacture of a medicament for minimizing, reducing or decreasing regrowth of tumors.

For example, the at least one therapeutic compound comprised in the conjugate compound and/or used in the manufacture of a medicament to treat a cancer is an anticancer agent. For example, the anticancer agent is chosen from docetaxel, cabazitaxel, aldoxorubicin maytansinoids, auristatins, calicheamicins, amatoxins, amanitin, and doxorubicin.

For example, the at least one therapeutic compound comprised in the conjugate compound and/or used in the manufacture of a medicament to treat a cancer is a phytochemical. For example, the phytochemical is curcumin.

For example, the phytochemical is chosen from curcumin, omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

In another aspect, there is provided a use of a compound or composition herein disclosed, for treating a cancer, in combination with a therapeutic agent such as cytotoxics, toxins and anticancer peptides; an immunodulator such anti-PD1 and anti-PDL1; an anticancer delivery system, an antiangiogenic agent; and/or radiotherapy.

In a further aspect, there is provided a use of a compound or composition herein disclosed, for targeting Sortilin receptor, in combination with a therapeutic agent such as cytotoxics, toxins and anticancer peptides; an immunodulator such anti-PD1 and anti-PDL1; an anticancer delivery system, an antiangiogenic agent; and/or radiotherapy.

Further provided is a method of preparing a composition of the present application, the method comprising: preparing a diluent solution comprising the solubilizing agent; adding the conjugate compound to the diluent solution in an amount sufficient to obtain a desired concentration; heating the solution to solubilize the conjugate compound; cooling the solution; adjusting pH of the solution to a pH from about 4 to about 4.6; optionally adding diluent to a final volume of the composition.

Further embodiments of the present disclosure will now be described with reference to the following Examples. It should be appreciated that these Examples are for the purposes of illustrating embodiments of the present disclosure, and do not limit the scope of the disclosure.

### EXAMPLES

### Example 1: Receptor-Mediated Therapy Using a Docetaxel-Peptide Conjugate for Sortilin-Positive Triple-Negative Breast Cancer

### Introduction

Taxanes are a widely used class of chemotherapeutic molecules which prevent microtubule depolymerization, which thus inhibits cell division. Examples of taxanes include paclitaxel and docetaxel. These taxanes are used to treat a variety of cancers, including breast cancer.

Sortilin is a molecule found at the cell surface of multiple tissues and in intracellular membrane locations. It functions as a receptor for several peptide molecules and plays a poorly understood role in targeting intracellular transport of membrane vesicles. Sortilin is overexpressed in many forms of cancer, including breast, ovarian, endometrial, lung, melanoma, colorectal and pancreatic cancers. Sortilin is found to be overexpressed in 79% of invasive ductal breast cancer and 59% of triple negative breast cancer (TNBC). TNBC accounts for 15-20% of breast cancers worldwide and remains the deadliest subgroup of breast cancer. BC is considered more aggressive than other breast cancers and more difficult to treat. Amongst women treated for TNBC, 42% will have rapid relapses with a peak 3 years after diagnosis. Currently, no targeted therapies are approved for the treatment of TNBC; thus, surgery, anthracycline-, taxane-based chemotherapy, and radiation therapy are the primary treatment options for patients with TNBC.

The TH19P01 peptide has been developed and specifically binds to the extracellular face of sortilin and which is internalized by the protein, enabling it to carry into the cell moieties which have been bound to the peptide. One molecule which has been investigated is TH1902, which contains two molecules of docetaxel ester-linked to the peptide, and which has shown considerable promise as a chemotherapeutic agent when tested with cell cultures and with human xenografts in nude mice.

### High Sortilin Expression in Human Breast Cancers

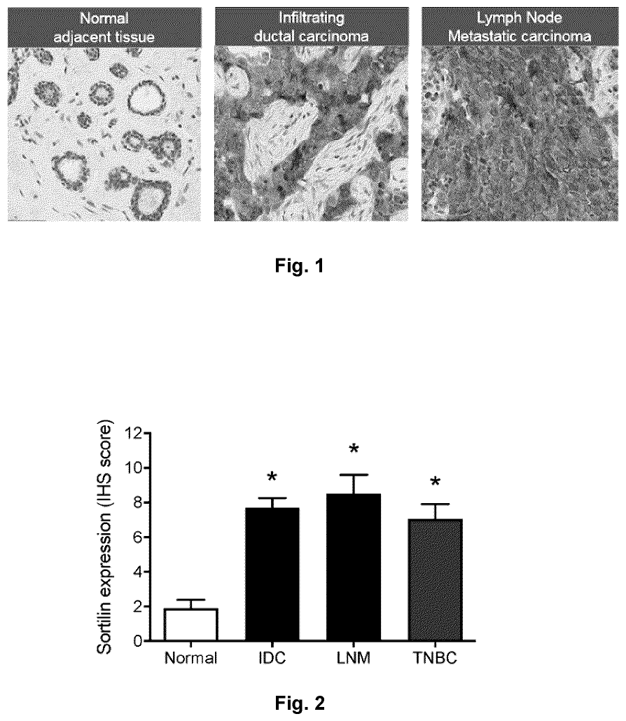

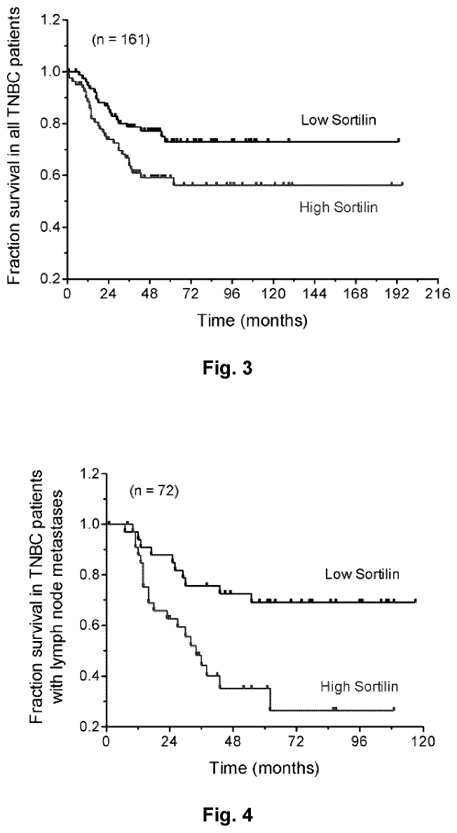

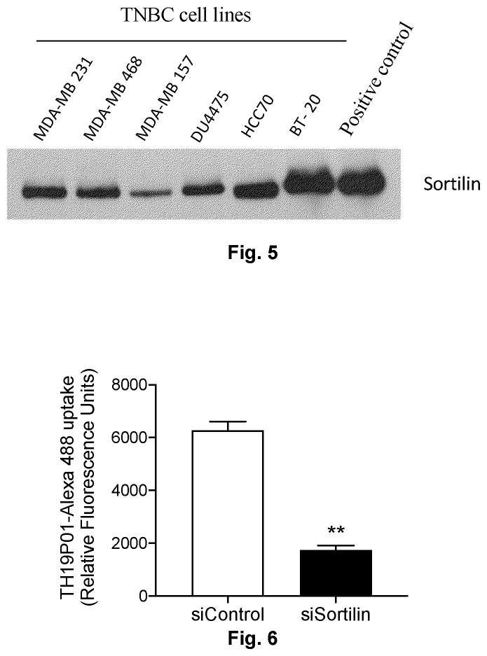

As shown in FIG. 1 and FIG. 2, high expression of sortilin in human breast cancers (infiltrating ductal carcinoma, lymph node metastatic carcinoma and triple-negative breast cancer) is shown using immunohistochemistry staining. The highest expression levels were detected in lymph node metastases. As shown in FIG. 3, Kaplan-Meier curves show that high sortilin gene expression is associated with a poor prognosis for TNBC patients in advanced stages 3 or 4 (n=161 cases). As shown in FIG. 4, Kaplan-Meier analysis of TNBC patients with lymph node metastases (n=72 cases) shows a drastic effect of high sortilin gene expression on patient survival. FIG. 5 is a western blot showing that Sortilin is highly expressed in different human TNBC cancer cell lines.

### Sortilin-Mediated Chemotherapy Internalization, Proliferation, Migration and Apoptosis

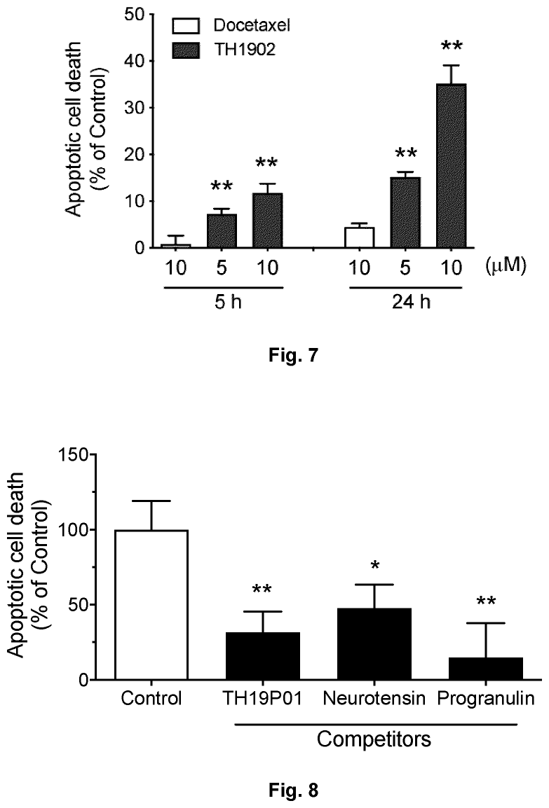

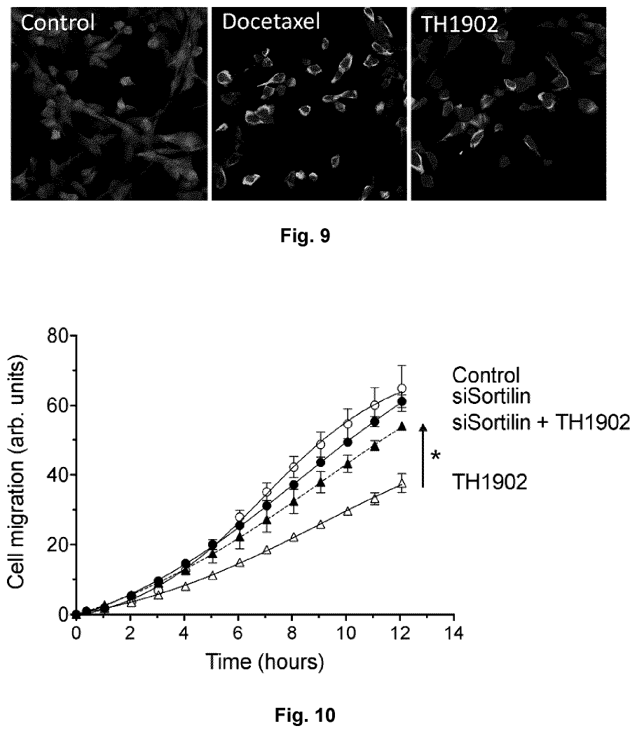

In vitro testing of the TH19P01 peptide and the TH1902 conjugate compound was conducted. As shown in FIG. 6, uptake of the peptide TH19P01 is inhibited upon sortilin siRNA. TH1902 was shown to have potent anti-proliferative activity in MDA-MB-231 breast cancer cells, with ah IC₅₀ value of 0.19±0.09 nM compared to 0.56±0.19 nM for docetaxel. In addition, apoptosis of MDA-MB-231 cells induced by TH1902 was found stronger than that of docetaxel (FIG. 7) and was found to be reversed by sortilin ligands TH19P01, neurotensin and progranulin (FIG. 8). TH1902 was also found to alter MDA-MB-231 microtubules polymerization, as shown by immunistaining of α-tubulin (FIG. 9). Cell migration was also found to be inhibited in a sortilin-dependent manner by TH1902 (FIG. 10).

### In Vivo Validation of Safety: Assessment of Neutropenia in TH1902 Treated Mice

One of the more common oncologic emergencies associated with the use of taxanes in chemotherapy is febrile neutropenia (defined as a cell count below 5×10⁸/L and expected to worsen).

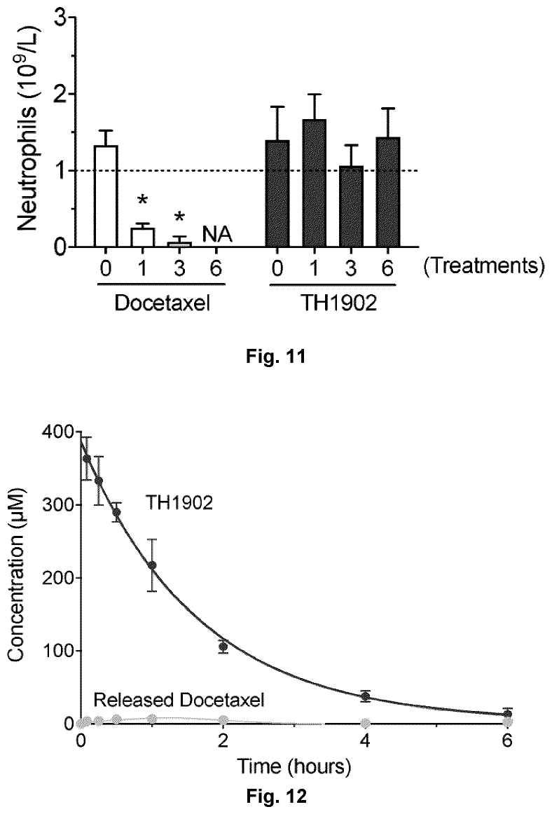

15 young adult, female, homozygous athymic mice (Crl:CD1-Foxn1^{nu}, 4-6 weeks of age) received 6 consecutive treatments of docetaxel or TH1902 (at equivalent dose of docetaxel (15 mg/kg/week)). As FIG. 11 shows, administration of docetaxel had induced a sharp drop in neutrophil levels by just four days after the first administration of docetaxel. This level continued to decrease as the administration of docetaxel continued. While the decreased neutrophil counts subsequent to administration of the docetaxel test article were statistically significant after both the first and third injections, there was no apparent change in the neutrophil counts for mice which received administration of the TH1902 test article at any measured time (up to 6 cycles; total dose 195 mg/kg) nor were there significant changes in the animals receiving vehicle alone (data not shown). These results indicated that TH1902 may be useful in preventing or reducing neutropenia, a side effect commonly observed with docetaxel treatment. In addition, no weight loss was observed in TH1902-treated mice (data not shown).

In addition, TH1902 and docetaxel mouse plasma levels were assessed in TH1902-treated mice. As shown in FIG. 12, a high plasma concentration of TH1902 was measured following IV bolus injection (50 mg/kg). A very low concentration of docetaxel released from TH1902 was measured in mouse plasma whereased about 15-20% of docetaxel concentration can be measured when administered as a free drug.

In Vivo Validation of Efficacy: Strong Inhibition of TNBC Tumor Growth in MDA-MB-231 s.c. Xenografts

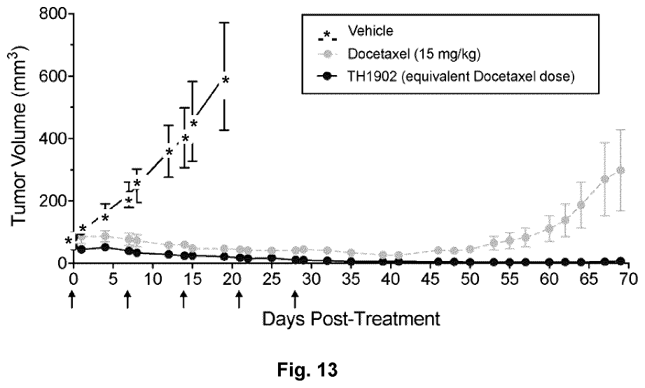

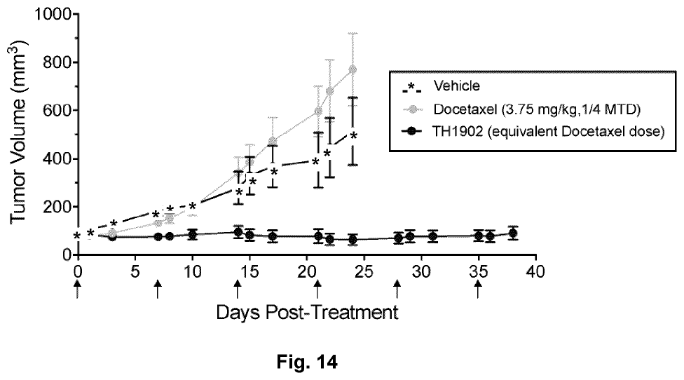

A first group of mice implanted with MDA-MB-231 s.c. xenografts received a high dose of docetaxel (15 mg/kg), TH1902 (at equivalent docetaxel dose) or vehicle (see FIG. 13). A second group of mice also implanted with MDA-MB-231 s.c. xenografts received a low dose of docetaxel (3.75 mg/kg, ¼ MTD), TH1902 (at equivalent docetaxel dose) or vehicle (see FIG. 14). In the first group (high dose), TH1902 was found to provide better and sustained efficacy in terms of tumor growth inhibition. In the second group (low dose), a significant improvement of efficacy was observed with TH1902 when administered at lower dose compared to docetaxel. In addition, a higher cumulative injected dose was seen for TH1902 (up to 2-fold) compared to docetaxel alone.

### Conclusion

TH1902 demonstrated improved tolerability (lower toxicity) and improved efficacy (stronger inhibition of TNBC tumor growth) compared to docetaxel alone (at equivalent dose). Other breast cancer types expressing Sortilin may potentially benefit from TH1902.

### Example 2: Increased In Vitro Stability of Formulated TH1902 in Mouse Plasma

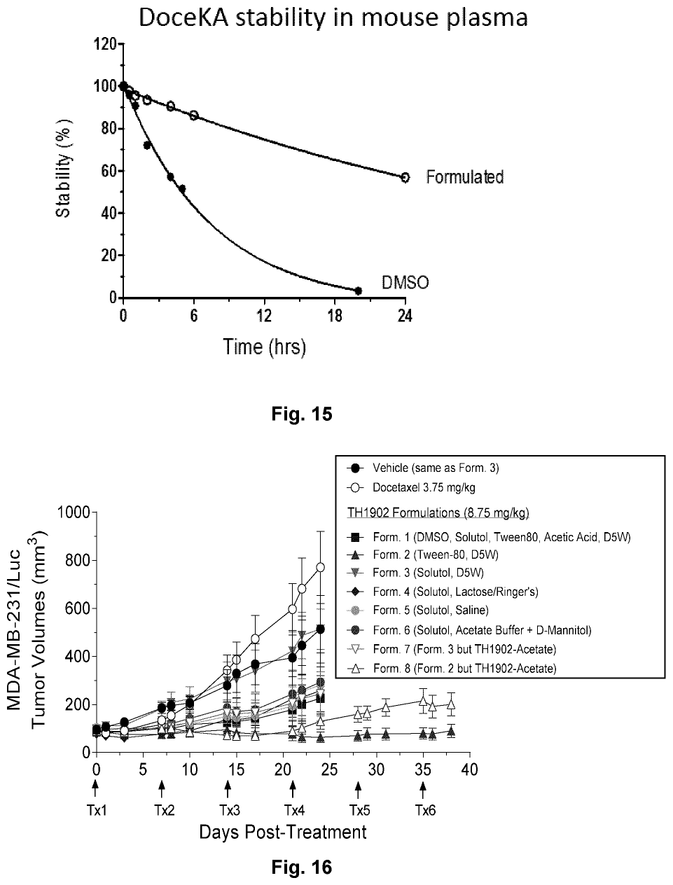

Additional testing was conducted to study the stability of formulated TH1902 in mouse plasma. TH1902 was either solubilized in DMO or formulated with solubilizing agents (Formulation: DMSO/Solutol™ HS15/Tween™-80/EtOH/Sol. Ac. Ac./D5W/water in the following V/V proportion 5/6/2.5/0.75/0.005/69/16.75). TH1902 was incubated in mouse plasma at 37° C. for the indicated times. Plasma proteins were precipitated by the addition of 4 volumes of ACN (87%) with formic acid (0.125%) followed by centrifugation (10,000 rpm×5 min). Supernatants were injected in UPLC/MS. Peak area corresponding to TH1902 was calculated and compared to time 0. Results were expressed in term stability (%) as a function of time, as shown in FIG. 15. As can be seen, TH1902 solubilized in DMSO had a half life of 5 hours whereas TH1902 formulated with solubilizing agents had a half life of 30 hours.

### Example 3: Improved Formulations of TH1902

### Example 3A: Formulations Comprising 8.75 mg/kg/Week TH1902

### Introduction

An important factor in the development of any pharmaceutical is the formulation of the administered product. This refers to the ensemble of chemicals that are present with the test article, which may influence solubility, stability, pH or another property that could affect the drug's bioavailability and activity.

### Objective

The objective was to compare tumor growth inhibition when infusing TH1902 (8.75 mg/kg/week) in different formulations against subcutaneous human tumor model MDA-MB-231/Luc (triple-negative breast cancer constitutively expressing luciferase) xenografts grown in nude mice in order to identify the optimal formulation.

### Methods

### Compound Characterization

TH1902 and docetaxel (supplied by Wonda Science Inc.) were used. Docetaxel (having a molecular weight of 808 g/mol) was formulated in EtOH/Tween™-80/D5W (1:1:78). Specifically, 2.5 mg of docetaxel was dissolved in 40 μl ethanol, then 40 μl Tween™-80 was added, followed by addition of 3120 μl D5W.TH1902 (having a molecular weight of 3704 g/mol) was formulated as described further below. Both treatments were administered intravenously, on a per week basis over 6 weeks. The treatment doses were 8.75 mg/kg for TH1902 (concentration of 1.35 mg/mL) and 3.75 mg/kg for docetaxel (concentration of 0.625 mg/mL).

8 different formulations comprising TH1902 were tested. For formulations 7 and 8 (described below), TH1902 was loaded onto an HPLC column and then rinsed with 0.25 M ammonium acetate, rinsed with 2% acetic acid and eluted with 50% acetonitrile, 2% acetic acid. The eluate (TH1902 with acetate counter-ion) was freeze-dried until use in Formulations 7 and 8. The various TH1902 formulations are detailed in Table 1 and Table 2 below.

TABLE 1
TH1902 Formulation components

| | | TH1902 |
|---|---|---|
| Formulation | Solution (% indicates v/v) | Counterion |
| Formulation 1 | 5% DMSO, 6% Solutol ™, | Formate |
| | 2.5% Tween ™ 80, | |
| | 0.75% EtOH, 0.5% acetic acid, | |
| | 69% D5W. | |
| Formulation 2 | 5% Tween ™ 80, 80% D5W | Formate |
| Formulation 3 | 10% Solutol ™, 25% D5W | Formate |
| | pH 2.95, 50% D5W | |
| Formulation 4 | 10% Solutol ™, 75% | Formate |
| | Lactose/Ringer’s pH 6.2 | |
| Formulation 5 | 10% Solutol ™, 75% saline | Formate |
| Formulation 6 | 10% Solutol ™, 7.5 mM acetate | Formate |
| | buffer, 3.75 mM D-mannitol | |
| Formulation 7 | 10% Solutol ™, 25% D5W | Acetate |
| | pH 2.95, 50% D5W | |
| Formulation 8 | 5% Tween ™ 80 (100% w/v), | Acetate |
| | 80% D5W | |

TABLE 2
Representative examples for formulations

| | Quantity | | | | |
|---|---|---|---|---|---|
| | TH1902 | | | | Final |
| Formulation | (mg) | Excipients | | Method | pH |
| Vehicle | — | 200 | μl Solutol ™ | Add 200 μl Solutol ™, 500 μl D5W | |
| | | 500 | μl D5W pH 4.4 | acidified to pH 4.4, 300 μl distilled | |
| | | 300 | μl distilled H₂O | water and 1 ml D5W | |
| | | 1 | ml D5W | (10/25/15/50). | |
| Formulation | 3.28 | 121 | μl DMSO | 1.5 mg/ml stock solution: Weigh | 4.08 |
| 1 | | 146 | μl Solutol ™ | 3.28 mg of TH1902, dissolve in | |
| | | 243 243 | μl | 121 μl of DMSO, add 146 μl of | |
| | | 25% | Tween 80 | Solutol ™, 243 μl of 25% | |
| | | 18 | μl EtOH | Tween ™ 80, 18 μl of EtOH, | |
| | | 12 | μl | 12 μl of 1% acetic acid, | |
| | | 1% | acetic acid | 462 μl of D5W, | |
| | | 462 | μl D5W | 213 μl of water and | |
| | | 213 | μl distilled H₂O | 1215 μl of D5W. | |
| | | 1.215 | ml D5W | | |
| Formulation | 3.2 | 119 | μl Tween ™ 80 | 1.5 mg/ml stock solution: Weigh | 3.90 |
| 2 | | 711 | μl D5W pH 2.95 | 3.2 mg of TH1902, dissolve in | |
| | | 356 | μl distilled H₂O | 119 μl of Tween ™ 80, add | |
| | | 1.185 | ml D5W | 711 μl of D5W pH 2.95, | |
| | | | | 356 μl of water | |
| | | | | and 1185 μl of D5W. | |
| Formulation | 3.28 | 237 | μl Solutol ™ | 1.5 mg/ml stock solution: Weigh | 4.40 |
| 3 | | 593 | μl D5W H 2.95 | 3.28 mg of TH1902, dissolve in | |
| | | 356 | μl distilled H₂O | 237 μl of Solutol ™, add 593 μl | |
| | | 1.185 | ml D5W | of D5W pH 2.95, 356 μl of water | |
| | | | | and 1185 μl of D5W. | |
| Formulation | 3.04 | 225 | μl Solutol ™ | 7.5 mg/ml stock solution: Weigh | 4.98 |
| 4 | | 563 | μl Lactose/ | 3.04 mg of TH1902, dissolve into | |
| | | | Ringer’s | 225 μl of Solutol ™, add 563 μl of | |
| | | | solution | Lactose/Ringer’s pH 6.2, 338 μl | |
| | | | pH 6.2 | of water and 1126 μl of | |
| | | 338 | μl distilled H₂O | Lactose/Ringer’s pH 6.2. | |
| | | 1.126 | ml Lactose/ | | |
| | | | Ringer’s | | |
| | | | solution pH 6.2 | | |
| Formulation | 2.72 | 201 | μl Solutol ™ | 1.5 mg/ml stock solution: Weigh | 4.29 |
| 5 | | 504 | μl saline H 2.7 | 2.72 mg of TH1902, dissolve in | |
| | | 302 | μl distilled H₂O | 201 μl of Solutol ™, add 504 μl | |
| | | 1.007 | ml D5W | of saline pH 2.7, 302 μl of water | |
| | | | saline | and 1007 μl of saline. | |
| Formulation | 2.96 | 219 | μl Solutol ™ | 1.5 mg/ml stock solution: Weigh | 4.29 |
| 6 | | 548 | μl 10 mM | 2.96 mg of TH1902, dissolve in | |
| | | | acetate buffer | 219 μl of Solutol ™, add 548 μl | |
| | | | pH 3.8 with | of 10 mM acetate buffer pH3.8 | |
| | | 10 | mM D-mannitol | containing 5 mM D-mannitol, | |
| | | 329 | μl distilled H₂O | 329 μl of water and 1096 μl | |
| | | 1.096 | ml | of 10 mM acetate buffer | |
| | | 10 | mM acetate | pH 3.8 containing | |
| | | | buffer | 5 mM D-mannitol. | |
| | | | pH 3.8 with | | |
| | | 10 | mM D-mannitol | | |
| Formulation | 2.64 | 196 | μl Solutol ™ | 1.5 mg/ml stock solution: Weigh | 4.53 |
| 7 | (acetate | 489 | μl D5W | 2.64 mg of TH1902-acetate, | |
| | counter- | | pH 2.95 f | dissolve in 196 μl of Solutol ™, | |
| | ion) | 293 | μl distilled H₂O | add 489 μl of D5W acidified to | |
| | | 978 | μl D5W | 2.95, 293 μl of water and | |
| | | | | 978 μl of D5W. | |
| Formulation | 3.04 | 113 | μl Tween ™ 80 | 1.5 mg/ml stock solution: Weigh | 3.90 |
| 8 | | 678 | μl D5W pH 2.95 | 3.04 mg of TH1902-acetate, | |
| | | 338 | μl distilled H₂O | dissolve in 113 μl of Tween ™ 80, | |
| | | 1.126 | ml D5W | add 676 μl of D5W pH 2.95, | |
| | | | | 338 μl of water and | |
| | | | | 1126 μl of D5W. | |

### Tumor Cell Preparation

The cells used were MDA-MB-231/Luc epithelial breast adenocarcinoma cells (Cell Biolabs Inc. #AKR-231). These are derived from a triple-negative breast cancer (TNBC) and they stably express firefly luciferase. The MDA-MB-231/Luc cell line was grown as adherent monolayers at 37° C. in a humidified atmosphere (5% CO₂, 95% O₂). The culture media was DMEM medium (Wisent, #319-005-CL) supplemented with 1× Non-Essential Amino Acids (NEAA) 100× solution (Hyclone™, #30238.01) and 10% Fetal Bovine Serum (FBS) (Hyclone™, #SH30396.03). For experimental use, cells were detached from the culture flask by a 5-10-minute treatment with trypsin (Wisent, #325-042-CL), then were 10-fold diluted and neutralized by addition of complete culture media. Cell counts and cell viability were assessed with a BioRad TC20™ automated cell counter. For subcutaneous implantation in mice, MDA-MB-231/Luc tumor cells were resuspended in an appropriate volume of HBSS (Sigma #H6648) implantation media in order to inject 5×10⁶ cells in 150 μl (3.33×107 cells/ml).

### Animals

60 young adult, female, homozygous nude mice (Crl:CD1-Foxn1^{nu}), obtained from Charles River Canada Inc. (St-Constant, Quebec) were used in this study. Healthy mice were selected based on normal veterinary examination. Mice of comparable age (28-42 days) and body weight were retained for this study.

The mice were implanted subcutaneously with MDA-MB-231/Luc cells as described above. Tumor growth was monitored, and mice were randomly assigned to groups the vehicle group, docetaxel group or one of the 8 TH1902 formulations group (6 mice per group) for further treatment when tumor volumes reached 40-140 mm³ or after 2-3 days of significant increase. Experimental procedures

Mice were first anesthetized with isoflurane and oxygen. Two-dimensional measurements were taken with electronic calipers during the study and tumor volume was calculated using the following formula: tumor volume (mm³)=0.52*a*b², where 0.52 is a constant to calculate the volume of an ellipsoid (π/6), where “a” is the longest diameter and “b” is the shortest diameter[3].

Treatments began when tumor volume reached 40-140 mm³ or after 2-3 days of significant increase. All treatments were continued through 24 days, encompassing 4 treatments; animals receiving Formulations 2 and 8 continued for 2 more treatments to extend exposure to TH1902 through Day 38.

All mice were observed daily for changes in appearance and behavior, and events recorded when appropriate. Body weights were measured three times a week; they were recorded with a precision of 10 mg.

All animals in the Vehicle group were sacrificed once any of their tumors reached the size of 1000 mm³. The docetaxel and the 35 mg/kg TH1902 groups (equimolar of docetaxel to allow comparison) were maintained for prolonged treatment and observation until Day 69 when they were sacrificed.

The data was analyzed either by one-way ANOVA followed by Dunnett's test using animals treated with test Formulations and docetaxel vs vehicle, or (for comparing the growth curves for tumor volume or for animal bioluminescence) via non-linear regression fitting to a Gompertz growth curve. The analysis was done using GraphPad Prism software. Statistical significance was assumed for p<0.05.

### Results and Discussions

### Tumor Volumes

All tumors were measured with calipers three times weekly. This continued for a total of 12 measurements within 24 days. Afterwards, the animals were euthanized aside from those treated with Formulations 2 and 8, which had exhibited the greatest reduction in tumor volumes. These animals were maintained and monitored for another two weeks. The measured tumor volumes can be seen in FIG. 16. As can be seen, the black and white circles shown represent animals treated with vehicle and with free docetaxel at ¼ of its maximum tolerated dose (MTD), respectively; the docetaxel-treated mice have tumor burdens that appear to grow slightly faster than those in the control animals treated with vehicle alone. Of the 8 formulations, Formulation 3 appears to have no effect on tumor inhibition. In contrast, Formulations 2 and 8 show strong inhibition of tumor growth. The other 5 formulations exhibit intermediate levels of tumor inhibition and are comparable. Formulations 2 and 8 which show the strongest effects comprise a slightly acidic solution containing Tween-80 and dextrose; Formulation 2 contains TH1902 with a formate counterion while Formulation 8 contains TH1902 with an acetate counterion. The animals treated with Formulations 2 and 8 were monitored for another two weeks, during which time the tumors treated with Formulation 8 showed slow tumor growth while the tumor stasis observed with Formulation 2 remained unchanged. Formulations 2 and 8 showed statistically significant inhibition of tumor growth compared to vehicle-treated tumors, with a near-stasis at Day 24 observed for mice treated with Formulation 8 (p=0.04) and a slight decrease in tumor volume following treatment with Formulation 2 (p=0.01).

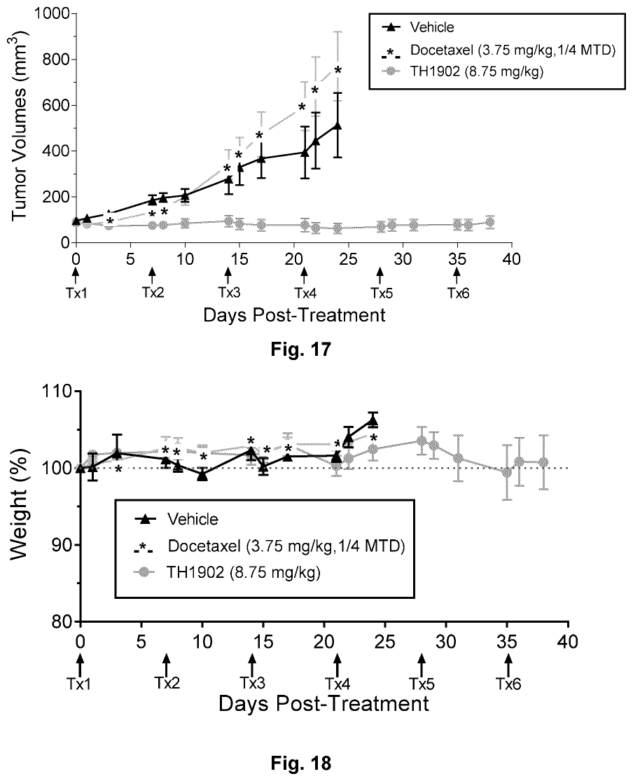

The strong inhibition in tumor volume growth induced by dilute TH1902 (8.75 mg/kg/week) in Formulation 2 is reproduced in FIG. 17 and the body weight of mice treated with Formulation 2 is shown in FIG. 18. As can be seen, treatment of mice with Formulation 2 had no impact on their body weights. Similarly, no impact on body weight was seen in other Formulations tested (data not shown).

### Example 3B: Formulations Comprising 17.5 mg/kg/Week TH1902

Additional formulations were tested as described in Table 3 below. The concentration (Conc.) of TH1902 in various formulations was assessed, as a measure of solubility of TH1902 in the formulation.

TABLE 3
Additional TH1902 formulations

| | Proportions | Cone, |
|---|---|---|
| Formulations | (V/V or W/V) | (mg/ml) |
| D5W /Tween ™ 80 | 80/20 | 10 |
| DMSO/Solutol ™ HS15/ | 5/6/2.5/0.75/0.005/ | 4-8 |
| Tween ™ 80/EtOH/ | 69/16.75 | |
| Sol. Ac. Ac./D5W/water | | |
| D5W/Tween ™ 80/water | 80/5/15 | 3-6 |
| D5W/Solutol ™ HS15/water | 75/10/15 | 3-6 |
| Ringer’s Lactated Solution/ | 75/10/15 | 3-6 |
| Solutol ™ HS15/water | | |
| Saline/Solutol ™ HS15/water | 75/10/15 | 3-6 |
| Acetate Bf 10 mM with | 75/10/15 | 3-6 |
| 5% D-mannitol/ | | |
| Solutol ™ HS15/water | | |
| D5W/Cremophor ™ EL/EtOH | 85/10/5 | 3-6 |
| Saline/Solutol ™ HS15 | 70/30 | 3-6 |
| D5W/Cremophor ™ ELP/EtOH | 85/10/5 | 1-2 |
| D5W/Labrafi ™\| | 80/20 | — |
| Beta-Cyclodextrin/water | 10/90 | — |
| Methyl-Beta-Cyclodextrin/water | 10/90 | 3-6 |
| 2 Hydroxypropyl-gamma- | 10/90 | — |
| Cyclodextrin/water | | |
| 2 Hydroxypropyl-Beta- | 10/90 | 3-6 |
| Cyclodextrin/water | | |
| Alpha-Cyclodextrin/water | 10/90 | — |
| Sulfobutylether-Beta- | 10/90 | — |
| Cyclodextrin/water | | |

“—” is indicative the conjugate compound TH1902 was not soluble in the particular formulation.

The preferred formulations, displaying a solubility of 3 mg/mL or greater, were subject to xenograft tumor volume testing. More specifically, Formulations 1 to 7 described Example 3A, were tested, with TH1902 doses of 17.5 mg/kg/week, in accordance with the method described in Example 3A.

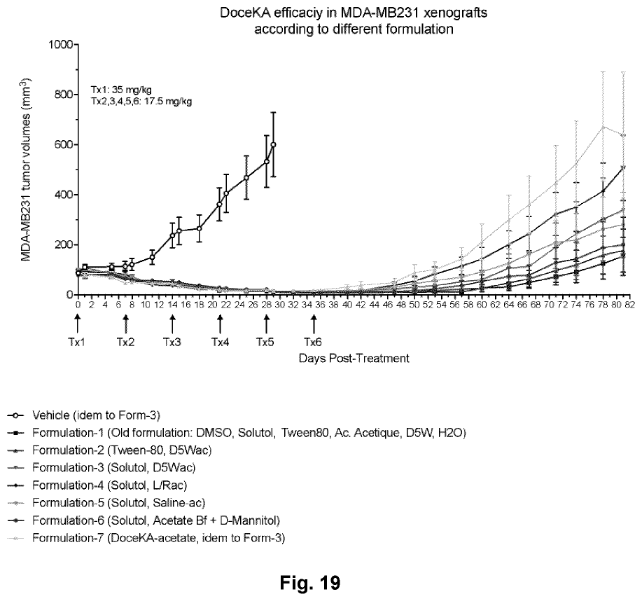

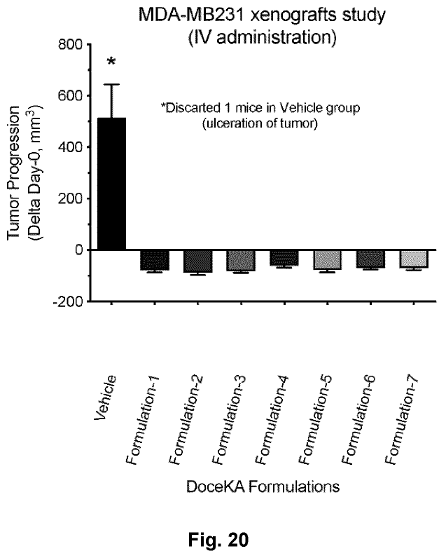

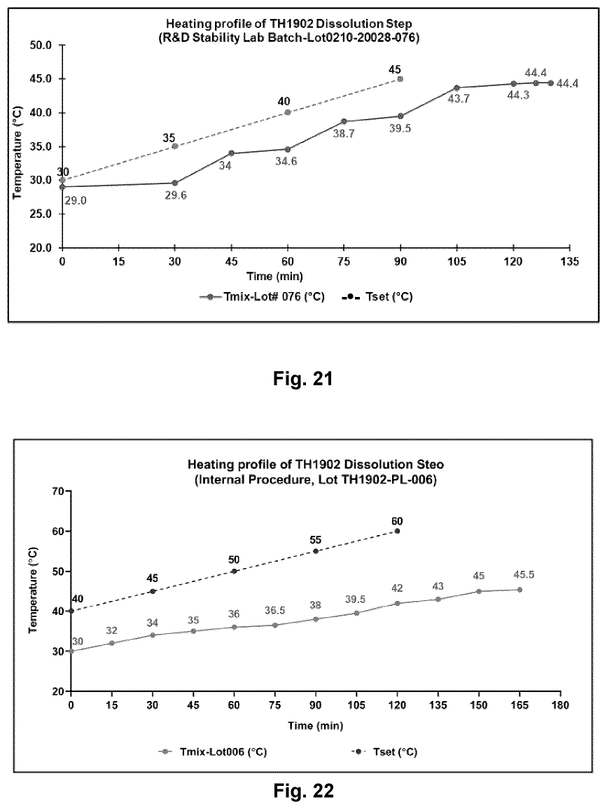

As can be seen in FIG. 19, after 6 treatments, all Formulations 1 to 7 were found to inhibit tumor volume, compared to the control. FIG. 20 similarly shows tumor progression in various TH1902 Formulations. From these results, it can be seen that the formulations of the present application are useful for minimizing, reducing or decreasing regrowth of tumors.

Table 4 below summarizes the study results of Example 3A and 3B, based on the various Formulations.

TABLE 4
Study results of low and high dose TH1902

| | 17.5 mg/kg/week of TH1902 | | | 8.75 mg/kg/week of TH1902 | |
|---|---|---|---|---|---|
| | ΔT/ΔC | Tumor | | ΔT/ΔC | Tumor |
| | (%) (at | Regression | Complete | (%) (at | Regression |
| | endpoint, | (at endpoint, | response | endpoint, | (at endpoint, |
| Formulation | Day-29) | Day-29) | (at Day-64) | Day-24) | Day-24) |
| 1 | −91% | 8/8 (100%) | 3/8 (38%) | 29% | 3/6 (50%) |
| 2 | −90% | 8/8 (100%) | 3/8 (38%) | −5% | 4/6 (66%) |
| 3 | −86% | 6/6 (100%) | 3/6 (50%) | 87% | 0/6 (0%) |
| 4 | −81% | 7/7 (100%) | 1/7 (15%) | 38% | 2/6 (33%) |
| 5 | −83% | 8/8 (100%) | 2/8 (25%) | 39% | 1/6 (17%) |
| 6 | −88% | 6/6 (100%) | 1/6 (17%) | 43% | 2/6 (33%) |
| 7 | −81% | 8/8 (100%) | 2/8 (25%) | 31% | 2/6 (33%) |
| 8 | NA | NA | NA | 9% | 2/6 (33%) |
| Docetaxel | NA | NA | NA | 143% | 0/6 (0%) |

### Example 4: Formulation in Clinical Setting

By extrapolating from the previous study results to a clinical setting, a proposed formulation is provided wherein 200 mg of the conjugate compound (e.g. TH1902) is dissolved in 20 mL Tween™ 80 (10%) in D5W (pH 3). The mixture is heated (10 min, 60° C.) then transferred into a D5W infusion bag (pH 5). The concentration of the conjugate peptide is about 0.5-2 mg/mL and that of Tween™ is about 0.5-2%.

### Example 5A: Formulation Composition for TH1902—Injection Concentrate

Table 5 shows components and concentration of an injection concentrate composition of TH1902 of 10 mg/mL.

TABLE 5
Composition of TH1902 Injection Concentrate 10 mg/mL

| | | | % w/w (based on density |
|---|---|---|---|
| Component | Concentration | | value 1.028 g/mL) |
| TH1902 API | 10 | mg/mL | 0.973 |
| Polysorbate 80 | 10% | w/v | 9.73 |
| Dextrose | 5% | w/v | 4.86 |
| Formic acid | 0.04% | v/v | 0.047 |

| | | |
|---|---|---|
| 1N NaOH | adjust to final | adjust to final |
| | pH 4.3 ± 0.2 | pH 4.3 ± 0.2 |
| Water for | Q.S. | Q.S. |
| Injection | | |

Density of the formulation is 1.029 g/mL at 25° C. (reference PPS Notebook: LNB-20-028 p045).
Density of formic acid = 1.22 g/mL. Density of Formic acid 99% = 1.213 g/mL.

### Dissolution of API

A 1.5-litre scale-up batch of TH9102 Injection 10 mg/mL was successfully prepared executing the approved protocol CSR0210-001.00. The compounding method specified in the protocol was based on the procedure developed previously for several small-scale lab batches in FRD. This procedure relied on careful heating of the compounding mixture between 40-45° C. for achieving the complete dissolution of the API and not exceeding this temperature range to avoid untoward gelation or aggregation of formulation. Based on the test results of the drug product quality attributes, the compounding procedure is concluded to be reproducible.

FIG. 21 shows heating profile during dissolution of TH1902 API of R&D Stability lab batch, according to Example 5A.

Initial activities on the formulation development of TH1902 10 mg/mL Injection Concentrate drug product focused on optimizing a solution that can effectively dissolve TH1902 API. Based on the outcome of studies conducted on excipient screening, additional excipient amounts (0 to 5% of dextrose) and acid selection (HCl, citric acid, formic acid), the solution was finalized as an aqueous mixture of polysorbate-80 (10% w/v), dextrose (5% w/v) and formic acid (0.04% v/v). The next stage of formulation activities focused on developing a compounding procedure for the effective dissolution of TH1902 at 10 mg/mL concentration in the finalized placebo. This compounding procedure was demonstrated on multiple small scale lab batches using several lots of API lots. The pH value of the formulation was also optimized in these studies. Final target pH value of TH1902 10 mg/mL was set to 4.3±0.2. Further, compounding procedure was found to be reproducible for two scale-up batches of 2.5 L and of 1.0 L (R&D stability lab batch) size at the set target pH value.

### Example 5B: Formulation Composition for TH1902—Injection Concentrate

An alternate composition of an injection concentrate of TH1902 of 10 mg/mL is provided. Table 6 shows components and concentration.

TABLE 6
Composition of TH1902
Injection Concentrate 10 mg/mL

| Component | Concentration | |
|---|---|---|
| TH1902 API | 10 | mg/mL |
| Polysorbate 80 | 10% | w/v |
| 5% Dextrose solution | 5% | w/v |
| for injection, | | |
| pH5 (D5W) | | |
| Formic acid | 0.04% | v/v |

| | |
|---|---|
| 0.1N NaOH | adjust to final |
| | pH 4.3 ± 0.2 |
| Diluent (D5W, | Q.S. |
| polysorbate 80 | |
| 10%, formic acid | |
| 0.04%, pH 3) | |

### Dissolution of API

Briefly, stock solution of formulated TH1902 was prepared as sterile aliquots of 10 mg/mL frozen liquid solutions (see below). On the day of animal dosing, frozen aliquots were thawed 30 minutes at room temperature then diluted with sterile 5% dextrose injection, USP (D5W) to the desired concentration for injection (i.e. typically 5.4 or 1.35 mg/mL).

FIG. 22 shows heating profile during dissolution of TH1902 API with an internal procedure, according to Example 5B.

TH1902 frozen stock solution were prepared as follow:
1. Make the TH1902 diluent solution (Diluent) the day before the solubilization. Diluent: 10% Tween™ 80 in D5W USP (w/v) with 0.04% of formic acid (v/v), ˜pH 2.9. Leave solution at room temperature until TH1902 solubilization (next day).
2. Weigh 60 mg of TH1902 API into a 14 ml glass vial with screw caps. Adjust TH1902 weights to reflect TH1902 purity according to the certificate of analysis.
3. Add 90% of diluent solution needed to prepare the TH1902 10 mg/ml stock solution.
4. Swirl the content of the bottle to form opaque mixture and place a stir bar.
5. Place bottle in the heating apparatus (glass water bath and mount for holding vial with thermometer and stir bar on a digital heating plate). Begin mixing the content of the bottle.
6. Gradually increase the temperature of water bath in increments of 5° C. every 30 minutes until mixture turns clear (temperature ramping up to 45° C. over a period of time of about 165 to 180 minutes). Record water bath temperature every 15 minutes.
7. Leave at room temperature for 30 minutes with gentle swirling (cool down). Measure pH. Adjust pH to 4.3±0.2 using dilute NaOH (0.1 N or less).
8. Transfer the formulation to a graduated glass cylinder and bring to final calculated volume with diluent solution.
9. Transfer back into the 14 ml glass vial and mix slowly for 5 minutes at room temperature. Measure pH. pH should be 4.3±0.2.
10. Filter sterilize with a PES 0.22 μm membrane into a new and sterile 14 ml glass vial. Check the pH again. Perform UPLC analysis against a TH1902 standard curve for quantification and purity assessment.
11. Freeze aliquots of TH1902 stock solution at −80° C. (0.5 mL per aliquot in 4 mL glass vials).

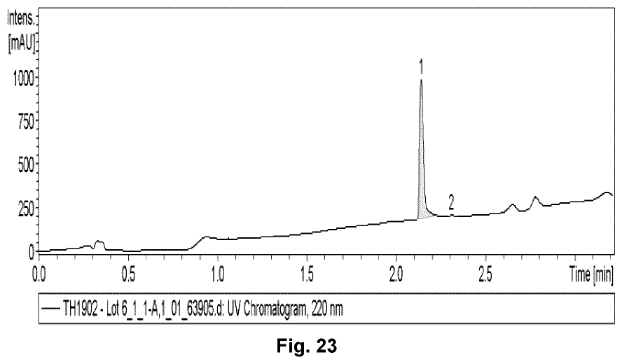

The representative UPLC analysis of the stock solution of TH1902 at 10 mg/ml following dissolution with the internal procedure is shown in FIG. 23.

Advantageously, it was shown that the formulations of Examples 5A-B provide for better appearance of the injectable product. Importantly, the heating profiles show that less heating is required to obtain these formulations. As such, one may expect for a greater stability of the formulations and better reproducibility of the methods for obtaining the same.

In Vivo Results Obtained with the Method of TH1902 API Dissolution (Internal Procedure)—Example 5B

### Endometrial Cancer Xenograft Model (AN3-CA) In Vivo Results

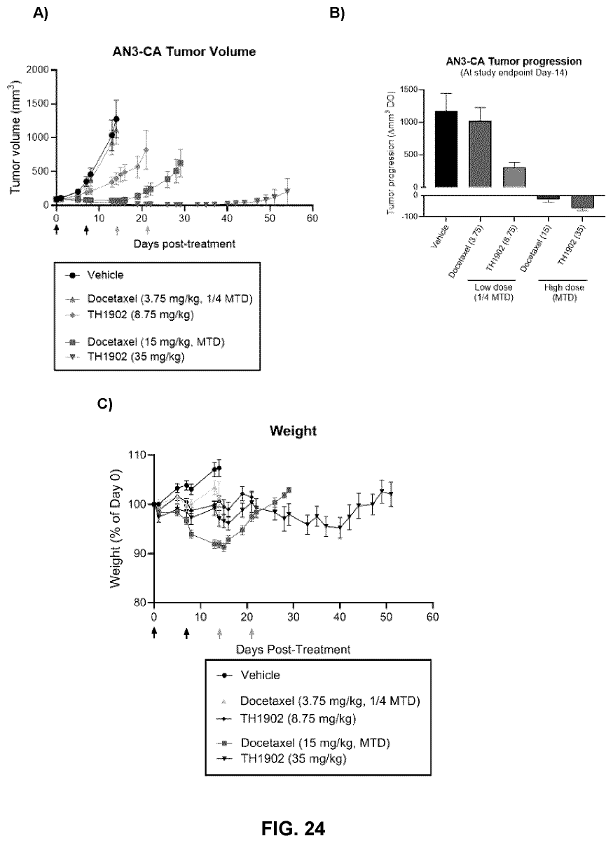

Three human sortilin-positive gynecological cancer cell lines (ovary: ES-2 and SKOV-3/Luc, endometrial: AN3-CA) were used for to assess the chemotherapeutic activity of TH1902 in vivo using xenograft models in immunodeficient mice. In the human AN3-CA endometrial xenograft tumor model (FIG. 24), unlike docetaxel, 3.75 mg/kg TH1902 (a concentration with the same amount of contained docetaxel) inhibited tumor growth. At an equivalent dose of docetaxel 15 mg/kg/week, both docetaxel and TH1902 administration caused complete arrest tumor growth arrest. TH1902 formulations appear to be more effective than docetaxel by inducing a strong regression of AN3-CA tumor volume. In fact, 5/6 mice treated with the highest dose of TH1902 had prolong tumor regression whereas slow tumor relapse was observed in only one mouse 30-days after the last treatment. Body weights of the mice administered with docetaxel, TH1902 or vehicle were monitored as a gross indicator of morbidity. As FIG. 24 demonstrates, the mice bearing the AN3-CA tumors exhibited a slight weight gain except for animals administered either of the test articles. At the MTD for docetaxel, on the other hand, there was greater evidence of weight loss associated with docetaxel, though the animal weights remained within the pre-set 20% weight loss endpoint limit. Animals administered TH1902 containing the equivalent quantity of docetaxel retained a fairly constant weight throughout the experiment.

The effect of TH1902 or docetaxel on the endometrial AN3-CA xenograft tumor model is shown on FIG. 24. For the purpose of FIG. 24A, mice bearing AN3-CA xenografts were repeatedly injected intravenously (arrows indicate injection days) with vehicle or with docetaxel (3.75 mg/kg/week, 15 mg/kg/week) or equivalent TH1902 doses (8.75 mg/kg/week and 35 mg/kg/week). After 2 cycles, both Docetaxel and TH1902 highest doses were reduced by half (7.5 mg/kg/week and 17.5 mg/kg/week, respectively) as indicated by grey arrows. In FIG. 24B effects of TH1902 and docetaxel on tumor progression at day-14 are presented by subtracting the initial tumor volumes at day-0 to the tumor volumes measured at day 14. For FIG. 24C, mouse body weights were monitored during the studies and were within the acceptable range and within the −20% endpoint limit during all the study. All data symbols shown represent means±standard error of the mean (SEM).

### Colorectal Cancer Xenograft Model (HT-29) In Vivo Results

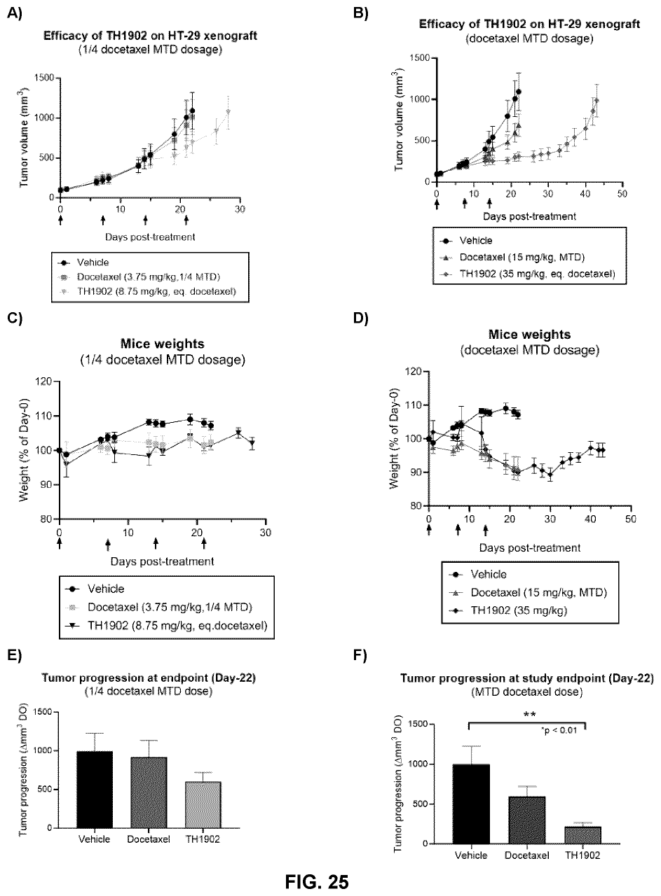

Mice bearing HT29 colorectal xenograft tumors were treated with low- and high-doses of docetaxel or TH1902 (FIG. 25). At the equivalent highest dose, TH1902 formulation caused a strong inhibition of HT29 tumor growth. Thus, TH1902 in the current formulation appears to be more effective than docetaxel by inducing a prolonged HT29 tumor progression.

FIG. 25 shows the effect of TH1902 or docetaxel on the colorectal HT29 xenograft tumor model. Mice bearing HT29 tumor xenografts were repeatedly injected intravenously (arrows indicate injection days) with vehicle or with vehicle containing low-doses (FIG. 25A) or high-doses of docetaxel or TH1902. Body weights of mice treated with equivalent low-doses (Fig. C) or high doses (Fig. D) of docetaxel or TH1902 were monitored during the studies and were within the acceptable range and within the −20% endpoint limit during all the study. All data symbols shown represent means±standard error of the mean (SEM).

### Pancreatic Cancer Xenograft Model (PANC-1) In Vivo Results

A sortilin-expressing pancreatic cancer cell line (PANC-1) was grown in vivo as subcutaneous tumor xenografts in immunocompromised nude mice. These animals were then used to assess the chemotherapeutic activity of vehicle, docetaxel and the peptide-drug conjugate TH1902 at two different doses. The growth of tumors in all five groups of mice was followed for 21 days; during this time there was steady growth of tumors in the animals treated with vehicle alone, and the tumors in mice treated with 3.75 mg/kg docetaxel appeared to be very similar in size to those treated with vehicle (FIG. 6A). Decreased tumor growth was associated with exposure to both low dose TH1902 (8.75 mg/kg) and to high dose docetaxel (15 mg/kg, its MTD). Exposure to high dose TH1902 (35 mg/kg) actually produced regression in tumor size.

The two groups receiving low doses of the test articles were continued to receive weekly test article administration and tumor volume measurements for another 1 to 4 weeks in order to examine the long-term consequences of low dose treatments (FIG. 6B). Tumor growth continued until the animals had to be euthanized due to tumor size, but it is evident that low dose administration of TH1902 inhibited tumor growth.

The growth curves of these tumors indicate that the low dosage administration of both docetaxel and TH1902 has less effect on tumor volumes than have higher doses. In addition, TH1902 appears to be more effective than docetaxel in halting tumor growth and actually produced tumor regression when administered at the higher dose.

The tumor volumes at day 21 were compared for the five groups of mice by one-way ANOVA followed by Dunnett's multiple comparison test, comparing tumor sizes in each of the four test groups against the tumor sizes in vehicle-treated mice. As FIG. 6C demonstrates, tumor sizes in mice bearing PANC-1 tumors were indistinguishable whether they had been administered vehicle or low dose docetaxel. Tumor sizes in mice receiving low dose TH1902 or high doses of either docetaxel or TH1902, on the other hand, showed significant decreases in tumor size, and even remission in the case of the high dose TH1902. These data confirm that TH1902 is superior to docetaxel in the treatment of these pancreatic xenograft tumors.

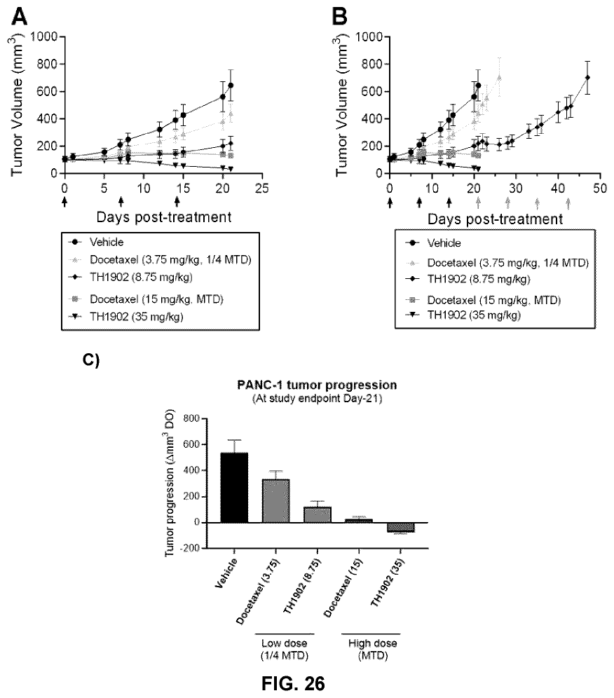

FIG. 26 shows the effect of TH1902 and Docetaxel on Pancreatic Tumor Xenograft Model. Mice bearing pancreatic subcutaneous xenograft tumors were treated with TH1902, docetaxel or with vehicle. PANC-1 tumor volume measurements from all 5 groups were recorded for three weeks and shown in FIG. 26A. The black arrows indicate dates of test article administration. In FIG. 26B, the same data is shown in this panel but includes extended results for the mice administered low dosages of docetaxel and TH1902. All data symbols shown represent means±SEM, n=6 for all groups in panels A and B. The black arrows indicate days of test article administration to all 5 groups; the grey arrows indicate administration to the two low dosage groups of animals only. For FIG. 26C, tumor progression at day-21 was then compared between each treated group. The bars shown represent mean values±SEM. Quadruple asterisks denote p<0.0001.

### Melanoma Cancer Xenograft Model (SK-Mel-28) and Syngeneic Melanoma Model (B16-F10) In Vivo Results

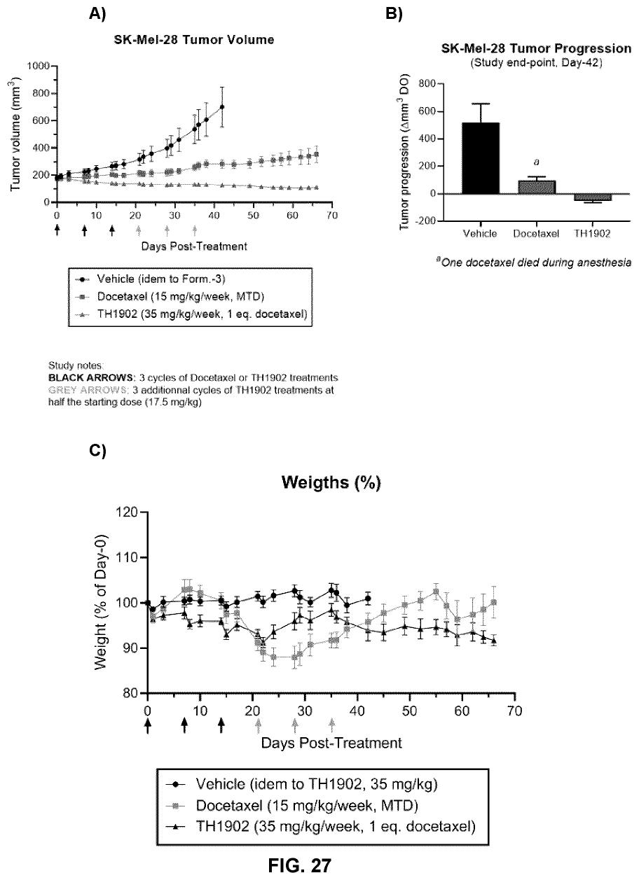

Two of the sortilin-expressing melanoma cancer cell lines (SK-MEL-28 and B16-F10) were used to monitor whether TH1902 impeded the growth of the cells when administered at 35 mg/kg/week, versus unconjugated docetaxel at 15 mg/kg/week. This is the maximum tolerated dose (MTD) for free docetaxel, and the TH1902 formulation contains the equivalent quantity of docetaxel within this peptide drug conjugate. It is apparent that both docetaxel and TH1902 inhibited tumor growth in the SK-MEL-28 xenografts, with TH1902 exhibiting the stronger inhibition (FIG. 27A). This difference was quantified when the control group reached the tumor volume endpoint at day-42. In contrast to docetaxel, a significant regression of tumor volumes was observed between control animals and those treated with TH1902 formulations (FIG. 27B). Body weight of mice treated with vehicle, docetaxel and TH1902 remained within −20% endpoint limit (FIG. 27C).

FIG. 27 shows the effect of TH1902 or docetaxel on the melanoma SK-MEL-28 xenograft tumor model. For FIG. 27A, mice bearing SK-MEL-28 xenografts were repeatedly injected intravenously (arrows indicate injection days) with vehicle or with vehicle containing equivalent of docetaxel (15 mg/kg/week) or TH1902 (35 mg/kg/week). In FIG. 27B, the effects of TH1902 and docetaxel on tumor progression at day-42 are presented by subtracting the initial tumor volumes at day-0 to the tumor volumes measured at day 42. For FIG. 27C body weights of mice treated with vehicle and equivalent doses of docetaxel or TH1902 were monitored during the studies and were within the acceptable range and within the −20% endpoint limit during all the study. All data symbols shown represent means±standard error of the mean (SEM).

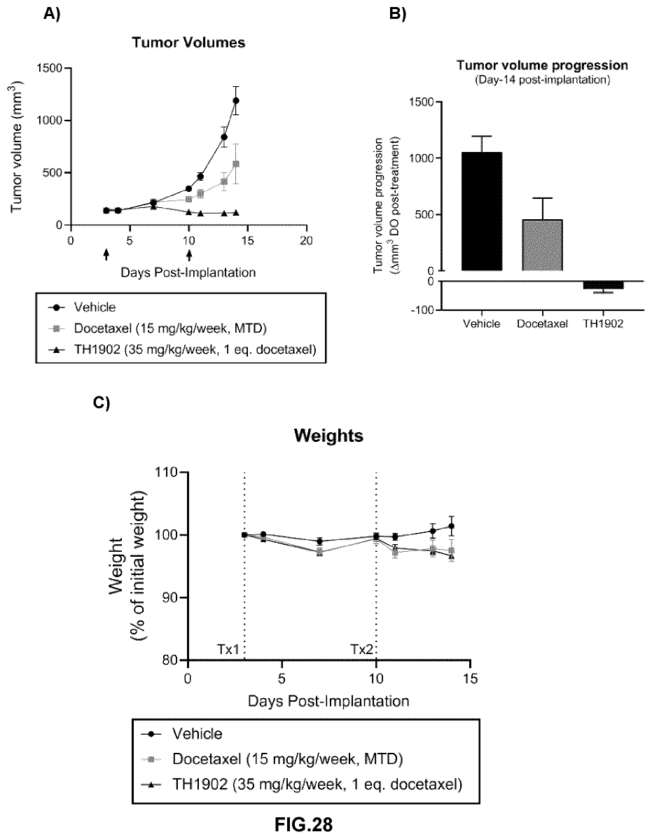

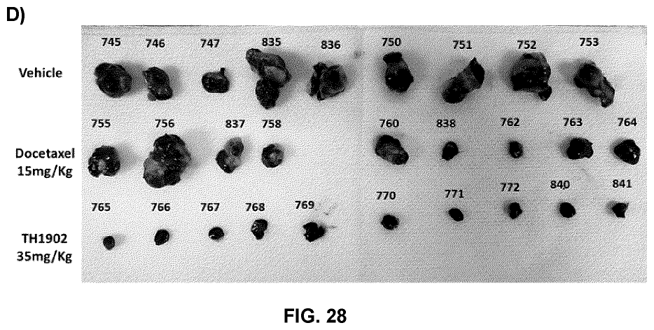

For the syngeneic B16-F10 melanoma tumor model, normal immunocompetent mice were implanted subcutaneously with murine B16-F10 cancer cells. Syngeneic mouse models consist of tumor tissues from the same genetic background as the given immunocompetent mouse strain. A syngeneic mouse model provides an effective approach for studying how cancer therapies perform in the presence of a functional immune system. In a first study (FIG. 28), mice implanted with B16-F10 cancer cells were treated weekly with vehicle and either docetaxel or TH1902 at equivalent doses. Results in FIG. 28A show that this model is very aggressive and that tumors are growing very rapidly. In this very aggressive melanoma syngeneic tumor model, a better efficacy for TH1902 formulations was clearly observed when compared to unconjugated docetaxel. At study endpoint on day-14 (FIG. 28B), tumor regressions were measured in mice treated with TH1902, in contrast to docetaxel without having an impact on mouse body weights (FIG. 28C). In addition, tumors were collected on day-14, aligned and photographed (FIG. 28D). Pictures clearly demonstrate huge differences in the size of tumors treated with TH1902 formulations when compared to those treated with the vehicle or docetaxel. All tumors treated with TH1902 formulations were much smaller than tumors from the two other groups (vehicle and docetaxel).

FIG. 28 shows the effect of TH1902 or docetaxel on the syngeneic B16-F10 xenograft tumor model. For FIG. 28A, immunocompetent mice bearing B16-F10 xenografts were repeatedly injected intravenously (arrows indicate injection days) with vehicle or with vehicle containing equivalent of docetaxel (15 mg/kg/week) or TH1902 (35 mg/kg/week). In FIG. 28B, the effects of TH1902 and docetaxel on tumor progression at day-14 are presented by subtracting the initial tumor volumes at day-0 to the tumor volumes measured at day-14 when tumors of the vehicle group reached the endpoint limits. For FIG. 24C, body weights of mice treated with vehicle and equivalent doses of docetaxel or TH1902 were monitored during the studies and were within the acceptable range and within the −20% endpoint limit during all the study. For FIG. 28D, tumors were collected at day-14, aligned accordingly to their treatments and photographed. All data symbols shown represent means±standard error of the mean (SEM).

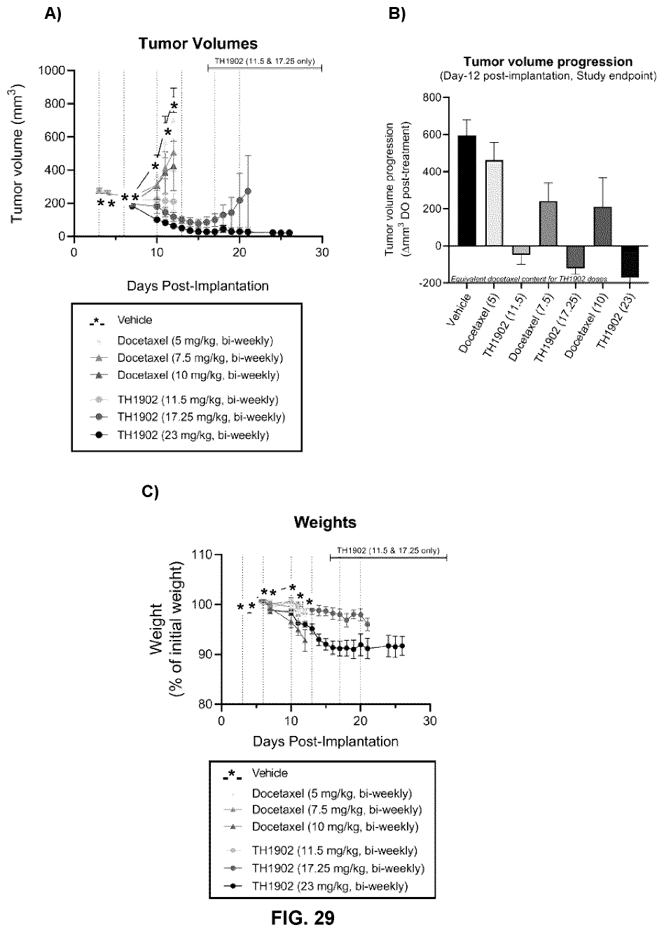

A dose-response was next performed in a second study with the syngeneic B16-F10 melanoma tumor model (FIG. 29). Three different equivalent doses of docetaxel and TH1902 were administered by IV bolus injection to immunocompetent mice bearing B16-F10 tumors. Mice were then treated at 5, 7.5 and 10 mg/kg/bi-weekly of docetaxel and equivalent TH1902 (11.5, 17.25 and 23 mg/kg/bi-weekly). Results clearly demonstrate that TH1902 induced a much stronger tumor growth inhibition at all doses when compared to unconjugated docetaxel. At the highest dose of TH1902 (23 mg/kg/bi-weekly) clear and long-lasting regressions were observed. The equivalent dose of docetaxel (10 mg/kg/bi-weekly) has only a minor impact on tumor growth.

FIG. 29 shows TH1902 or docetaxel dose-response study on the syngeneic B16-F10 xenograft tumor model. For FIG. 29A, immunocompetent mice bearing B16-F10 tumor xenografts were repeatedly injected intravenously (dotted lines indicate injection days) with vehicle or with equivalent increasing doses of TH1902 and docetaxel. Mice were treated twice a week with docetaxel at 5, 7.5 and 10 mg/kg and equivalent TH1902 doses (11.5, 17.25 and 23 mg/kg). In FIG. 29B, effects of TH1902 and docetaxel on tumor progression at day-12 are presented by subtracting the initial tumor volumes at day-0 to the tumor volumes measured at day-12 when tumors of the vehicle group reached the endpoint limits. In contrast to docetaxel, tumor regressions were clearly observed for TH1902 formulations. For FIG. 29C, body weights of mice treated with vehicle and equivalent doses of docetaxel or TH1902 were monitored during the studies and were within the acceptable range and within the −20% endpoint limit during all the study.

The embodiments of the present disclosure are presented in such a manner in the present disclosure so as to demonstrate that every combination of embodiments, when applicable, can be made. These embodiments have thus been presented in the description in a manner equivalent to making dependent claims for all the embodiments that depend upon any of the preceding claims (covering the previously presented embodiments), thereby demonstrating that they can be combined together in all possible manners. For example, all the possible combinations, when applicable, between the embodiments and the various aspects presented in paragraphs herein are hereby covered by the present disclosure.

### REFERENCES

1. World Cancer Report, 2014.
2. Urruticoechea A, Alemany R, Balart J, Villanueva A, Vinals F, Capella G. Recent advances in cancer therapy: an overview. Curr Pharm Des 2010; 16:3-10.
3. Fisher R, Pusztai L, Swanton C. Cancer heterogeneity: implications for targeted therapeutics. Brit J Cancer 2013; 108; 479-485.
4. Zhao P, Astruc D. Docetaxel nanotechnology in anticancer therapy. Chem Med Chem 2012; 7:952-72.
5. Ophir E, Bobisse S, Coukos G, Harari A, Kandalaft L E. Personalized approaches to active immunotherapy in cancer. Biochim Biophys Acta 2016; 1865:72-82.
6. Sapiezynski J, Taratula O, Rodriguez-Rodriguez L, Minko T. Precision targeted therapy of ovarian cancer. J Cont Rel 2016; 243:250-268.
7. Wilson C M, Naves T, Saada S, Pinet S, Vincent F, Lalloué F, Jauberteau M O. The implications of sortilin/vps10p domain receptors in neurological and human diseases. CNS Neurol Disord Drug Targets 2014; 13:1354-65.
8. Vincent J P, Mazella J, Kitabgi P. Neurotensin and neurotensin receptors. Trends Pharmacol Sci 1999; 20:302-309.
9. Carlo A S, Nykjaer A, Willnow T E. Sorting receptor sortilin-a culprit in cardiovascular and neurological diseases. J Mol Med 2014; 92:905-11.
10. Schmidt V, Willnow T E. Protein sorting gone wrong-VPS10P domain receptors in cardiovascular and metabolic diseases. Atheroscler 2016; 245:194-9.
11. Wilson C M, Naves T, Al Akhrass H, Vincent F, Melloni B, Bonnaud F, Lalloué F, Jauberteau M O. A new role under sortilin's belt in cancer. Com Integr Biol 2016; 9:e1130192.
12. AI-Shawi R, Hafner A, Chun S, Raza S, Crutcher K, Thrasivoulou C, Simons P, Cowen T. ProNGF, sortilin, and age-related neurodegeneration. Ann N Y Acad Sci 2007; 1119:208-15.
13. Lewin G R, Nykjaer A. Pro-neurotrophins, sortilin, and nociception. Eur J Neurosci 2014; 39:363-74.
14. Mazella J, Vincent J P. Internalization and recycling properties of neurotensin receptors. Peptides 2006; 27:2488-92.
15. Vaegter C B, Jansen P, Fjorback A W, Glerup S, Skeldal S, Kjolby M, Richner M, Erdmann B, Nyengaard J R, Tessarollo L, et al. Sortilin associates with Trk receptors to enhance anterograde transport and neurotrophin signaling. Nat Neurosci 2011; 14:54-61.
16. Akil H, Perraud A, Melin C, Jauberteau M O, Mathonnet M. Fine-tuning roles of endogenous brain-derived neurotrophic factor, TrkB and sortilin in colorectal cancer cell survival. PloS One 2011; 6:e25097.
17. Dal Farra C, Sarret P, Navarro V, Botto J M, Mazella J, Vincent J P. Involvement of the neurotensin receptor subtype NTR3 in the growth effect of neurotensin on cancer cell lines. Int J Cancer 2001; 92:503-9.
18. Truzzi F, Marconi A, Lotti R, Dallaglio K, French L E, Hempstead B L, Pincelli C. Neurotrophins and their receptors stimulate melanoma cell proliferation and migration. J Invest Dermatol 2008; 128:2031-40.
19. Giorgi R R, Chile T, Bello A R, Reyes R, Fortes M A, Machado M C, Cescato V A, Musolino N R, Bronstein M D, Giannella-Neto D, et al. Expression of neurotensin and its receptors in pituitary adenomas. J Neuroendocrinol 2008; 20:1052-7.
20. Xiong J, Zhou L, Yang M, Lim Y, Zhu Y H, Fu D L, Li Z W, Zhong J H, Xiao Z C, Zhou X F. ProBDNF and its receptors are upregulated in glioma and inhibit the growth of glioma cells in vitro. Neuro Oncol 2013; 15:990-1007.
21. Hemmati S, Zarnani A H, Mahmoudi A R, Sadeghi M R, Soltanghoraee H, Akhondi M M, Tarahomi M, Jeddi-Tehrani M, Rabbani H. Ectopic Expression of Sortilin 1 (NTR-3) in Patients with Ovarian Carcinoma. Avicenna J Med Biotechnol 2009; 1:125-31.
22. Ghaemimanesh F, Ahmadian G, Talebi S, Zarnani A H, Behmanesh M, Hemmati S, Hadavi R, Jeddi-Tehrani M, Farzi M, Akhondi M M, Rabbani H. The effect of sortilin silencing on ovarian carcinoma cells. Avicenna J Med Biotechnol 2014; 6:169-77.
23. Hosseini A, Ghorbani A. Cancer therapy with phytochemicals: evidence from clinical studies. Avicenna J Phytomed. 2015; 5:84-97.
24. Cao Z, Bao M, Miele L, Sarkar F H, Wang Z, Zhou Q. Tumour vasculogenic mimicry is associated with poor prognosis of human cancer patients: a systemic review and meta-analysis. Eur J Cancer. 2013 49:3914-23.
25. Kirschmann D A, Seftor E A, Hardy K M, Seftor R E, Hendrix M J. Molecular pathways: vasculogenic mimicry in tumor cells: diagnostic and therapeutic implications. Clin Cancer Res. 2012; 18:2726-32.
26. Clarijs R, Otte-Höller I, Ruiter D J, de Waal R M. Presence of a fluid-conducting meshwork in xenografted cutaneous and primary human uveal melanoma. Invest Ophthalmol Vis Sci. 2002; 43:912-8.
27. Kobayashi H, Shirakawa K, Kawamoto S, Saga T, Sato N, Hiraga A, Watanabe I, Heike Y, Togashi K, Konishi J, Brechbiel M W, Wakasugi H. Rapid accumulation and internalization of radiolabeled herceptin in an inflammatory breast cancer xenograft with vasculogenic mimicry predicted by the contrast-enhanced dynamic MRI with the macromolecular contrast agent G6-(1 B4M-Gd)(256). Cancer Res. 2002; 62:860-6.
28. Maniotis AJ1, Chen X, Garcia C, DeChristopher P J, Wu D, Pe'er J, Folberg R. Control of melanoma morphogenesis, endothelial survival, and perfusion by extracellular matrix. Lab Invest. 2002; 82:1031-43.
29. Qiao L, Liang N, Zhang J, Xie J, Liu F, Xu D, Yu X, Tian Y. Advanced research on vasculogenic mimicry in cancer. J Cell Mol Med. 2015; 19:315-26.
30. Liu T J, Sun B C, Zhao X L, Zhao X M, Sun T, Gu Q, Yao Z, Dong X Y, Zhao N, Liu N. CD133+ cells with cancer stem cell characteristics associates with vasculogenic mimicry in triple-negative breast cancer. Oncogene. 2013; 32:544-53.
31. Racordon D, Valdivia A, Mingo G, Erices R, Aravena R, Santoro F, Bravo M L, Ramirez C, Gonzalez P, Sandoval A, Gonzalez A, Retamal C, Kogan M J, Kato S, Cuello M A, Osorio G, Nualart F, Alvares P, Gago-Arias A, Fabri D, Espinoza I, Sanchez B, Corvalan A H, Pinto M P, Owen G I. Structural and functional identification of vasculogenic mimicry in vitro. Sci Rep. 2017; 7:6985.
32. Chiablaem K, Lirdprapamongkol K, Keeratichamroen S, Surarit R, Svasti J. Curcumin suppresses vasculogenic mimicry capacity of hepatocellular carcinoma cells through STAT3 and PI3K/AKT inhibition. Anticancer Res. 2014; 34:1857-64.
33. McCafferty J, Griffiths A D, Winter G, Chiswell D J. Phage antibodies: filamentous phage displaying antibody variable domains. Nature 1990; 348: 552-554.
34. Carter P, Merchant A M. Engineering antibodies for imaging and therapy. Current Opinion in Biotechnology 1997; 8:449-454.
35. Riechmann L, Clark M, Waldmann H, Winter G. Reshaping human antibodies for therapy. Nature 1988; 332:323-327.
36. Foote J, Winter G. Antibody framework residues affecting the conformation of the hypervariable loops. Journal of Molecular Biology 1992; 224:487-499.
37. Yano S, Hsu R K, Landolfi N F, Vasquez M, Cole M, Tso J T, Bringman T, Laird W, Hudson D. A humanized antibody specific for the platelet integrin gpIIb/IIIa. The Journal of Immunology 1994; 152:2968-2976.
38. Pedersen J T, Henry A H, Searle S J, Guild B C, Roguska M, Rees A R. Comparison of surface accessible residues in human and murine immunoglobulin Fv domains: implication for humanization of murine antibodies. Journal of molecular biology 1994; 235:959-973.
39. Köhler G, Milstein C. Continuous cultures of fused cells secreting antibody of predefined specificity. Nature 1975, 256: 495-497.
40. Kozbor D, Roder J C. The production of monoclonal antibodies from human lymphocytes. Immunology Today 1983, 4: 72-79.
41. Cole S P C, Kozbor D, Roder J C. The EBV-hybridoma technique and its application to human lung cancer. In: Reisfeld R A, Sell S, eds. Monoclonal Antibodies and Cancer Therapy. New York: Alan R. Liss Inc. (UCLA Symposia on Molecular and Cellular Biology) 1985, 27:77-96.
42. Ge H, Luo H. Overview of advances in vasculogenic mimicry—a potential target for tumor therapy. Cancer Manag Res. 2018; 10:2429-2437.
43. Zhou Q, Zhifei C, Meimei B, Miele L, Sarkar F H, Wang Z. Tumour vasculogenic mimicry is associated with poor prognosis of human cancer patients: A systemic review and meta-analysis. Eur J Cancer 2013; 49: 3914-3923.
44. Yang, J. P. et al. Tumor vasculogenic mimicry predicts poor prognosis in cancer patients: a meta-analysis. Angiogenesis 2016; 19: 191-200.
45. Sun B, Zhang D, Zhao N and Zhao X. Epithelial-to-endothelial transition and cancer stem cells: two cornerstones of vasculogenic mimicry in malignant tumors. Oncotarget 2017; 8: 30502-30510.
46. Liang J, Yang B, Cao Q, Wu X. Association of Vasculogenic Mimicry Formation and CD133 Expression with Poor Prognosis in Ovarian Cancer. Gynecol Obstet Invest. 2016; 81:529-536.
47. Altschul et al., 1997, Nucleic Acids Res. 25:3389-3402.
48. Navarro et al., 2002.
49. Altschul et al., 1990, J. Mol. Biol. 215:403.
50. Bissery et al. 1995.
51. Clarke et al. 1999.
52. Karlin and Altschul, 1990, Proc. Natl. Acad. Sci. U.S.A. 87:2264-2268.
53. Karlin and Altschul, 1993, Proc. Natl. Acad. Sci. U.S.A. 90:5873-5877.

1-138. (canceled)

139. A composition comprising
(i) a conjugate compound or a pharmaceutically acceptable salt, solvate or prodrug thereof, said conjugate compound having the formula of A-(B)ₙ,
wherein
A is a peptide compound comprising a sequence having at least 60% sequence identity with the sequence GVRAKAGVRN(Nle)FKSESY (SEQ ID NO: 10), optionally protected by a protecting group;
B is at least one therapeutic agent; and
n is 1, 2, 3 or 4; and
(ii) a polysorbate.

140. The composition of claim 139, wherein the peptide compound comprises a sequence having at least 90% sequence identity with the sequence

| (SEQ ID NO: 10) | |
|---|---|
| | GVRAKAGVRN(Nle)FKSESY |

141. The composition of claim 139, wherein the peptide compound comprises or consists of the sequence GVRAKAGVRN(Nle)FKSESY (SEQ ID NO: 10).

142. The composition of claim 139, wherein the peptide compound comprises at least one protecting group that is acetyl or succinyl.

143. The composition of claim 139, wherein the peptide compound is represented by Formula (XXXIX):

| | (XXXIX) |
|---|---|
| (SEQ ID NO: 15) | |
| | Acetyl-GVRAKAGVRN(Nle)FKSESY |

144. The composition of claim 139, wherein B is connected to A via a linker.

145. The composition of claim 139, wherein the at least one therapeutic agent is an anticancer agent.

146. The composition of claim 145, wherein the anticancer agent is docetaxel, doxorubicin, cabazitaxel, maytansinoids, auristatins, calicheamicins, amatoxins, amanitin, or aldoxorubicin.

147. The composition of claim 146, wherein the conjugate compound is represented by formula (XIX) or (XXIII):
Formula (XIX)
GVRAK(docetaxel)AGVRN(Nle)FK(docetaxel)SESY;
Formula (XXIII)
Acetyl-GVRAK(docetaxel)AGVRN(Nle)FK(docetaxel)
SESY

148. The composition of claim 139, wherein the conjugate compound is present in an amount from about 0.1% to about 5% by w/w % based on the total weight of the composition.

149. The composition of claim 139, wherein the polysorbate is polysorbate 80.

150. The composition of claim 139, wherein the polysorbate is present in an amount from about 5% to about 15% by weight per total volume of the composition.

151. The composition of claim 139, further comprising dextrose.

152. The composition of claim 151, wherein the dextrose is present in an amount from about 2% to about 8% by weight per total volume of the composition.

153. The composition of claim 151, wherein the dextrose is present in an amount of about 5% by weight per total volume of the composition.

154. The composition of claim 139, further comprising a buffering agent.

155. The composition of claim 154, wherein the buffering agent is formic acid.

156. The composition of claim 139, wherein the composition is an aqueous solution having a pH of about 3.5 to about 4.5.

157. The composition of claim 139, wherein said conjugate compound is in the form of a pharmaceutically acceptable acid addition salt.

158. The composition of claim 157, wherein said pharmaceutically acceptable acid addition salt is acetate or formate.

159. A method of treating a Sortilin-expressing cancer comprising administering to a subject in need thereof a therapeutically effective amount of the composition as defined in claim 139.
